(19) United States
(12) Patent Application Publication
(10) Pub. No.: US 2015/0258170 A1
(43) Pub. Date: Sep. 17, 2015

(54) Diagnosis and Treatment of SMA and SMN Deficiency

(71) Applicant: THE TRUSTEES OF COLUMBIA UNIVERSITY IN THE CITY OF NEW YORK, New York,, NY (US)

(72) Inventors: Brian D. McCabe, New York, NY (US); Livio Pellizzoni, New York, NY (US)

(73) Assignee: The Trustees of Columbia University in the City of New York, New York, NY (US)

(21) Appl. No.: 14/434,501

(22) PCT Filed: Oct. 10, 2013
(86) PCT No.: PCT/US13/64415

§ 371 Date: Apr. 9, 2015

Related U.S. Application Data
(60) Provisional application No. 61/712,220, filed on Oct. 10, 2012.

(51) Int. Cl. A61K 38/17; C12Q 1/68; C12N 15/86

(52) U.S. Cl. CPC A61K 38/177; C12N 15/86; C12Q 1/6883; A61K 48/00

(57) ABSTRACT

The present invention provides for methods for diagnosing and treating a motor neuron disease. More specifically, the present invention offers new methods for diagnosing and treating SMA or SMN deficiencies and monitoring treatment. It is possible to identify a subject having a symptom of the disease, and then administer to the subject a therapeutically effective amount of one or more proteins or a gene delivery vehicle or pharmaceutical composition comprising one or more genes selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1.

### CROSS-REFERENCE TO RELATED APPLICATIONS

This application claims benefit of U.S. Provisional Application Ser. No. 61/712,220, entitled “Diagnosis and Treatment of SMA and SMN Deficiency,” filed Oct. 10, 2012, the entire contents of which are hereby incorporated by reference as if fully set forth herein, under 35 U.S.C. §119(e).

### STATEMENT OF GOVERNMENTAL INTEREST

This invention was made with Government support under Contract No. W81XWH-08-1-0009 awarded by the Department of Defense and Contracts No. R01NS069601 and 1R21NS077038-01A1 awarded by the National Institutes of Health. The Government has certain rights in the invention.

### BACKGROUND OF THE INVENTION

The motor neuron diseases (MNDs) are a group of progressive neurological disorders that destroy motor neurons, the cells that control essential voluntary muscle activity such as speaking, walking, breathing, and swallowing. Normally, messages from motor nerve cells in the brain (called upper motor neurons) are transmitted to motor nerve cells in the brain stem and spinal cord (called lower motor neurons) and from the lower motor neurons messages are transmitted to particular muscles. Upper motor neurons direct the lower motor neurons to produce movements such as walking or chewing. Lower motor neurons control movement in the arms, legs, chest, face, throat, and tongue. Spinal motor neurons are also called anterior horn cells. Upper motor neurons are also called corticospinal neurons.

MNDs are classified as either inherited or sporadic, and according to whether degeneration affects upper motor neurons, lower motor neurons, or both. In adults, the most common MND is amyotrophic lateral sclerosis (ALS), which affects both upper and lower motor neurons.

Spinal Muscular Atrophy (“SMA”) is diagnosed when clinical features become apparent and is confirmed with genetic tests. SMA is characterized by degeneration of motor neurons and atrophy of skeletal muscle. The nearly identical SMN2 gene is unable to compensate for the loss of SMN1 as it produces low levels of functional spinal motor neuron (“SMN”) protein. Consistent with human pathology, in both invertebrate and vertebrate animal models, low levels of SMN are sufficient for normal function of most cell types but not of the motor system (Burghes and Beattie, 2009). However, the mechanisms that link ubiquitous SMN deficiency to selective neuronal dysfunction remain unclear.

Currently, there are no therapies that directly target the pathogenesis of SMA. Treatment includes palliative care by alleviating symptoms of SMA. The present invention offers new methods for diagnosing and treating SMA or SMN deficiencies and monitoring treatment.

Other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### SUMMARY OF THE INVENTION

The present invention is directed to methods for diagnosis and treatment of a MND (e.g., SMA, ALS, Progressive bulbar pals, Pseudobulbar palsy, Primary lateral sclerosis (PLS), and Progressive muscular atrophy). The subject (e.g., mammalian, human) is identified as having a symptom of the disease and then administered a therapeutically effective amount of one or more proteins or a gene delivery vehicle (e.g., AAV) that includes Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1. If a protein is used, the therapeutically effective amount of the protein is an amount that increases transmission from a neuromuscular junction or increases muscle mass in the subject. The protein may be Stasimon, Rashomon or a combination thereof or a biologically active fragment or variant thereof. Genes encoding Stasimon, Rashomon or both, or biologically fragments or variants thereof are also contemplated. The gene delivery vehicle is a viral vector comprising one or more genes as cDNAs. In certain embodiments, the gene delivery vehicle that is AAV may comprise a single stranded or a self-complementary genome comprising one or more genes as cDNAs. The gene delivery vehicle may be administered systemically and to the brain, spinal cord or to a motor circuit neuron.

In certain embodiments, methods are described for diagnosing a MND by identifying a subject with a symptom of a MND. Once the subject with a symptom of a MND is identified, a biological sample (e.g., fibroblasts, blood, serum, muscle and cerebrospinal fluid) is obtained from the subject. A control biological sample from a normal subject known not to have a motor neuron disease is also obtained. It is then determined in the subject sample and the control samples a level of a mRNA comprising an unspliced, abnormally spliced, or aberrantly spliced U12 intron which is transcribed from a U12 intron-containing gene selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or a level of correctly spliced mRNA encoding the one or more proteins. If the level of protein (determined by e.g., western blot, or immunohistochemical analyses using antibodies raised against the protein) or correctly spliced mRNA is significantly lower in the subject sample than in the control sample, then the subject is determined to have the MND. Ultimately, the subject with the MND can be treated. It is also contemplated that upon diagnosis and treatment, the treatment may be continued for a period of time for the MND.

In other embodiments, methods are disclosed for monitoring a response of a subject to treatment for a MND such as those described herein. This is accomplished by obtaining a pretreatment biological sample and a post treatment biological sample from the subject. Then it is determined whether in the pretreatment and post treatment samples there is a level of an mRNA (e.g., determined by PCR) comprising an unspliced, abnormally spliced, or aberrantly spliced U12 intron which mRNA is transcribed from a U12 intron-containing gene selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or a level of correctly spliced mRNA encoding the one or more proteins. If the level of the protein or mRNA is significantly higher in the post treatment sample than in the pretreatment sample, then it is possible to determine that the subject is responding to treatment for the MND, and continuing the treatment for the MNF. The treatment may be a SMN restoration therapy comprising administering a gene encoding SMN to the subject.

In certain embodiments, the treatment of the subject may include administering to the subject a therapeutically effective amount of the protein determined to be present in the subject biological sample at a level that is significantly lower than in the control level of the protein.

In other embodiments, an gene vehicle such as AAV comprises a cDNA of a gene encoding one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1.

In yet other embodiments, pharmaceutical formulations comprise one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1. The pharmaceutical formulation may be used for uptake by the brain, spinal cord, or motor circuit neurons.

In certain embodiments, microarrays comprise two or more oligonucleotides bound to a support that are complementary to and selectively hybridize to one or more genes selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or to one or more correctly spliced mRNAs encoded by the respective genes.

In certain preferred embodiments, the invention relates to pharmaceutical compositions comprising a pharmaceutically acceptable vehicle and a therapeutically effective amount of one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, Nucleolar protein 1, and any combination thereof, and any biologically active fragment or variant thereof.; and to methods for treating a motor neuron disease, comprising identifying a subject having a symptom of the disease, and administering to the subject this pharmaceutical composition. Preferably the compositions are formulated for uptake by the brain, spinal cord or motor circuit neurons. Certain methods involve administration of proteins such as Stasimon, Rashomon or a combination thereof, or a biologically active fragment or variant thereof. The therapeutically effective amount of the protein preferably is an amount that increases muscle mass in the subject or that increases transmission from a neuromuscular junction.

Embodiments of the invention relate to methods for treating a motor neuron disease, comprising identifying a subject having a symptom of the disease, and administering to the subject a gene delivery vehicle comprising one or more genes selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, Nucleolar protein 1 and a biologically active fragment or variant thereof. The subject can be mammalian and preferabally is human.

The motor neuron disease preferably is a member selected from the group comprising Amyotrophic lateral sclerosis (ALS), Progressive bulbar pals, Pseudobulbar palsy, Primary lateral sclerosis (PLS), Progressive muscular atrophy and spinal muscular atrophy (SMA). Treatment can be SMN restoration therapy comprising administering a gene encoding SMN to the subject. The gene delivery vehicle preferably is a viral vector comprising the one or more genes as cDNAs and can be an AAV comprising either a single stranded or a self-complementary genome comprising the one or more genes as cDNAs. Delivery can be by administering systemically, to the brain, to the spinal cord or to a motor circuit neuron.

In addition, other preferred embodiments of the invention relate to a method for diagnosing a motor neuron disease comprising identifying a subject with a symptom of a motor neuron disease, obtaining a biological sample from the subject and a control biological sample from a normal subject known not to have a motor neuron disease, determining in the subject sample and the control samples a level of an mRNA comprising an unspliced, abnormally spliced, or aberrantly spliced U12 intron which is transcribed from a U12 intron-containing gene selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or a level of correctly spliced mRNA encoding the one or more proteins; if the level of protein or correctly spliced mRNA is significantly lower in the subject sample than in the control sample, then determining that the subject has the motor neuron disease, and treating the subject the motor neuron disease.

In addition, an embodiment of the invention relates to a 3. A method for diagnosing a motor neuron disease comprising identifying a subject with a symptom of a motor neuron disease, obtaining a biological sample from the subject and a control biological sample from a normal subject known not to have a motor neuron disease, determining in the subject sample and the control samples a level of one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or a level of correctly spliced mRNA encoding the one or more proteins; if the level of protein or correctly spliced mRNA is significantly lower in the subject sample than in the control sample, then determining that the subject has the motor neuron disease, and treating the subject the motor neuron disease.

A further embodiment relates to a method for monitoring a response of a subject to treatment for a motor neuron disease, comprising obtaining a pretreatment biological sample and a post treatment biological sample from the subject, determining in the pretreatment and post treatment samples a level of one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or a level of correctly spliced mRNA encoding the one or more proteins; and if the level of the protein or mRNA s significantly higher in the post treatment sample than in the pretreatment sample, then determining that the subject is responding to treatment for the motor neuron disease, and continuing the treatment for the motor neuron disease.

A further embodiment of the invention relates to a method for monitoring a response of a subject to treatment for a motor neuron disease, comprising obtaining a pretreatment biological sample and a post treatment biological sample from the subject, determining in the pretreatment and post treatment samples a level of an mRNA comprising an unspliced, abnormally spliced or aberrantly spliced U12 intron which mRNA is transcribed from a U12 intron-containing gene selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or a level of correctly spliced mRNA encoding the one or more proteins; and if the level of the protein or mRNA s significantly higher in the post treatment sample than in the pretreatment sample, then determining that the subject is responding to treatment for the motor neuron disease, and continuing the treatment for the motor neuron disease.

Biological samples useful in these method above can be a member selected from the group comprising fibroblasts, blood, serum, muscle and cerebrospinal fluid.

The methods of preferred embodiments may further include treating the subject by administering to the subject a therapeutically effective amount of the protein determined in other steps to be present in the subject biological sample at a level that is significantly lower than in the control level of the protein.

Suitable biological samples for use with any of the inventive methods is a member selected from the group comprising fibroblasts, blood, serum, muscle and cerebrospinal fluid of claim 23 wherein the biological sample is a member selected from the group comprising fibroblasts, blood, serum, muscle and cerebrospinal fluid.

Further preferred embodiments of the invention include an AAV comprising a cDNA of a gene encoding one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1; and a microarray comprising two or more oligonucleotides bound to a support that are complementary to and selectively hybridize to one or more genes selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or to one or more correctly spliced mRNAs encoded by the respective genes.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1. (A) Western blot analysis of NIH3T3-Smn_{RNAi} and NIH3T3-SMN/Smn_{RNAi} cells cultured without (−) or with (+) Dox for 5 days. (B) RT-qPCR analysis of snRNAs immunoprecipitated with anti-SmB antibodies from NIH3T3-Smn_{RNAi} and NIH3T3-SMN/Smn_{RNAi} cells cultured as in (A). RNA levels in Dox-treated cells were expressed relative to untreated cells (dotted line). (C) RT-PCR analysis of U12 intron-containing genes in NIH3T3-Smn_{RNAi} and NIH3T3-SMN/Smn_{RNAi} cells cultured as in (A). Genes and exons monitored by PCR are indicated on the left. Schematics of spliced and intron-containing mRNAs are shown on the right. Callout lines highlight U12 introns. The asterisk indicates a Tmem4lb mRNA spliced using donor and acceptor sites located at −30 and +25, respectively, relative to the U12 intron splice sites. (D) RT-qPCR analysis of U12 intron retention for a subset of genes in (C). (E) RT-qPCR analysis of mRNA expression for a subset of genes in (C). (F) RT-qPCR analysis of aberrantly spliced Tmem41b and exon-skipped Clcn7 mRNAs. For all RT-qPCR experiments, NIH3T3 cells were cultured as in (A) and RNA levels in Dox-treated cells were expressed relative to untreated cells (dotted line). See also FIG. 8 and Table 1.

FIG. 2. (A) Western blot analysis of NIH3T3-Smn_{RNAi} cells cultured with Dox for the indicated number of days. A two-fold serial dilution of the extract from uninduced cells is shown on the left. (B) RT-qPCR analysis of U12 intron retention in Clcn7, Parp1, Tspan31 and Tmem41b mRNAs as well as accumulation of abnormally spliced Tmem41b and Clcn7 mRNAs in NIH3T3-Smn_{RNAi} cells cultured as in (A). RNA levels in Dox-treated cells were expressed relative to untreated cells (dotted line). (C) SMN deficiency decreases proliferation of NIH3T3 cells. Equal numbers of NIH3T3-Smn_{RNAi} cells were cultured with or without Dox for the indicated number of days and cell number determined at each time point. See also FIG. 9.

FIG. 3. (A) Schematic representation of Drosophila smn^{73Ao} and U6atac^{K01105} mutants. (B) Western blot analysis of control, smn^{73Ao} and U6atac^{K01105} larvae. A two-fold serial dilution of control extract is shown on the left. The asterisk indicates a non-specific protein. (C) Northern blot analysis of snRNA expression in control, smn^{73Ao} and U6atac^{K01105} larvae. (D) snRNA levels in smn^{73Ao} and U6atac^{K01105} relative to control (dotted line) larvae following normalization to 5.8S rRNA. (E) RT-PCR analysis of U12 intron-containing genes whose expression is affected in both smn^{73Ao} and U6atac^{K01105} compared to control larvae. Genes and exons monitored by PCR are indicated on the left. Schematics of spliced and intron-containing mRNAs are shown on the right. Call out lines highlight U12 introns. The −RT lanes correspond to RT-PCR reactions lacking reverse transcriptase. (F) RT-qPCR analysis of U12 intron-containing genes with decreased mRNA expression in both smn^{73Ao} and U6atac^{K01105} compared to control larvae. (G) RT-qPCR analysis of genes with increased U12 intron retention in U6atac^{K01105} compared to control larvae. (H) RT-qPCR analysis of genes with increased U12 intron retention in smn^{73Ao} compared to control larvae. See also FIG. 10 and Table 2.

FIG. 4. (A) Evoked Excitatory Post-Synaptic Potentials (eEPSPs) in smn^{73Ao} and smn^{X7} mutant Drosophila larvae and following mRNA knockdown of the indicated genes by pan-neural expression of UAS-RNAi constructs with C155-Gal4 normalized to control. (B₁-B₃) Schematic representation of the stas^{EY04008} mutant showing the site of P-element insertion within the 5′ UTR region of the stasimon (CG8408) gene (B₁). RT-qPCR analysis of Stasimon mRNA levels in control and stas^{EY04008} larvae (B₂). Normalized eEPSP amplitude in control and stas^{EY04008} larvae with or without expression of UAS-Stasimon with the pan-neuronal nsyb-Gal4 driver relative to control (B₃). (C) Representative eEPSP traces from larvae with Stasimon RNAi in all neurons (C155-Ga14; PAN), motor neurons (OK371-Gal4; MN) or cholinergic neurons (Cha-Gal4; CHOL) normalized to control. (D) Quantification of eEPSP amplitudes in larvae with Stasimon RNAi in specific neuronal types normalized to control. See also FIG. 11.

FIG. 5. (A-B) Representative eEPSP traces recorded from muscle 6 of segment A3 in control and smn^{X7} larvae. (C) Normalized eEPSP amplitude of smn^{X7} mutants alone or with transgenic UAS-Stasimon expression in all neurons (nsyb-Gal4; PAN), motor neurons (OK371-Gal4; MN) or cholinergic neurons (Cha-Gal4; CHOL) relative to controls. (D-E) Representative images of muscles from segment A3 of control and smn^{X7} larvae labeled with TRITC-phalloidin. (F) Normalized muscle surface area of smn^{X7} mutants alone or expressing Stasimon with the same drivers described in (C) relative to controls. (G-H) Representative recordings of motor rhythms from control and smn^{X7} larvae. (I) Normalized inter-spike intervals of smn^{X7} mutants alone or expressing Stasimon with the same drivers described in (C) relative to controls. (J-K) Representative images of 10 superimposed locomotion path traces from control and smn^{X7} larvae. (L) Normalized path length of smn^{X7} mutants alone or expressing Stasimon with the same drivers described in (C) relative to controls. See also FIG. 12.

FIG. 6. (A) Representative lateral views of motor axons in Tg(mnx1:GFP) zebrafish embryos expressing GFP in motor neurons and injected with Control MO as well as stas MO, smn MO and tdp43 MO either with or without co-injected human STAS RNA. (B) Quantification of the effects of Stasimon deficiency on motor axon development in zebrafish. Motor axons were scored in Tg(mnx1:GFP) embryos injected with Control MO, stas MO or stas MO+STAS RNA. Embryos were classified as severe, moderate, mild or no defects based on the severity of motor axons defects and the percentage for each group is shown. (C) Quantification of Stasimon effects on SMN-dependent motor axons defects in zebrafish. Motor axons were scored in Tg(mnx1:GFP) embryos injected with Control MO, smn MO or smn MO+STAS RNA and embryos were classified as in (B). (D) Quantification of Stasimon effects on TDP43-dependent motor axons defects in zebrafish. Motor axons were scored in Tg(mnx1:GFP) embryos injected with Control MO, tdp43 MO or tdp43 MO+STAS RNA and embryos were classified as in (B). See also FIG. 13.

FIG. 7. (A) RT-qPCR analysis of aberrantly spliced Stasimon mRNA in the spinal cord and L1 DRG from control and SMA mice at the indicated post-natal days. (B). RT-qPCR analysis of Stasimon U12 intron retention in the spinal cord and L1 DRG from control and SMA mice at the indicated post-natal days. (C) Strategy for labeling motor neurons and proprioceptive neurons of the motor circuit by CTb-488 injection in the iliopsoas muscle. (D) Confocal image of CTb-488-labelled iliopsoas motor neurons and DRG neurons from a control mouse. Scale bar, 100 μm. (E) Confocal image showing co-localization of CTb-488 (green) and ChAT (red) in motor neurons from the ventral horn of a CTb-injected mouse. Scale bar, 50 μm. (F) Confocal image showing co-localization of CTb-488 (green) and parvalbumin (blue) in proprioceptive neurons from the DRG of a CTb-injected mouse. Scale bar, 20 μm. (G) RT-qPCR analysis of Stasimon U12 intron retention and mRNA levels in motor neurons and proprioceptive neurons isolated by LCM from CTb-injected control and SMA mice at P6. (H) Model for the sequence of SMN-dependent molecular events necessary for normal motor circuit function which are disrupted in SMA. See also FIG. 14.

FIG. 8. Characterization of NIH3T3 cell lines with regulated knockdown of endogenous mouse SMN. (A) Schematic representation of the lentiviral vectors used to establish NIH3T3 cell lines with inducible RNAi knockdown of endogenous mouse SMN (NIH3T3-Smn_{RNAi}) as well as cell lines with inducible RNAi knockdown of endogenous mouse SMN and constitutive expression of RNAi-resistant human SMN (NIH3T3-SMN/Smn_{RNAi}). (B) RT-qPCR analysis of mouse SMN mRNA expression in wild-type NIH3T3 (Control), NIH3T3-Smn_{RNAi} and NIH3T3-SMN/Smn_{RNAi} cells cultured without (blue bars) or with (red bars) Dox for 5 days. RNA levels in Dox-treated cells are expressed relative to those in untreated cells. Data are represented as mean and SEM. Dox treatment has no effect on expression of SMN mRNA in wild-type NIH3T3 cells while causes knockdown of SMN mRNA in NIH3T3-Smn_{RNAi} and NIH3T3-SMN/Smn_{RNAi} cells to a similar extent. (C) SMN deficiency decreases U1 snRNP assembly in NIH3T3 cells. Extracts from wild-type NIH3T3 (Control), NIH3T3-Smn_{RNAi} and NIH3T3-SMN/Smn_{RNAi} cells cultured without (−) or with (+) Dox for 5 days were prepared using three independent biological replicates per group and analyzed in parallel. In vitro snRNP assembly experiments were carried out with radioactive U1 snRNA and extracts from NIH3T3 cells followed by immunoprecipitation with anti-SmB antibodies, electrophoresis on denaturing polyacrylamide gels and autoradiography. In SMN-deficient NIH3T3 cells, the reduction in U1 snRNP assembly and the degree of its correction by human SMN is proportional to the levels of SMN expression relative to the uninduced state (see FIG. 1A). (D) Quantification of SMN-dependent U1 snRNP assembly defects in NIH3T3 cells. The amounts of immunoprecipitated U1 snRNA from the experiment in (C) were quantified and RNA levels in Dox-treated cells expressed relative to those in untreated cells. Data are represented as mean and SEM. (E) SMN deficiency decreases snRNP assembly of all snRNAs of the Sm class. In vitro snRNP assembly experiments were carried out with each of the indicated snRNAs and extracts from NIH3T3-Smn_{RNAi} cells cultured without or with Dox for 5 days as in (C). The amount of each snRNA immunoprecipitated from snRNP assembly reactions with extracts from Dox-treated cells were quantified and expressed relative to those from untreated cells. Data are represented as mean and SEM. (F) Expression and splicing analysis of U12 intron-containing mouse genes in NIH3T3 cells. RT-PCR analysis of the indicated U12 intron-containing genes in wild-type NIH3T3 (Control) and NIH3T3-Smn_{RNAi} cells cultured without (−) or with (+) Dox for 5 days. Genes and exons monitored by PCR are indicated on the left. Schematics of spliced and intron-containing mRNAs are shown on the right. Callout lines highlight the position of U12 introns. The −RT lanes correspond to RT-PCR reactions lacking reverse transcriptase. (G) Expression and splicing analysis of U2 intron-containing mouse genes in NIH3T3 cells. RT-PCR analysis of the indicated genes was carried out as in (F). The Drosophila homologs of Mgat1 (CG13431), Sf3a1 (CG16941), Znf830 (CG11839) and Zrsr2 (CG3294) are U12 intron-containing genes, three of which (CG13431, CG16941 and CG11839) are affected by SMN deficiency in smn mutant third-instar larvae.

FIG. 9. Specificity of the effects of SMN deficiency in NIH3T3 cells. (A) Analysis of U2 intron splicing in mouse SMN target genes with U12 introns. RT-PCR analysis of U2 splicing in the indicated genes was carried out in wild-type NIH3T3 (Control) and NIH3T3-Smn_{RNAi} cells cultured without (−) or with (+) Dox for 5 days. Genes and exons monitored by PCR are indicated on the left. Schematics of spliced and intron-containing mRNAs are shown on the right. Gapdh was used for RNA calibration across samples. The −RT lanes correspond to RT-PCR reactions lacking reverse transcriptase. (B) Dox has no effects on proliferation of NIH3T3 cells. Equal numbers of wild-type NIH3T3 cells were plated and cultured either with or without Dox for the indicated number of days. Cell number was determined at each time point. (C) RNAi-resistant human SMN corrects cell proliferation defects caused by depletion of mouse SMN in NIH3T3 cells. Equal numbers of NIH3T3-SMN/Smn_{RNAi} cells were plated and cultured either with or without Dox for the indicated number of days. Cell number was determined at each time point. (D) Serum deprivation decreases proliferation of NIH3T3 cells. Equal numbers of wild-type NIH3T3 cells were plated and cultured in the presence of either 10% FBS or 2% FBS for the indicated number of days. Cell number was determined at each time point. (E) Serum deprivation does not decrease SMN levels in NIH3T3 cells. Western blot analysis of wild-type NIH3T3 cells cultured in the presence of 2% FBS for the indicated number of days. (F) Effect of serum deprivation on SMN-dependent U12 splicing events. RT-qPCR analysis of U12 intron retention in Clcn7, Parp1, Tspan31 and Tmem41b mRNAs. Red bars show RNA levels in NIH3T3-Smn_{RNAi} cells cultured for 3 days with Dox relative to untreated cells (dotted line). Blue bars show RNA levels in wild-type NIH3T3 cells cultured for 3 days with 2% FBS relative to cells grown with 10% FBS (dotted line). Data are represented as mean and SEM. (G) Effect of serum deprivation on SMN-dependent U12 splicing events. RT-qPCR analysis of the levels of aberrantly spliced Tmem41b and exon-skipped Clcn7 mRNAs. Red bars show RNA levels in NIH3T3-Smn_{RNAi} cells cultured for 5 days with Dox relative to untreated cells (dotted line). Blue bars show RNA levels in wild-type NIH3T3 cells cultured for 5 days with 2% FBS relative to cells grown with 10% FBS (dotted line). Data are represented as mean and SEM.

FIG. 10. Expression and splicing analysis of putative U12 intron-containing genes in Drosophila. (A) RT-PCR analysis of bioinformatically-predicted U12 intron-containing genes whose expression is not affected in Drosophila smn mutants. Equal amounts of total RNA from control, smn^{73Ao} and U6atac^{K01105} third-instar Drosophila larvae were used. Genes and exons monitored by PCR are indicated on the left. Schematics of spliced and intron-containing mRNAs are shown on the right. Callout lines highlight the position of U12 introns. The −RT lanes correspond to RT-PCR reactions lacking reverse transcriptase. (B) RT-PCR analysis of Drosophila genes that contain only U2 introns. CG7939 (RpL32) was used for RNA calibration across samples in both (A) and (B). The mouse homologs of CG8594 (Clcn7), CG8545 (Noll), CG40411 (Parp1), CG6335 (Harsl) and CG8454 (Vps16) are U12 intron-containing genes affected by SMN deficiency in NIH3T3 cells. (C) RT-qPCR analysis of U12 intron-containing genes whose mRNA expression is affected in both U6atac^{K01105} and smn^{73Ao} mutants using total RNA from control and smn^{X7} third-instar Drosophila larvae. The mRNA levels in smn^{X7} mutants were expressed relative to control larvae. Data are represented as mean and SEM.

FIG. 11. Efficiency of RNAi-mediated knockdown of SMN target genes in Drosophila. RT-qPCR analysis of mRNA expression levels was carried out using total RNA from control and RNAi larvae. For all genes except CG16941, the effects of expression of UAS-RNAi constructs by a ubiquitous driver (da-Gal4) were analyzed in whole third-instar Drosophila larvae. Due to embryonic lethality of ubiquitous CG16941 RNAi, mRNA knockdown of this gene was analyzed in muscle tissue following expression of the UAS-RNAi construct with a muscle-specific driver (G14-Gal4). The mRNA levels in RNAi larvae were expressed relative to those in the corresponding controls (without Gal4 expression). Data are represented as mean and SEM.

FIG. 12. Stasimon is an evolutionarily conserved protein that is highly expressed in the CNS. (A) Alignment of Stasimon protein sequences highlights strong evolutionary conservation across species. The amino acid sequences of human STASIMON/TMEM41b (GenBank™ NP_{—}055827) and orthologs from P. troglodytes (Ensembl ID ENSPTRG00000003346), C. familiaris (GenBank™ XP_{—}851421), R. norvegicus (GenBank™ NP_{—}001012358), M. musculus (GenBank™ NP_{—}705745), G. gallus (GenBank™ NP_{—}001008469), X. tropicalis (GenBank™ NP_{—}001016955), D. rerio (GenBank™ NP_{—}001073456), C. elegans (GenBank™ NP_{—}001073456) and D. melanogaster (GenBank™ NP_{—}573225) were aligned using ClustalW2 and BOXSHADE 3.21. Black boxes highlight identical amino acids; grey boxes highlight similar amino acids. (B) Schematic representation of Stasimon protein structure showing the position of the predicted six transmembrane domains and SNARE-associated domain. (C) In situ hybridization in Drosophila embryos shows ubiquitous expression of Stasimon mRNA with remarkably high levels in both brain and ventral cord. (D) In situ hybridization in the spinal cord of wild type mice at P4 shows widespread neuronal expression of Stasimon mRNA with remarkably high levels in motor neurons and DRG neurons. Image from the Allen Brain Atlas (see internet hypertext transfer protocol for subdomain mousespinal in domain brain-map in catgoery org) (Lein et al., 2007).

FIG. 13. Specificity of Stasimon effects on motor axon development in zebrafish embryos. (A) Schematic representation of a portion of the zebrafish stasimon pre-mRNA that includes the splice junction between intron 1 and exon 2 targeted by stas MO. Splice sites (GU and AG) and branch point adenosine (A) are indicated (top panel). RT-qPCR analysis of Stasimon mRNA levels in zebrafish Tg(mnx1:GFP) embryos injected with Control MO and stas MO normalized to β actin mRNA (bottom panel). Data are represented as mean and SEM. (B) Western blot analysis of hnRNP Q protein levels in Tg(mnx1:GFP) embryos injected with Control MO and hnRNP Q MO. (C) Representative lateral view of motor axons in zebrafish Tg(mnx1:GFP) morphants injected with hnRNP Q MO. (D) Quantification of the effects of hnRNP Q deficiency on motor axon development in zebrafish. Motor axons were scored in Tg(mnx1:GFP) embryos injected with Control MO or hnrnp Q MO. Embryos were classified as severe, moderate, mild or no defects based on the severity of motor axons defects, as previously described (Carrel et al., 2006), and the percentage for each group is shown. Data are represented as mean and SEM. (E) Quantification of the effects of Stasimon overexpression on normal motor axon development in zebrafish. Motor axons were scored in Tg(mnx1:GFP) embryos injected with either control MO or STAS RNA and embryos were classified as in (D). Data are represented as mean and SEM. (F) Representative lateral view of motor axons in zebrafish Tg(mnx1:GFP) morphants co-injected with smn MO and Bcl2 RNA. (G) Quantification of Bcl2 effects on SMN-dependent motor axons defects in zebrafish. Motor axons were scored in Tg(mnx1:GFP) embryos injected with smn MO or smn MO together with Bcl2 RNA and embryos were classified as in (D). Data are represented as mean and SEM. (H) Western blot analysis of Smn protein levels in Tg(mnx1:GFP) embryos injected with Control MO as well as smn MO either in the presence or in the absence of co-injected STAS RNA and Bcl2 RNA.

FIG. 14. Effects of SMN deficiency on Stasimon U12 splicing and mRNA expression of in SMA mice. (A) Schematic of the portion of Stasimon pre-mRNA from exon 3 to exon 5 including the splice sites and introns are shown at the top. Green lines indicate the aberrant splicing event due to activation of a cryptic 5′ splice site in exon 3 that is caused by SMN deficiency. Schematics of intron-containing and aberrantly spliced Stasimon mRNAs are shown at the bottom. Arrows represent the primers used for RT-qPCR. (B) RT-qPCR analysis of the aberrantly spliced Stasimon mRNA in the brain and kidney from control and SMA mice at the indicated post-natal days. Data are represented as mean and SEM. (C) RT-qPCR analysis of Stasimon U12 intron retention in the brain and kidney from control and SMA mice at the indicated post-natal days. Data are represented as mean and SEM. (D) RT-qPCR analysis of Stasimon mRNA expression in the brain, kidney, spinal cord and L1 DRG from control and SMA mice at the indicated post-natal days. Data are represented as mean and SEM. (E) Representative epifluorescence and bright-field images of the ventral spinal cord before and after LCM of CTb-488 labeled motor neurons. (F) Representative epifluorescence and bright-field images of the DRG before and after LCM of CTb-488 labeled proprioceptive neurons. (G) RT-qPCR analysis of ChAT and Parvalbumin expression validating the identity of the collected CTb-488⁺ LCM neurons in the spinal cord as motor neurons and in the DRG as proprioceptive neurons. Data are represented as mean and SEM. The levels of Stasimon mRNA in LCM iliopsoas motor neurons relative to LCM iliopsoas proprioceptive neurons at P6 were similar.

FIG. 15. A flow chart that illustrates an example method for detecting, monitoring and treating SMA, according to an embodiment.

FIG. 16. Effects of expression of a Rash cDNA in smn mutant on locomotion speed.

FIG. 17. Efficient in vivo transduction of motor circuit neurons with AAV9 vectors. (A) Schematic of the self-complementary AAV9 (scAAV) vectors used for the expression of GFP, human SMN or human STASIMON in SMAA7 mice. (B) Western blot analysis of brain tissue from wild-type (Control) and SMAA7 SMA mice injected with the indicated scAAV9 vectors. Mice were injected ICV at P1 with 1×10¹¹ gc of each vector and tissue collected at P11. Note that scAAV9-STAS injection does not increase SMN levels in SMAA7 SMA mice. (C) Immunohistochemistry and confocal microscopy analysis demonstrating robust GFP expression in motor circuit neurons, including parvalbumin (Pvb)⁺ proprioceptive neurons in the DRG and ChAT motor neurons in the spinal cord of scAAV9-GFP injected mice at P11. (D) Percentage GFP-expressing proprioceptive neurons and motor neurons following scAAV9-GFP injection.

FIG. 18. AAV-SMN restores normal U12 splicing of Stasimon mRNA in SMAA7 SMA mice. RT-qPCR analysis of aberrant U12 splicing of Stasimon mRNA in the DRG and spinal cord of SMAD7 mice injected with scAAV9-GFP and scAAV9-STAS relative to wild-type (Normal) mice. Mice were injected at P1 and tissue collected at P11. Note that SMN restores normal U12 splicing of Stasimon, which correlates with phenotypic correction.

FIG. 19. AAV-STAS improves the SMA phenotype in SMAΔ7 SMA mice. Representative image of SMAΔ7 SMA mice injected with scAAV9-STAS and scAAV9-GFP at P14. Note that STAS-injected mice stand properly on the four limbs and are well-groomed, larger and healthier than GFP-injected controls.

FIG. 20. AAV-STAS improves weight gain in SMAD7 mice. Weight gain in wild-type (Control) and SMAΔ7 SMA mice injected with the indicated scAAV9-vectors. Note that STAS-injected SMAΔ7 SMA mice gain much more weight than GFP-injected controls. The effect of STAS on weight is comparable to if not better than that of SMN-injected mice until P12, after which it progressively declines.

FIG. 21. AAV-STAS increases lifespan in SMAA7 SMA mice. Kaplan-Meier survival plot of wild-type (Control) and SMAΔ7 SMA mice injected with the indicated scAAV9-vectors. Note that STAS injection leads to a 25% increase in the lifespan of SMAA7 SMA mice (GFP=median survival=17.5 days; STAS median survival=17.5 days; SMN median survival=40 days).

Table 1. U12 intron-containing mouse genes analyzed in this study.

Table 2. Drosophila genes with bioinformatically predicted U12 introns analyzed in this study.

Table 3. Primers and probes used in this study.

Table 4: Diagnostic Genes.

### DEFINITIONS

The following terms as used herein have the corresponding meanings given here.

“Motor neuron diseases (MNDs)” means a group of progressive neurological disorders that destroy motor neurons, the cells that control essential voluntary muscle activity such as speaking, walking, breathing, and swallowing. Included among MNDs are: spinal muscular atrophy (SMA) and ALS. Motor system diseases include diseases involving proprioception such as Ehlers-Danlos Syndrome, for the purpose of these inventions MND and Motor system diseases are used interchangeably.

“Significantly higher or significantly lower” means a respective statistically significant difference.

A “U12 intron” means the portion of certain precursor mRNAs that is specifically removed by the U12 spliceosome which is formed from U12 small nuclear (snRNA), together with U4_{atac}/U6_{atac}, U5, and U11 snRNAs and associated proteins. This spliceosome processes a divergent class of low-abundance pre-mRNA introns. The splicing reaction is essentially similar to that of the major class of introns, and the snRNAs play analogous roles: U11 base pairs with the 5′ splice sites; U12 base pairs with the branch point sequence near the 3′ splice site; and U4_{atac} and U6_{atac} provide analogous functions during the spliceosome assembly and activation of the spliceosome. Consensus sequences define the 5′ and 3′ splice sites of U12 type introns: 5′^{G}_{A}UAUCCUUU . . . PyA^{G}_{C}3′. Although originally discovered for the presence of unusual AT . . . AC di-nucleotides at the splice site, most U12-dependent introns actually have the GU . . . AG termini. They have a highly conserved branch point (UCCUUPuAPy), which pairs with U12 snRNP, and lack a polypyrimidine tract (Yn). These differences distinguish the major and minor classes of introns. The minor class of introns is called U12-dependent introns.

“Aberrant splicing” events as used herein, means events that are mediated by the U2 spliceosome and characterized by the use/activation of an upstream cryptic (normally silent) 5′ splice site that is spliced to a downstream U2-dependent 3′ splice site, thereby producing mRNA that is atypical.

“Alternative splicing” events as used herein, means events that are mediated by the U2 spliceosome and characterized by the skipping of the two exons flanking the U12 intron. Exon skipping is an example of an alternative splicing event. Thus, the main difference between alternative and aberrant splicing as used herein, is that a canonical U2-dependent 5′ splice site is used as the donor in the case of alternative splicing, while a cryptic U2-dependent 5′ splice site in the case of aberrant splicing.

A “twintron” (intron-within-intron) as used herein refers to the presence of the U12 intron within a U2 intron in the Stasimon pre-mRNA. The excision of these introns can be mutually exclusive or sequential, in the latter case the U12 introns is removed before the U2 intron.

A “spliceosome” means a large macromolecular RNA-protein complex formed as a result of snRNPs involved in splicing together with many additional proteins. This complex of snRNPs and proteins removes introns from a transcribed pre-mRNA (hnRNA). Such a process is generally referred to as splicing. Each spliceosome is composed of five small nuclear RNAs (snRNA), and a range of associated protein factors. When these small RNAs are combined with the protein factors, they make an RNA-protein complex called a snRNP. The snRNAs that make up the nuclear spliceosomes are named U1, U2, U4, U5, and U6 (for the U2 spliceosome) and U11, U12, U5, U4atac, U6atac (for the U12 spliceosome), and participate in several RNA-RNA and RNA-protein interactions. The RNA component of the small nuclear ribonucleoprotein, or snRNP, is rich in uridine (the nucleoside analog of the uracil nucleotide).

“snRNPs (pronounced “snurps”), or small nuclear ribonucleoproteins” mean RNA-protein complexes that combine with unmodified pre-mRNA and various other proteins to form a spliceosome, a large RNA-protein molecular complex upon which splicing of pre-mRNA occurs. The action of snRNPs is essential to the removal of introns from pre-mRNA, a critical aspect of post-transcriptional modification of RNA, occurring only in the nucleus of eukaryotic cells. The two essential components of snRNPs are protein and RNA molecules. The RNA found within each snRNP particle is known as small nuclear RNA, or snRNA, and is usually about 150 nucleotides in length. The snRNA component of the snRNP gives specificity to individual introns by “recognizing” the sequences of critical splicing signals at the 5′ and 3′ ends and branch site of introns. The snRNA in snRNPs is similar to ribosomal RNA in that it directly incorporates both a catalytic and a structural role.

“5′ UTR untranslated region” or “5′UTR” means a region of a messenger ribonucleic acid (mRNA) molecule encoding a protein that is not translated into protein. Eukaryotic mRNA has several untranslated regions: the 5′ untranslated region, 3′ untranslated region, 5′ cap and poly-A tail. The 5′ UTR can contain elements for controlling gene expression by way of regulatory elements. It begins at the transcription start site and ends one nucleotide (nt) before the start codon (usually AUG) of the coding region. In prokaryotes, the 5′ UTR usually contains a ribosome binding site (RBS), also known as the Shine Dalgarno sequence (AGGAGGU). The 5′ UTR has a median length of ˜150 nt in eukaryotes, but can be as long as several thousand bases.

“Gene” means a locatable region of genomic sequence, corresponding to a unit of inheritance, which is associated with regulatory regions, transcribed regions, and or other functional sequence regions. The segment of DNA in a gene is involved in producing a polypeptide chain. Specifically, a gene includes, without limitation, regions preceding and following the protein-coding mRNA region, such as the promoter and 3′-untranslated region, respectively, as well as intervening sequences (introns) between individual coding segments (exons).

“Biological sample” means any biological specimen obtained from a subject. Preferred biological samples for monitoring levels of SMN, Stasimon, or aberrantly or alternatively spliced mRNAs as described herein. Preferred biological samples for monitoring these levels of Stasimon and Stasimon mRNAs include fibroblasts, blood, serum, muscle and cerebrospinal fluid.

A “subject” means any mammal including, e.g., humans, dogs, cows, horses, kangaroos, pigs, sheep, goats, cats, mice, rabbits, rats, and transgenic non-human animals. Synonyms used herein include “patient,” “subject” and “animal.”

“Therapeutic agent” means an agent that is used to treat SMN deficiency or a related disease such as SMA, particularly the Stasimon protein or gene or mRNA encoding Stasimon or biologically active fragments or variants thereof.

“Biomarker” means a molecule that can be detected in a biological sample taken from a subject, which molecule is an indicator of the presence of a disease state such as SMA or SMN deficiency. In the context of the present invention, Stasimon protein or gene or mRNA encoding Stasimon is a biomarker of SMA or SMN deficiency.

“Treating” means taking steps to obtain beneficial or desired results, including clinical results, such as alleviating or ameliorating one or more symptoms of a disease; diminishing the extent of disease; delaying or slowing disease progression; ameliorating and palliating or stabilizing a metric (statistic) of disease. “Treatment” refers to the steps taken.

“Therapeutically effective amount” means an amount of a therapeutic agent which achieves an intended therapeutic effect in a subject, e.g., eliminating or reducing the severity of a disease or set of one or more symptoms. The full therapeutic effect does not necessarily occur by administration of one dose and may occur only after administration of a series of doses. Thus, a therapeutically effective amount may be administered in one or more administrations.

### DETAILED DESCRIPTION

In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention. Specific embodiments are described in the above Summary of the Invention. Some embodiments of the invention are described below in the context of certain models for the disease SMA and generally for MND. However, the invention is not limited to this context. In other embodiments gene targets that are altered by SMN are used for the detection or monitoring or treatment of the disease in other animals, including humans.

SMA is a motor neuron disease caused by deficiency of the ubiquitous SMN protein. The SMN protein forms a macromolecular complex whose only defined activity is in the biogenesis of small nuclear ribonucleoproteins (snRNPs) of the Sm-class, essential components of the RNA splicing machinery. Spliceosomal snRNPs comprise two distinct classes. While most eukaryotic introns (˜99%) are processed by the major U2-dependent spliceosome, only a small proportion of introns (˜1%) are processed by the minor U12-dependent spliceosome. Importantly, SMN deficiency changes the snRNP profile of tissues in a non-uniform manner and has been reported to preferentially reduce accumulation of minor snRNPs (Gabanella et al., 2007; Zhang et al., 2008).

It has been discovered that SMN regulation of splicing is essential for motor circuit function in vivo. Reduced SMN levels lead to altered U12 splicing and decreased expression of a discrete set of U12 intron-containing genes in both mammalian cells and Drosophila larvae. Using Drosophila SMN mutants and mouse cells incapable of making functional SMN (the NIH3T3-SmnRNAi cell line), it has now been shown that SMN-dependent U12 splicing events are critical in the regulation of motor circuit activity. Specifically, it has been discovered for the first time that there is a direct link between SMN deficiency-induced defective splicing of critical neuronal genes having a U12 intron and motor circuit dysfunction, establishing a molecular framework for the selective pathology of SMA as well as other motor neuron diseases.

SMN-deficiency in both mammalian cells and Drosophila larvae was discovered to perturb U12 splicing in mRNAs transcribed from a subset of U12 intron-containing genes. Table 1 lists 28 U12 intron-containing genes tested in mice and Table 2 indicates all the bioinformatically-predicted U12 intron-containing genes in Drosophila. 7 genes were identified that both a) contained a U12 intron which is also conserved in humans and (b) had reduced expression in SMN-deficient mouse NIH3T3 cells. Of these genes, 3 were also affected in Drosophila smn mutants. These 7 genes are referred to as the MND “diagnostic genes,” (listed in Table 4) and the proteins they encode are referred to as “diagnostic proteins. ” The seven diagnostic genes are Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1. Two novel genes are included in this list that encode proteins for which the function has just been discovered. These novel genes and proteins are named “Stasimon” and “Rashomon.” Stasimon encodes an evolutionarily conserved transmembrane protein that is required for normal neurotransmitter release by motor neurons in Drosophila and motor axon outgrowth in zebrafish, and Rashomon is a novel SMN-dependent gene that encodes a protein required for normal locomotion.

Although multiple genes are affected by SMN deficiency, restoration of Stasimon levels alone rescued key motor neuron defects in both invertebrate and vertebrate models of SMA, establishing that altered expression of individual genes can account for specific aspects of motor circuit dysfunction in vivo. Restoration of Stasimon expression alone in the motor circuit corrected defects in neuromuscular junction transmission and improved muscle growth in Drosophila SMN mutants and aberrant motor neuron development in SMN-deficient zebrafish. Decreased expression of Stasimon in cholinergic neurons accounted for the dysfunction of neurotransmitter release at the NMJ and contributed to defects of muscle growth in Drosophila smn mutants. Because the highly conserved Stasimon restored normal cholinergic motor circuit function and muscle growth in the Drosophila and zebrafish models of SMA, certain embodiments are directed to the therapeutic use of Stasimon in treating SMA and other motor neuron diseases associated with reduced Stasimon, or MND generally. The Stasimon gene can be administered as gene therapy, or alternately the Stasimon protein can be administered in therapeutically effective amounts.

Homologs of Stasimon are found in humans, mice and zebrafish in addition to other species, and display a remarkably high degree of amino acid conservation although their function was unknown until now. Because of this sequence homology, the role of Stasimon in the Drosophila, mouse and zebrafish animal models can be extrapolated to humans. “Tmem41b” is the current name for mouse and human Stasimon. The DNA sequence of the Drosophila Stasimon—CG8408 gene is at available at http://www.ncbi.nlm.nih.gov/nucleotide/24642815. The amino acid sequence of Stasimon protein in Drosophila is available at http://www.ncbi.nlm.nih.gov/protein/18859941. The human Stasimon homolog gene Tmem4lb is available at http://www.ncbi.nlm.nih.gov/nuccore/28278156. The amino acid sequence of Tmem41b protein in humans is available at http://www.uniprot.org/uniprot/Q5BJD5. The genes for the diagnostic proteins are described in Table 4.

The novel SMN-dependent gene Rashomon is required for normal locomotion as was shown in vivo in Drosophila larvae. Expression of the Rashomon gene, previously CG33108 in Drosophila, is reduced by SMN depletion, and both knockdown and mutants of CG33108 caused a locomotion phenotype similar to that of SMN mutants. Because the function of the gene CG33108 was previously unknown, it has now been renamed Rashomon—abbreviated as rash. Certain embodiments are directed to the therapeutic use of Rashomon in treating SMA and other motor neuron diseases generally by administering the gene via gene therapy, or alternately the Rashomon protein can be administered in therapeutically effective amounts. MND and SMA in particular can be treated by gene therapy using any of the diagnostic genes, or combinations thereof.

In another embodiment of the invention, a diagnosis of MND such as SMA can be made in a subject having a symptom of an MND if a biological sample from the subject indicates reduced expression of one or more diagnostic genes, such as stasimon and Rashomon, compared to normal controls. The under expression of one or more of these diagnostic genes confirms that a subject with a symptom of MND has the disease, can be determined assessing either the level of one or more diagnostic proteins or normally spliced mRNAs encoding the one or more diagnostic proteins in a biological sample from the subject, wherein the diagnostic protein or mRNA level would be significantly below normal in a subject having MND. Because mis-spliced mRNAs are rapidly cleared, the most useful test for MND is determining the level of normal mRNA encoding one or more diagnostic proteins. A subject thus diagnosed as having an MND can be treated by administering therapeutically effective amounts of one or more of the under expressed diagnostic proteins. Or, treatment can occur by gene therapy providing a gene encoding a diagnostic protein, preferably Stasimon or Rashomon, or more than one gene. Data is also provided showing that AAV-Stasimon delivery in vivo in SMA mice partially restored motor neuron function, increases weight gain and prolongs life.

Certain embodiments are directed to an AAV comprising a gene encoding one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1. Other embodiments are directed to pharmaceutical formulations comprising one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, especially those formulated to enhance uptake by the brain, spinal cord or motor neurons.

In another embodiment of the invention, a diagnosis of MND can be made in a subject having a symptom of an MND if a biological sample from the subject indicates reduced expression of one or more diagnostic proteins or mRNA encoding them compared to normal levels.

Because both Stasimon and Rashomon are important proteins for motor neuron function, certain other embodiments are directed to combination therapy treatment for MND and SMA by administering genes for both Rashomon and Stasimon via gene therapy, or by administering therapeutically effective amounts of Stasimon and Rashomon proteins either together in a single formulation or sequentially to optimize efficacy. Still other embodiments are directed to pharmaceutical formulations comprising Stasimon, or Rashomon, or both.

In another embodiment the level of Stasimon protein (or any of the other diagnostic proteins) or of normally spliced mRNA encoding Stasimon protein (or any of the other diagnostic proteins) is used to monitor the progression of an MND such as SMA, or the response of a subject with MND such as SMA to treatment (for example treatment with gene therapy introducing a normal SMN gene). In an embodiment a biological sample is taken from a subject being treated for MND or SMA, at various times during treatment, including before treatment, if possible, and after treatment. If the level of Stasimon protein or of normally spliced mRNA encoding Stasimon protein is significantly higher after treatment than before treatment, then it is determined that the treatment is efficacious and should be continued. However, if the level is significantly reduced then it is determined that the treatment is not efficacious and should be discontinued. The same conclusion is reached if the level of unspliced (i.e. U12 intron-containing) and/or aberrantly spliced mRNA is significantly increased compared to the corresponding level in normal controls.

### Overview

RNA splicing is a fundamental regulatory mechanism of eukaryotic gene expression that is crucial in the nervous system. Mutations in proteins involved in RNA splicing have been associated with human neurodegenerative diseases (Cooper et al., 2009). However, an unsolved conundrum is how disruption of ubiquitously expressed splicing factors can cause selective dysfunction of specific subsets of neurons. The inherited neurodegenerative disease spinal muscular atrophy (SMA) is a prominent example of this enigma.

SMA is an autosomal recessive disorder characterized by degeneration of motor neurons and atrophy of skeletal muscle caused by homozygous inactivation of the Survival Motor Neuron 1 (SMN1) gene (Lefebvre et al., 1995). The nearly identical SMN2 gene is unable to compensate for the loss of SMN1 as it produces low levels of functional SMN protein. Consistent with human pathology, in both invertebrate and vertebrate animal models low levels of SMN are sufficient for normal function of most cell types but not of motor neurons (Burghes and Beattie, 2009). However, the mechanisms that link ubiquitous SMN deficiency to selective neuronal dysfunction remain unclear.

The SMN protein forms a macromolecular complex whose only defined activity is in the biogenesis of small nuclear ribonucleoproteins (snRNPs) of the Sm-class (Neuenkirchen et al., 2008; Pellizzoni, 2007), essential components of the RNA splicing machinery composed of an snRNA molecule, seven common Sm proteins and additional snRNP-specific proteins. The SMN complex mediates the assembly of a heptameric ring of Sm proteins around a conserved sequence of each snRNA to form the Sm core required for snRNP stability and function (Meister et al., 2001; Pellizzoni et al., 2002). Spliceosomal snRNPs comprise two distinct classes, each dedicated to the removal of different types of introns distinguished by the presence of specific conserved features in the splice sites. Most eukaryotic introns (˜99%) are processed by the major (U2-dependent) spliceosome formed by Ul, U2, U4/U6 and U5 snRNPs, while a small proportion of introns (˜1%) are processed by the minor (U12-dependent) spliceosome comprised of U11, U12, U4atac/U6atac and U5 snRNPs. Importantly, SMN deficiency changes the snRNP profile of tissues in a non-uniform manner and appears to preferentially reduce accumulation of minor snRNPs (Gabanella et al., 2007; Zhang et al., 2008). This has led to the hypothesis that genes containing U12 introns could be among the disease-relevant targets affected by SMN reduction in SMA (Gabanella et al. 2007). Consistent with this possibility is recent evidence for inefficient U12 splicing in lymphoblastoid cells from a single SMA patient (Boulisfane et al. 2011); however, no SMN-dependent U12 splicing event had previously been directly linked to the SMA phenotype.

A combination of cellular and animal models were used to reveal for the first time a direct link between SMN deficiency and aberrant U12 splicing of various genes, including a newly identified Drosophila gene named “Stasimon” (previously designated CG8408 in Drosophila and Tmem41b in vertebrates), thereby causing motor circuit dysfunction. It was found that SMN deficiency interferes with normal U12 splicing and mRNA expression of a large subset of U12 intron-containing genes including Stasimon in mammalian cells, zebrafish and Drosophila larvae. The data summarized herein, and described in detail in the Appendices, directly link defective splicing of Stasimon with essential functions in motor circuits to the phenotypic consequences of SMN deficiency, establishing a mechanistic basis for the neuronal selectivity of SMA.

### Summary of Results

The newly discovered Drosophila gene and protein designated “Stasimon” encodes an evolutionarily conserved transmembrane protein that is required for normal neurotransmitter release by motor neurons in Drosophila and motor axon outgrowth in zebrafish. “Tmem41b” is the current name for mouse and human Stasimon.

1. SMN is required for U12 splicing in mammalian cells. SMN deficiency results in both the early production and the accumulative increase of U12 intron splicing defects in a subset of genes containing this type of intron in mammalian cells.

2. SMN is required for expression of snRNAs and U12 intron-containing genes in Drosophila. SMN deficiency affects the expression of U12 intron-containing genes in Drosophila and some of these SMN-dependent U12 splicing events are conserved across evolution.

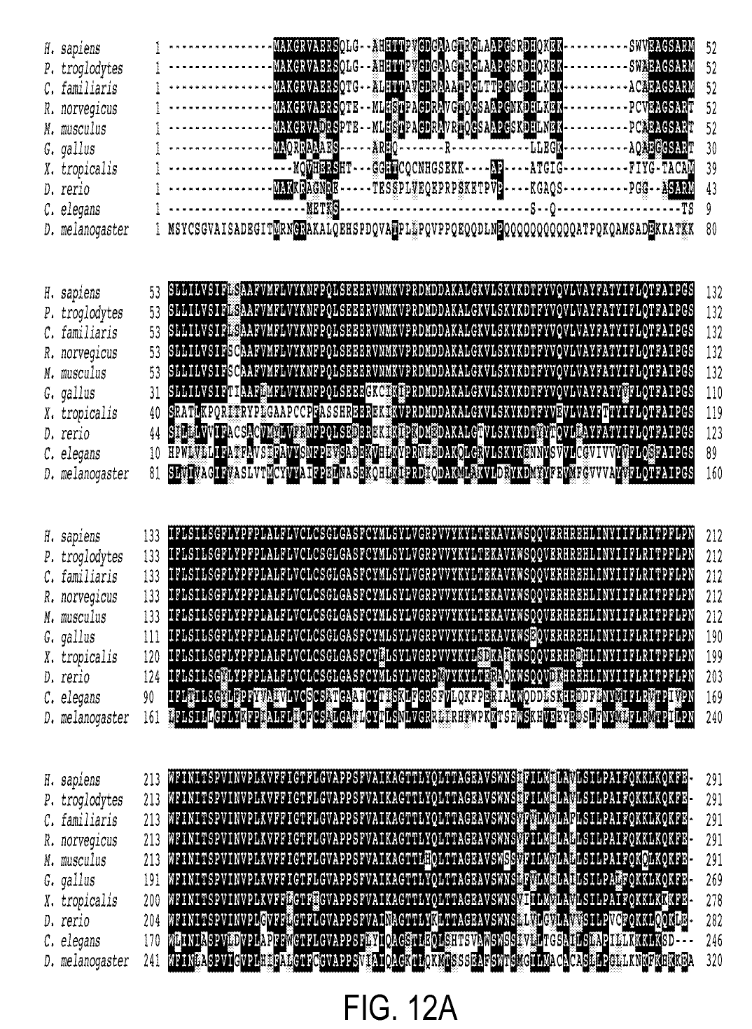

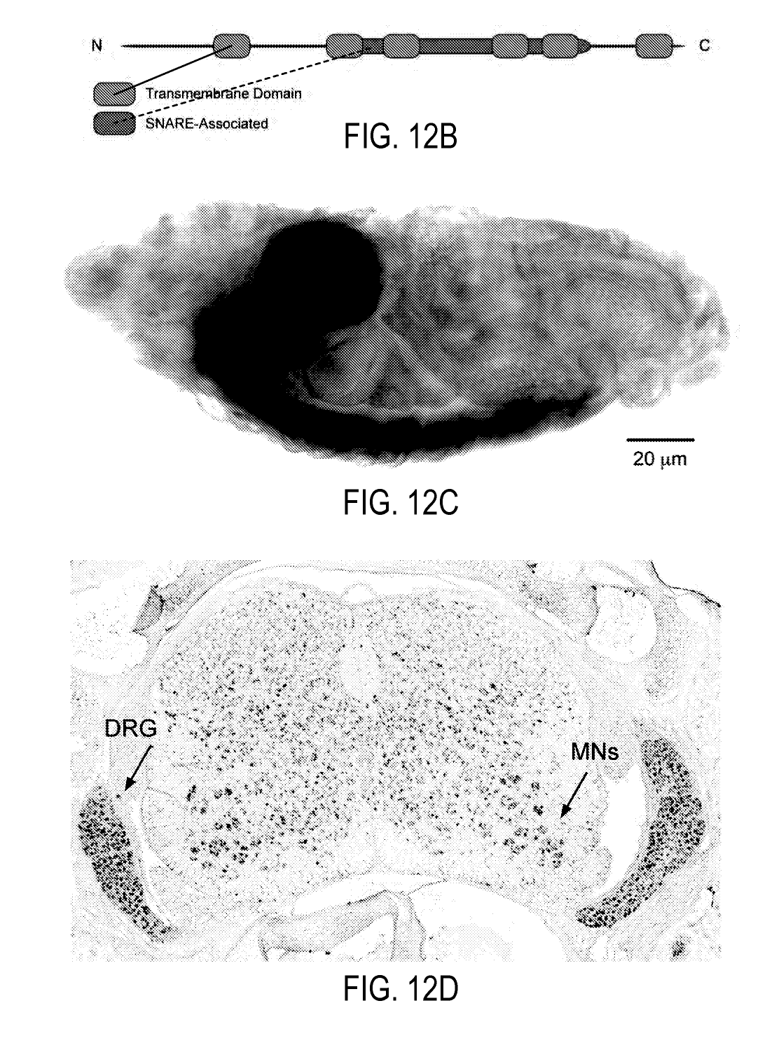

3. The Drosophila gene CG8408 and its mouse homologue Tmem41b are both U12 intron-containing genes whose expression is regulated by SMN (they are SMN target genes). It was discovered that knockdown of CG8408 in Drosophila neurons caused an electrophysiological phenotype similar to that of smn mutants. The CG8408 gene in Drosophila has been renamed stasimon (stymied in smn)—abbreviated as stas. The Stasimon protein is predicted to contain six transmembrane domains and a SNARE-associated Golgi protein domain. Homologs of Stasimon are found in humans however the function was unknown until now. Mice and zebrafish in addition to other species (FIG. 12A), and display a remarkably high degree of amino acid conservation. In situ hybridization showed strong expression of Stasimon mRNA in the nervous system of Drosophila embryos as well as in the mouse spinal cord.

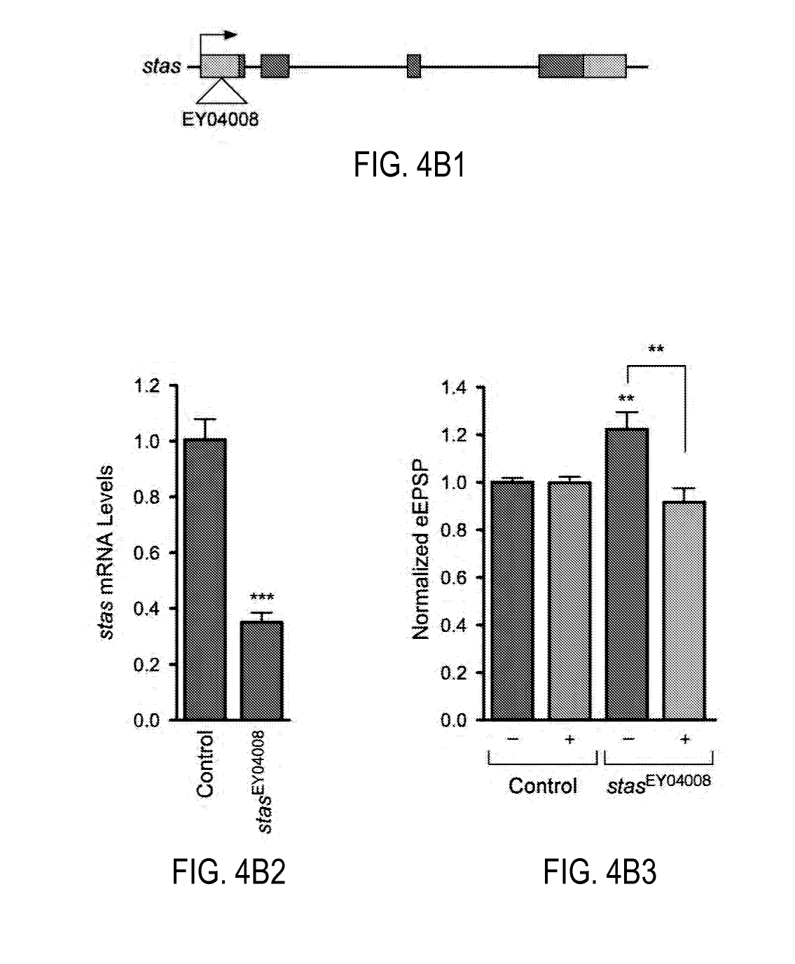

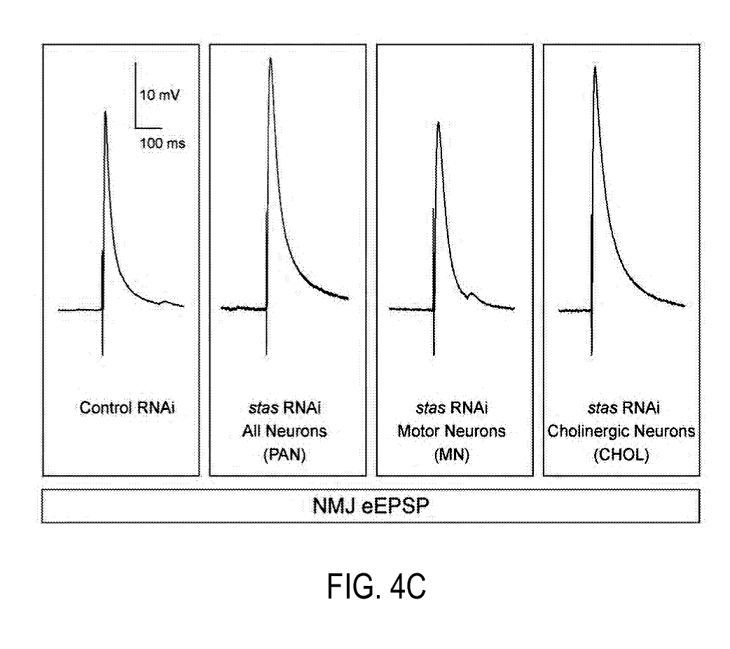

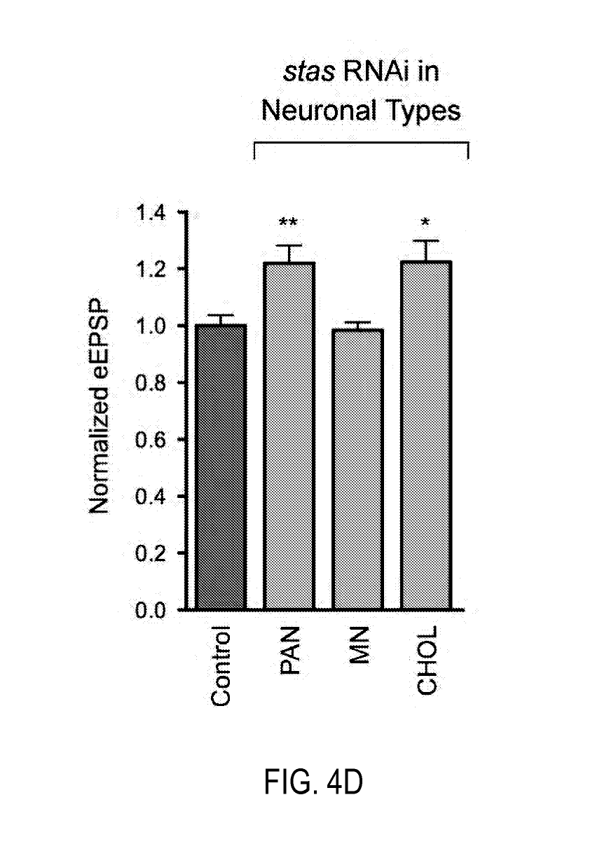

4. Stasimon is an SMN target gene required for normal synaptic transmission of Drosophila motor neurons. To confirm that Stasimon regulates neurotransmitter release at the Drosophila NMJ, a P-element mutant with an insertion in the 5′UTR of the stas gene (stas^{EY04008}) was characterized. Rescue experiments using a Gal4-driven Stasimon cDNA transgene showed that neuronal expression of transgenic Stasimon had no effect on neurotransmitter release at the NMJ of control larvae (FIG. 4B), while neuronal expression of transgenic Stasimon in stas^{EY04008} mutants fully restored NMJ eEPSP amplitudes to control levels (FIG. 4B).

5. Stasimon activity is required in cholinergic neurons for normal synaptic transmission of Drosophila motor neurons. Decreased Stasimon expression in subsets of Drosophila neurons by RNAi, perturbed the neurotransmitter release properties of motor neurons indirectly through disruption of the activity of cholinergic neurons in the motor circuit. Importantly; analogous NMJ neurotransmission defects in Drosophila smn mutants are caused by SMN deficiency in cholinergic neurons but not in motor neurons (Imlach et al., 2012).

6. Stasimon expression rescues synaptic dysfunction in Drosophila smn mutants. Restoring Stasimon expression in all neurons completely rescued the NMJ eEPSP amplitude of smn mutants to control levels, showing that Stasimon deficiency contributes to the synaptic dysfunction in Drosophila smn mutants.

7. SMN-dependent, decreased expression of Stasimon in cholinergic neurons can account for the dysfunction of neurotransmitter release at the NMJ and contributes to defects of muscle growth in Drosophila smn mutants.

8. Stasimon expression rescues SMN-dependent motor neuron defects in zebrafish. Stasimon deficiency causes motor axon outgrowth defects in zebrafish embryos similar to those induced by SMN deficiency.

9. SMN deficiency disrupts Stasimon U12 splicing and mRNA expression in the constituent neurons of the sensory-motor circuit in a mouse model of SMA.

10. SMN regulation of splicing is essential for motor circuit function in vivo. Reduced SMN levels lead to altered U12 splicing and decreased expression of a discrete set of U12 intron-containing genes in both mammalian cells and Drosophila larvae, including Stasimon, one of these SMN target genes that encodes an evolutionarily conserved transmembrane protein that is required for normal neurotransmitter release by motor neurons in Drosophila and motor axon outgrowth in zebrafish.

11. In Drosophila, Stasimon is not required cell autonomously in motor neurons but regulates the neurotransmitter release properties of motor neurons indirectly through activities in other motor circuit neurons (cholinergic neurons) that provide excitatory input to motor neurons, similar to the cellular requirement for SMN in this model (Imlach et al., 2012). Although multiple genes are affected by SMN deficiency, restoration of Stasimon levels alone rescues key motor neuron defects in both invertebrate and vertebrate models of SMA, establishing that altered expression of individual genes can account for specific aspects of motor circuit dysfunction in vivo.

12. Another newly discovered SMN-target U12 containing gene has been discovered. This gene is named Rashomon and it is an SMN-dependent gene encoding a protein required for normal locomotion.

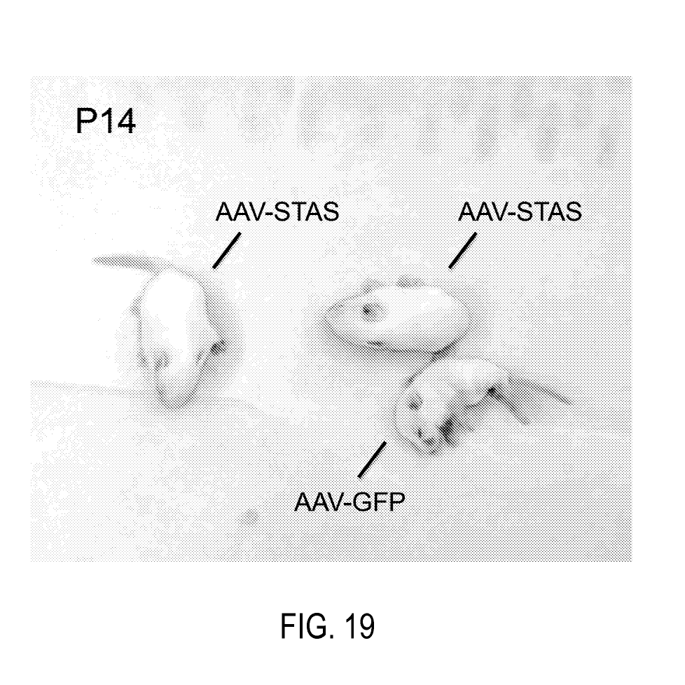

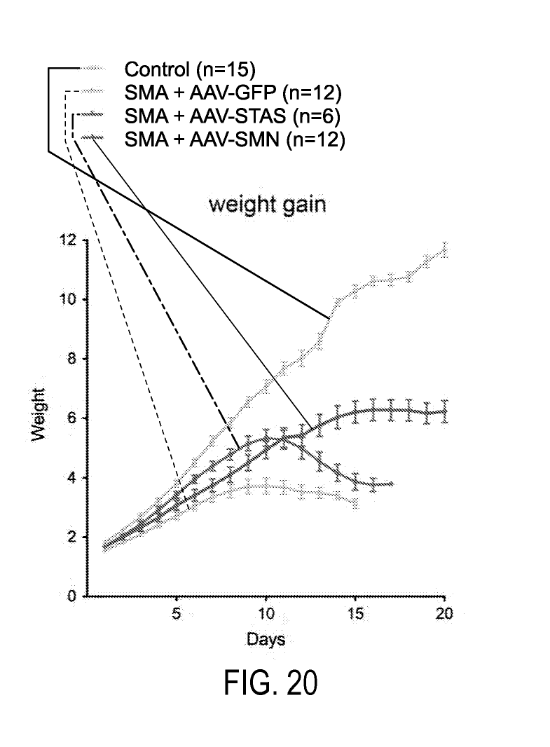

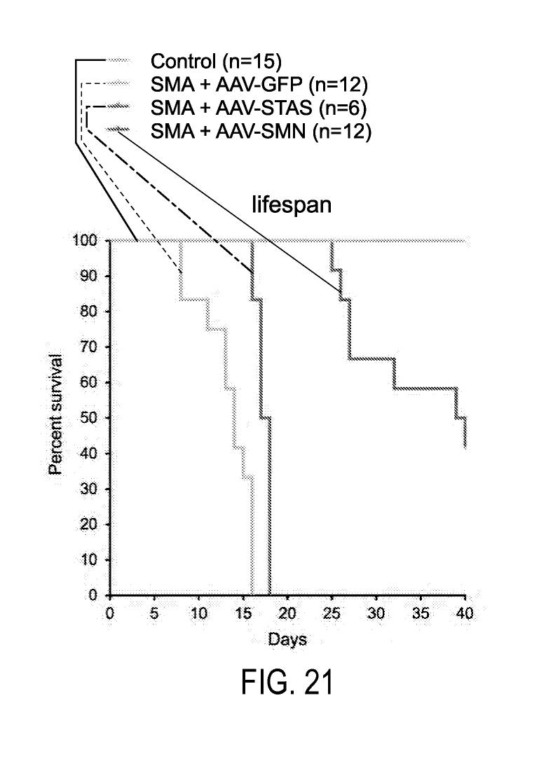

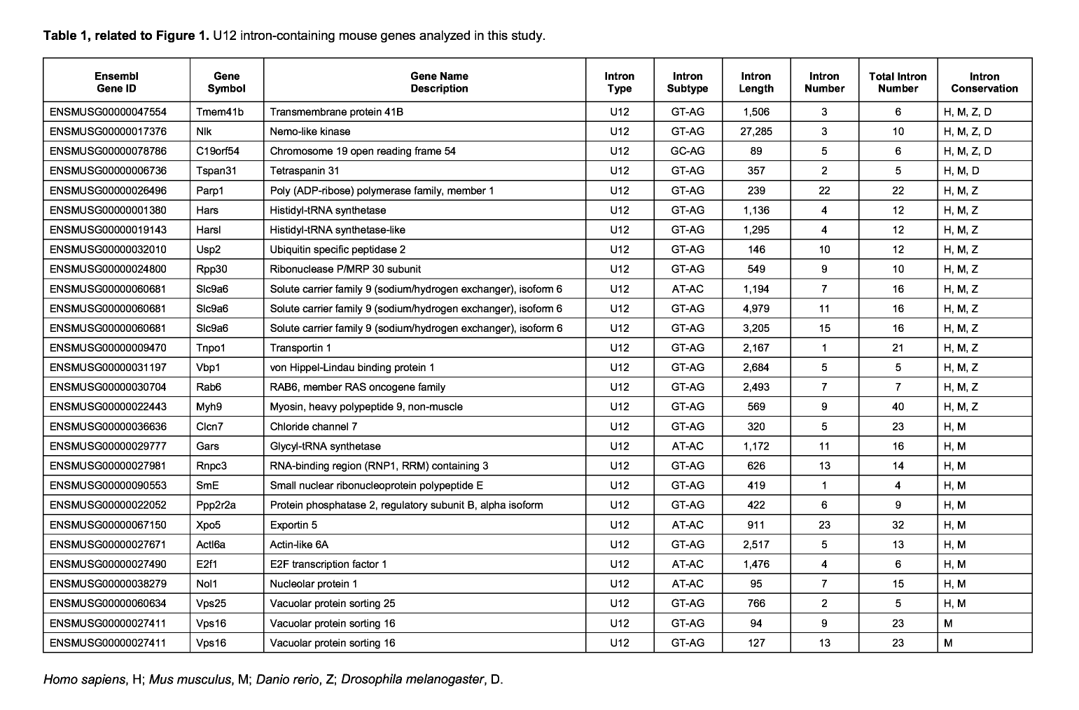

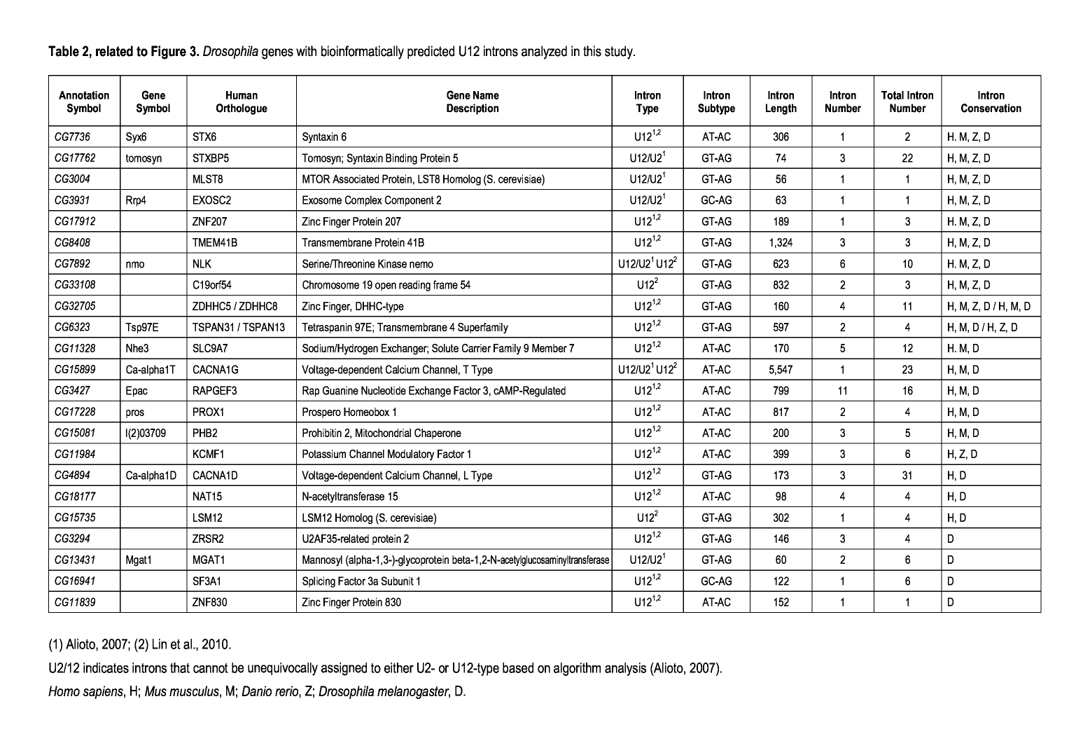

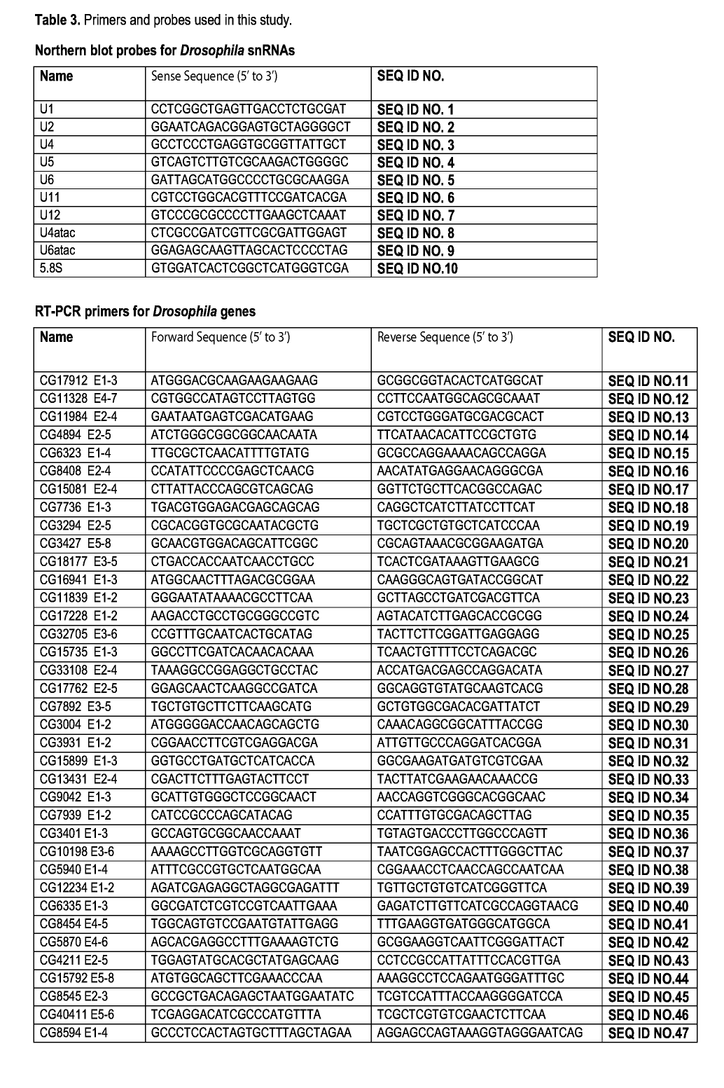

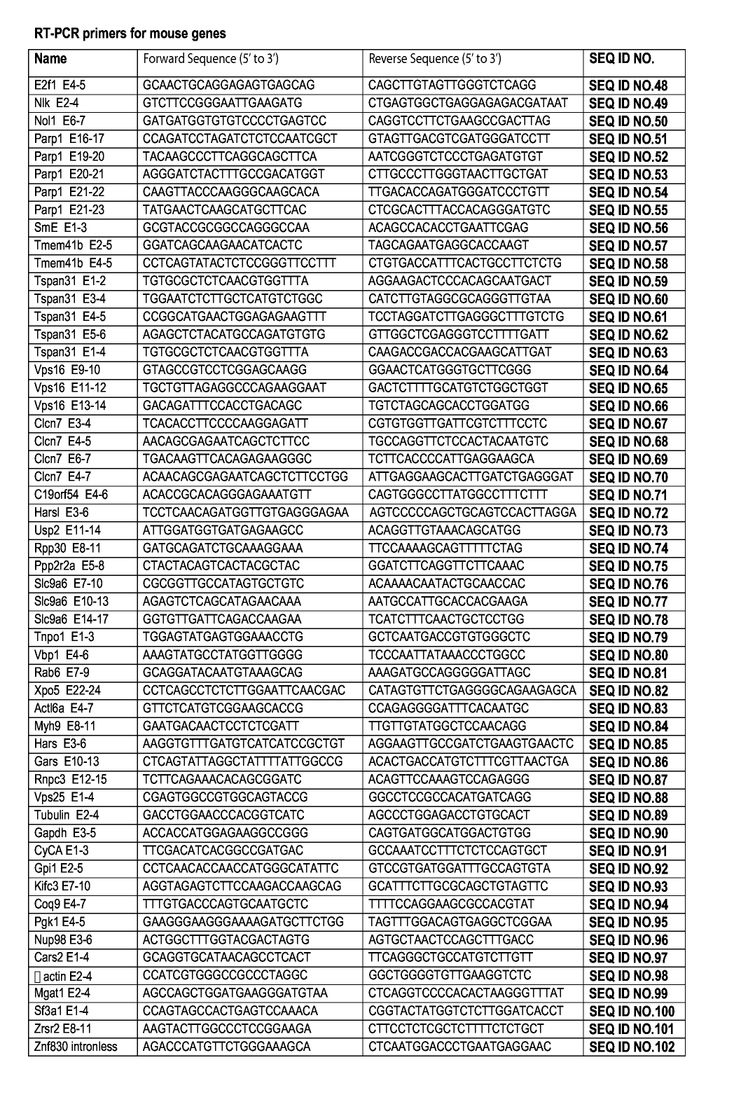

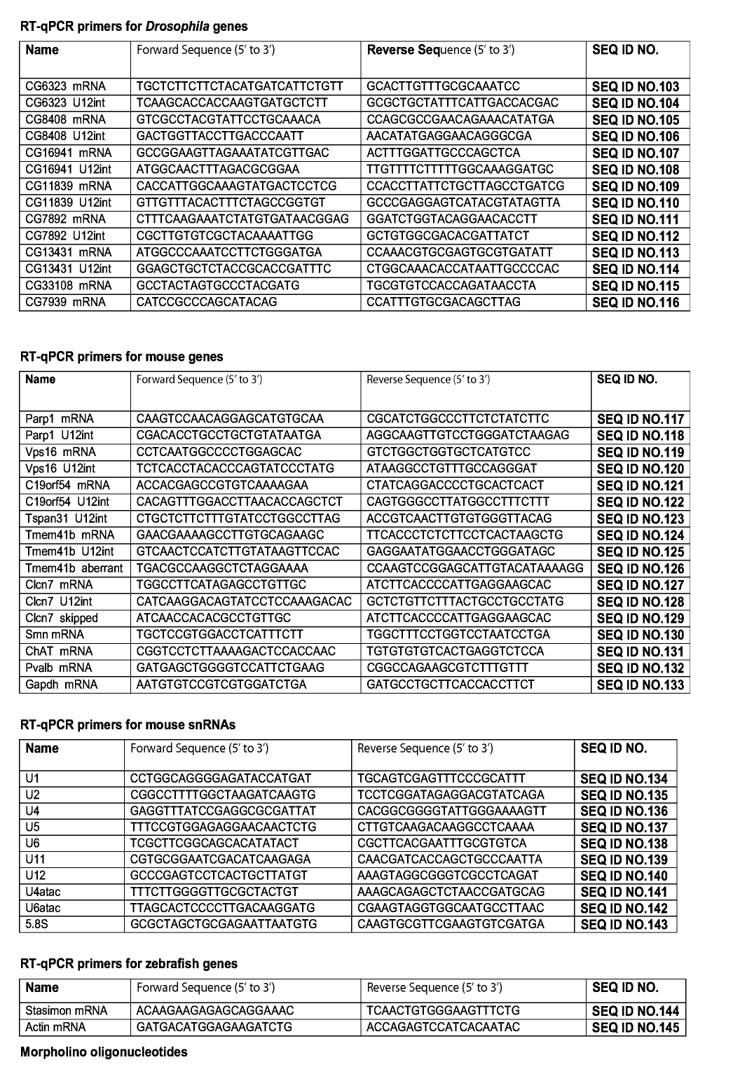

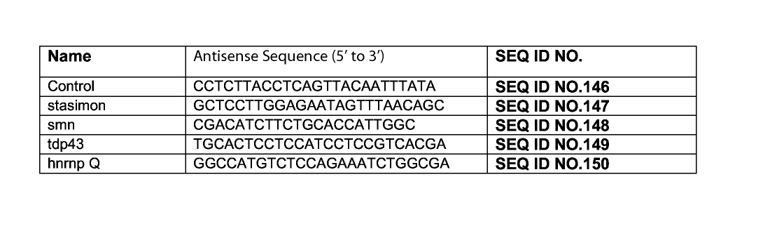

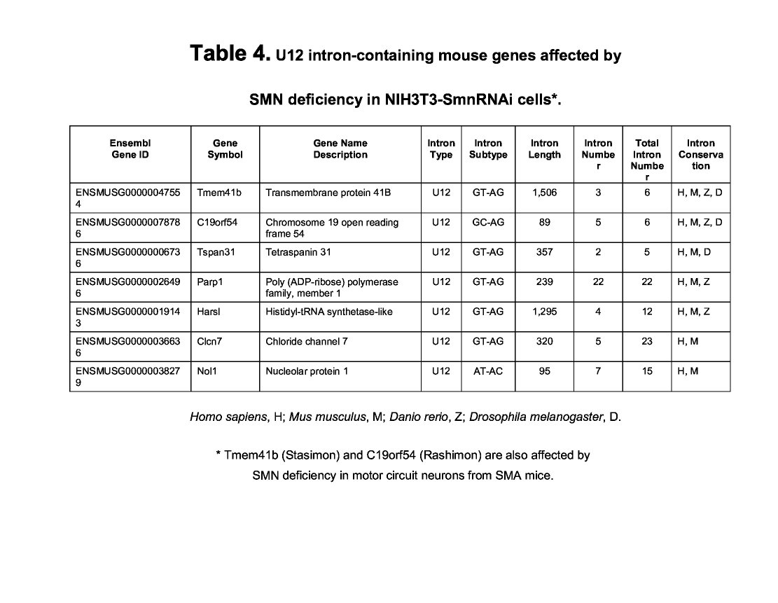

13. Example 10 describes AAV-Stasimon gene therapy in SMNΔ7 SMA mice in vivo, showing STAS-injected mice stand properly on the four limbs and are well-groomed, larger and healthier than GFP-injected controls (FIG. 19). STAS injection leads to a 25% increase in the lifespan of SMNΔ7 SMA mice (FIG. 21) and the effect of STAS on weight is comparable to if not better than that of SMN-injected mice until P12 (FIG. 20). Therefore certain embodiments are directed to methods of treating motor neuron diseases with gene therapy with stasimon.

SMN-dependent U12 splicing defects of mRNA such as are seen in Stasimon and Rashomon and the other diagnostic genes, provide a cohesive explanation for the chain of events that produce motor circuit dysfunction in SMA and other MND, and a molecular framework to understand the specific neuronal effects that result from the ubiquitous disruption of snRNP assembly by SMA and other MND.

Stasimon is a ubiquitously expressed gene with a prominent expression in the Drosophila and mouse central nervous system and encodes a highly evolutionarily conserved protein containing six transmembrane domains and a region with homology to SNARE-associated Golgi proteins. These features are consistent with a neuronal function of Stasimon in transport or docking of vesicular cargo whose impairment in neurons could disrupt neuronal activity.

The results show that restoration of Stasimon expression in cholinergic neurons is necessary and sufficient to fully rescue aberrant neurotransmitter release at the NMJs and to robustly improve muscle growth defects in SMN loss-of-function mutants, mirroring the cellular requirement for SMN in the Drosophila motor circuit. Therefore, decreased Stasimon function in cholinergic neurons directly contributes to disruption of motor circuit activity triggered by SMN deficiency and has non-cell autonomous effects in motor neurons and muscle. These findings directly link selective neuronal effects of ubiquitous SMN deficiency to defective splicing of a gene with essential functions in motor circuits. Rashomon is also incorrectly spliced as a result of SMN-deficiency, and is required for locomotor activity.

These observations, together with reduced availability of the snRNP components of the U12 spliceosome induced by low SMN levels, can explain the accumulation of SMN-dependent U12 splicing defects seen in the Examples with SMA. mRNA levels of U12 intron-containing genes are likely reduced due to degradation by surveilance mechanisms of incorrectly processed mRNAs resulting from SMN-dependent disruption of U12 splicing.

Embodiments of the invention are described in the Summary of Invention and otherwise above.

Another embodiment is directed to a microchip or microarray comprising a plurality of oligonucleotides that are complementary to a plurality of mRNAs each encoding a specific diagnostic protein, essentially a panel of oligonucleotides to screen for the expression of normal diagnostic proteins in a biological sample. Such a microchip or microarray is useful for diagnosing an MND in a subject having a symptom of MND to look for under expression of key diagnostic proteins.

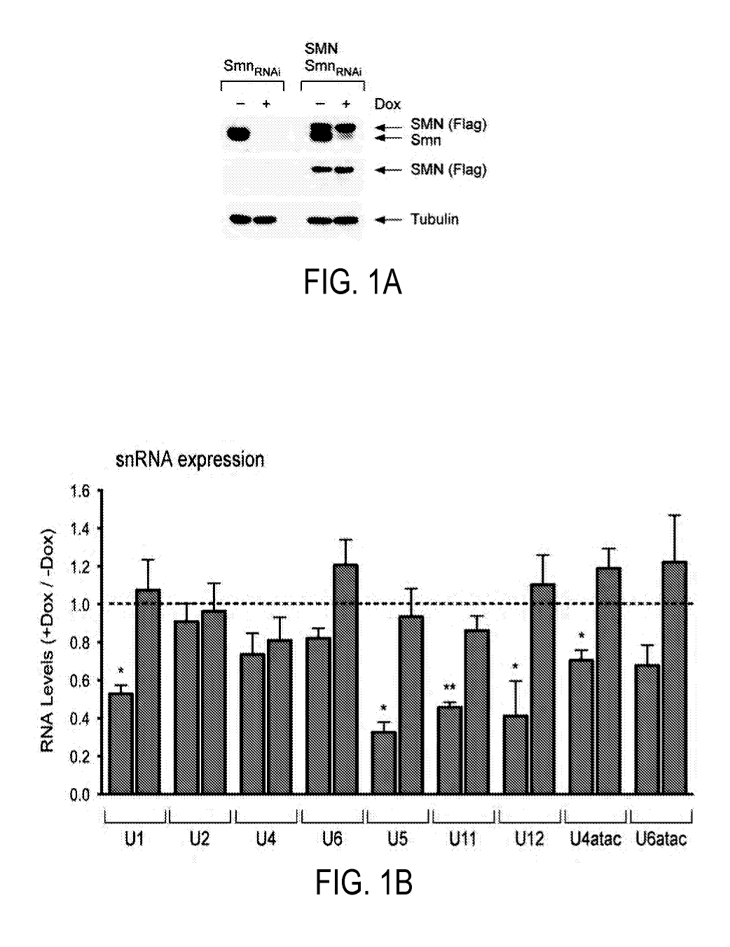

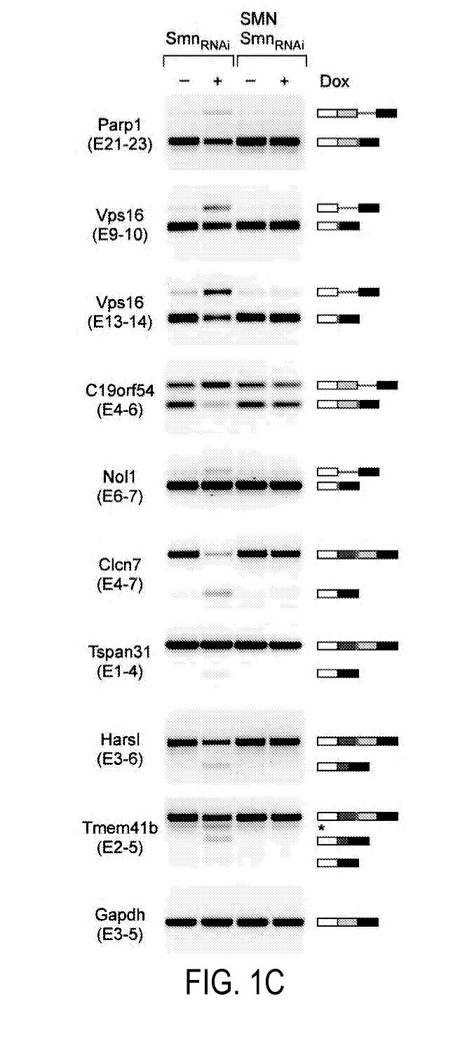

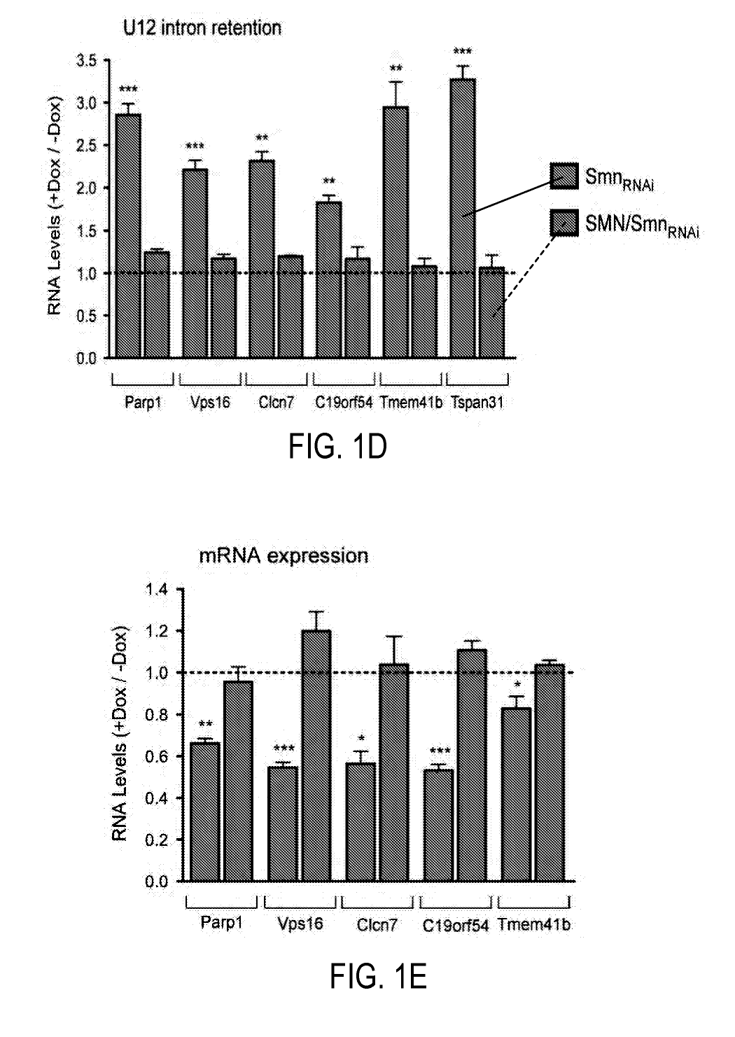

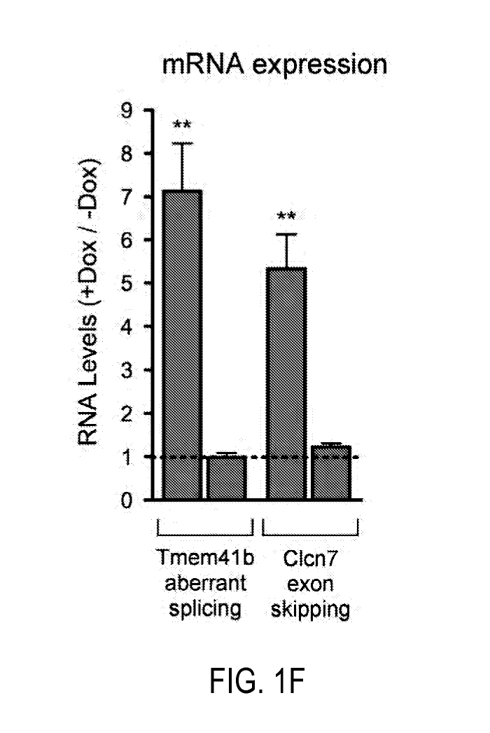

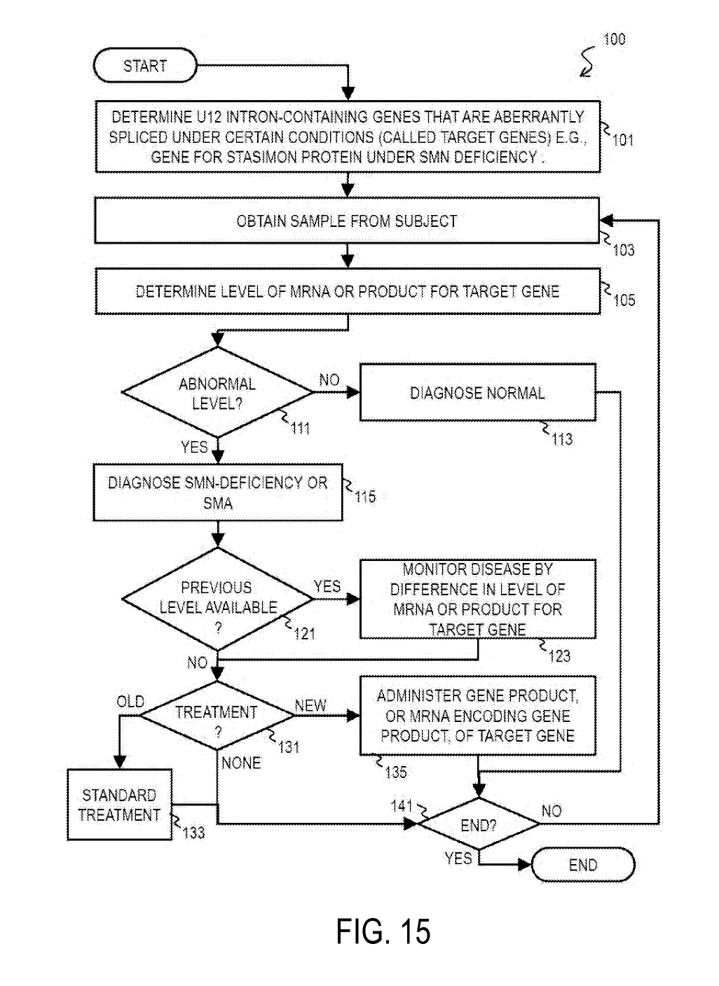

FIG. 15 is a flow chart that illustrates an example method 100 for detecting, monitoring and treating a disease such as SMA, according to an embodiment. Although steps are shown as integral blocks in a particular order in FIG. 1 for purposes of illustration, in other embodiments, one or more steps, or portions thereof, are performed in a different order, or overlapping in time, in series or in parallel, or one or more steps or portions thereof are omitted, or other steps are added, or the method is changed in some combination of ways.

In the flow chart describing various embodiments, in step 101, one or more target diagnostic genes (for the flow chart this is simplified to “target gene”) and/or target diagnostic proteins are identified, which are determined to contain U12 introns that are aberrantly spliced into mRNA under certain conditions, such as under conditions of SMN deficiency. For example, during step 101, it was determined that the Stasimon gene codes for a Stasimon protein that is essential for neuromuscular junction transmission and muscle growth, but is aberrantly spliced under conditions of SMN deficiency that causes SMA.

In step 103, one or more biological samples are collected from a subject, for example blood, skin, muscle, fibroblasts, serum, and cerebrospinal fluid.

In step 105, the level of mRNA for a target gene or of the target diagnostic protein encoded by the gene in the sample is determined, for example using quantitative RT-qPCR. In some embodiments, the level of correctly spliced and aberrantly or alternatively spliced mRNA as well as U12 intron-containing unspliced mRNA transcribed from the target gene are determined. In some embodiments the relative levels of correctly to aberrantly or alternatively spliced mRNA and unspliced mRNA are determined. For example, for the Stasimon gene (in humans it is http://www.ncbi.nlm.nih.gov/nuccore/28278156) and both the normal mRNA and aberrantly or alternatively spliced mRNA sequences as described herein and in the appendices. The amino acid sequence of normal Stasimon protein is identified by http://www.uniprot.org/uniprot/Q5BJD5.

Aberrantly or alternatively spliced Stasimon mRNA and unspliced Stasimon mRNA accumulates in the cell and it is detected and quantified and/or the amount of normal target diagnostic protein encoded by the diagnostic gene is quantified.

In step 111, it is determined whether the level of aberrantly or alternatively spliced Stasimon mRNA and unspliced Stasimon mRNA determined in step 105 is abnormally high in the patient (i.e., is significantly higher than normal levels in a subject known not to have the disease), and/or if the level of normal target diagnostic protein is significantly lower than in normal controls. If the defective mRNA levels are not higher or if the level of the normally spliced mRNA or of the target diagnostic protein is not lower than normal, then control passes to step 113 where it is determined that the subject is normal. No further action is warranted, and control jumps to step 141 to determine whether the process ends, as described below. The methods need to be adapted to take into account inter-individual variations, which are known to be high.

However, if it is determined that the level of aberrantly or alternatively spliced mRNA and unspliced Stasimon mRNA is abnormally high, or that the level of the normally spliced mRNA or of the target diagnostic protein is significantly lower than normal, then, in step 115, the subject is diagnosed with the SMN deficiency or SMA or MND or other disease associated with SMN deficiency.

Step 121 is related to monitoring disease progression or disease treatment or both. If there has been a measurement of a pre-disease or pre-treatment level of aberrantly or alternatively spliced Stasimon mRNA for the subject (or for other subjects of the same class), then disease progression and response to therapy can be monitored in step 123, by comparing the level of aberrantly or alternatively spliced Stasimon mRNA and unspliced Stasimon mRNA measured in step 105 with the predisease or pretreatment level. In the context of monitoring disease progression, if there is little or insignificant difference, the disease is stable. If the level of aberrantly or alternatively spliced Stasimon mRNA and unspliced Stasimon mRNA increases, then the disease is determined to have progressed. If the level of aberrantly spliced mRNA and unspliced Stasimon mRNA decreases, then the disease is determined to have decreased in likelihood or severity. Further in this stepof normally spliced mRNA can be measured, which is expected to behave in an opposite manner to the mis-spliced forms—i.e. lower in SMA relative to controls and increased/restored upon effective treatment. If the subject is being treated for the disease, then a significant increase in level of aberrantly or alternatively spliced Stasimon mRNA and unspliced Stasimon mRNA after the onset of treatment, compared to pretreatment levels, indicates that the treatment is not effective (the degree of ineffectiveness is proportional to the increase in the level of aberrantly spliced mRNA). However, if the level of aberrantly spliced mRNA and unspliced Stasimon mRNA is significantly reduced following treatment, then treatment is determined to be effective.

In step 131 the appropriate treatment decision is determined. If the patient is not responding to an existing treatment, a new treatment is selected in step 131. In step 135 pre- and post-treatment mRNA levels are monitored to determine efficacy of the treatment.

In step 141, it is determined whether the process ends or the subject should provide another sample at a later time. If the subject should provide another sample, then control passes back to step 103 and the sequence begins again.

### Motor Neuron Diseases

MNDs are classified as either inherited or sporadic, and according to whether degeneration affects upper motor neurons, lower motor neurons, or both. For example, primary lateral sclerosis is a disease of the upper motor neurons, while progressive muscular atrophy affects only lower motor neurons in the spinal cord. In progressive bulbar palsy, the lowest motor neurons of the brain stem are most affected, causing slurred speech and difficulty chewing and swallowing. If the MND is inherited, it is also classified according to the mode of inheritance. Autosomal dominant means that a person needs to inherit only one copy of the defective gene from one affected parent to be at risk of the disease. There is a 50 percent chance that each child of an affected person will be affected. Autosomal recessive means the individual must inherit a copy of the defective gene from both parents. SMA is an autosomal recessive disorder caused by defects in the gene SMN1, which makes a protein that is important for the survival of motor neurons (SMN protein).

SMA is an MND involving the lower motor neurons that is caused by homozygous inactivation of the Survival Motor Neuron 1 (SMN1) gene (Lefebvre et al., 1995). This autosomal recessive disease is one of the leading genetic causes of infant deaths. In SMA, insufficient levels of the SMN protein lead to degeneration of the lower motor neurons producing weakness and wasting of the skeletal muscles. This weakness is often more severe in the trunk and upper leg and arm muscles than in muscles of the hands and feet. SMA in children is classified into three types, based on ages of onset, severity, and progression of symptoms. All three types are caused by defects in the SMN1 gene.

SMA type I, also called Werdnig-Hoffmann disease, is evident by the time a child is 6 months old. Symptoms may include hypotonia (severely reduced muscle tone), diminished limb movements, lack of tendon reflexes, fasciculations, tremors, swallowing and feeding difficulties, and impaired breathing. Affected children never sit or stand and the vast majority usually die of respiratory failure before the age of 2.

Symptoms of SMA type II, the intermediate form, usually begin between 6 and 18 months of age. Children may be able to sit but are unable to stand or walk unaided, and may have respiratory difficulties. The progression of disease is variable. Life expectancy is reduced but some individuals live into adolescence or young adulthood.

Symptoms of SMA type III (Kugelberg-Welander disease) appear between 2 and 17 years of age and include abnormal gait; difficulty running, climbing steps, or rising from a chair; and a fine tremor of the fingers. The lower extremities are most often affected. Complications include scoliosis and joint contractures—chronic shortening of muscles or tendons around joints, caused by abnormal muscle tone and weakness, which prevents the joints from moving freely. Individuals with SMA type III may be prone to respiratory infections, but with care may have a normal lifespan.

Congenital SMA with arthrogryposis (persistent contracture of joints with fixed abnormal posture of the limb) is a rare disorder. Manifestations include severe contractures, scoliosis, chest deformity, respiratory problems, unusually small jaws, and drooping of the upper eyelids

A brief description of the symptoms of some common MNDs follows.

ALS, also called Lou Gehrig's disease or classical motor neuron disease, is a progressive, ultimately fatal disorder that involves upper and lower motor neurons and disrupts signals to all voluntary muscles Symptoms are usually noticed first in the arms and hands, legs, or swallowing muscles. Approximately 75 percent of people with classic ALS will develop bilateral weakness and atrophy of the bulbar muscles (muscles that control speech, swallowing, and chewing). Affected individuals lose strength and the ability to move their arms and legs, and to hold the body upright. Other symptoms include spasticity, spasms, muscle cramps, and fasciculations. Speech can become slurred or nasal. When muscles of the diaphragm and chest wall fail to function properly, individuals lose the ability to breathe without mechanical support. Although the disease does not usually impair a person's mind or personality, several recent studies suggest that some people with ALS may develop cognitive problems involving word fluency, decision-making, and memory. Most individuals with ALS die from respiratory failure, usually within 3 to 5 years from the onset of symptoms. However, about 10 percent of affected individuals survive for 10 or more years.

ALS most commonly strikes people between 40 and 60 years of age, but younger and older individuals also can develop the disease. Men are affected more often than women. Most cases of ALS occur sporadically, and family members of those individuals are not considered to be at increased risk for developing the disease. Familial forms of ALS account for 10 percent or less of cases of ALS, with more than 10 genes identified to date. However, most of the gene mutations discovered account for a very small number of cases. The most common familial forms of ALS in adults are caused by mutations of the superoxide dismutase gene, or SOD1, located on chromosome 21. There are also rare juvenile-onset forms of familial ALS.

Progressive bulbar palsy, also called progressive bulbar atrophy, involves the brain stem—the bulb-shaped region containing lower motor neurons needed for swallowing, speaking, chewing, and other functions. Symptoms include pharyngeal muscle weakness (involved with swallowing), weak jaw and facial muscles, progressive loss of speech, and tongue muscle atrophy. Limb weakness with both lower and upper motor neuron signs is almost always evident but less prominent. Individuals are at increased risk of choking and aspiration pneumonia, which is caused by the passage of liquids and food through the vocal folds and into the lower airways and lungs. Affected persons have outbursts of laughing or crying (called emotional lability). Stroke and myasthenia gravis may have certain symptoms that are similar to those of progressive bulbar palsy and must be ruled out prior to diagnosing this disorder. In about 25 percent of individuals with ALS, early symptoms begin with bulbar involvement. Some 75 percent of individuals with classic ALS eventually show some bulbar involvement. Many clinicians believe that progressive bulbar palsy by itself, without evidence of abnormalities in the arms or legs, is extremely rare.

Pseudobulbar palsy, which shares many symptoms of progressive bulbar palsy, is characterized by degeneration of upper motor neurons that transmit signals to the lower motor neurons in the brain stem. Affected individuals have progressive loss of the ability to speak, chew, and swallow. Progressive weakness in facial muscles leads to an expressionless face. Individuals may develop a gravelly voice and an increased gag reflex. The tongue may become immobile and unable to protrude from the mouth. Individuals may have outbursts of laughing or crying.

Primary lateral sclerosis (PLS) affects the upper motor neurons of the arms, legs, and face. It occurs when specific nerve cells in the motor regions of the cerebral cortex (the thin layer of cells covering the brain which is responsible for most high-level brain functions) gradually degenerate, causing the movements to be slow and effortful. The disorder often affects the legs first, followed by the body trunk, arms and hands, and, finally, the bulbar muscles. Speech may become slowed and slurred. When affected, the legs and arms become stiff, clumsy, slow and weak, leading to an inability to walk or carry out tasks requiring fine hand coordination. Difficulty with balance may lead to falls. Speech may become slow and slurred. Affected individuals commonly experience pseudobulbar affect and an overactive startle response. PLS is more common in men than in women, with a very gradual onset that generally occurs between ages 40 and 60. The cause is unknown. The symptoms progress gradually over years, leading to progressive stiffness and clumsiness of the affected muscles. PLS is sometimes considered a variant of ALS, but the major difference is the sparing of lower motor neurons, the slow rate of disease progression, and normal lifespan. PLS may be mistaken for spastic paraplegia, a hereditary disorder of the upper motor neurons that causes spasticity in the legs and usually starts in adolescence. Most neurologists follow the affected individual's clinical course for at least 3 to 4 years before making a diagnosis of PLS. The disorder is not fatal but may affect quality of life.

Progressive muscular atrophy is marked by slow but progressive degeneration of only the lower motor neurons. It largely affects men, with onset earlier than in other MNDs. Weakness is typically seen first in the hands and then spreads into the lower body, where it can be severe. Other symptoms may include muscle wasting, clumsy hand movements, fasciculations, and muscle cramps. The trunk muscles and respiration may become affected. Exposure to cold can worsen symptoms. The disease develops into ALS in many instances.

Kennedy's disease, also known as progressive spinobulbar muscular atrophy, is an X-linked recessive disease caused by mutations in the gene for the androgen receptor.

There are no specific tests to diagnose most MNDs although there are now gene tests for SMA. Symptoms may vary among individuals and, in the early stages of the disease, may be similar to those of other diseases, making diagnosis difficult. There is no cure or standard treatment for the MNDs. Symptomatic and supportive treatment can help people be more comfortable while maintaining their quality of life. Multidisciplinary clinics, with specialists from neurology, physical therapy, respiratory therapy, and social work are particularly important in the care of individuals with MNDs. At present, the drug riluzole (Rilutek®), the only prescribed drug approved by the U.S. Food and Drug Administration to treat ALS, prolongs life by 2-3 months but does not relieve symptoms. The drug reduces the body's natural production of the neurotransmitter glutamate, which carries signals to the motor neurons.

### Gene Therapy

Gene therapy of mice in vivo with AAV-Stasimon provides phenotypic benefit by increasing motor activity, weight gain and survival in the SMNA7 mouse model of severe SMA. Methods are well known in the art for making AAV carrying a protein of interest. For example, Kaspar, Brian K., et al. 20120177605 Delivery of Polynucleotides Across the Blood-Brain-Barrier Using Recombinant AAV9.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple serotypes of AAV exist and offer varied tissue tropism. Known serotypes include, for example, AAV1, AAV2, AAV3, AAV4, AAV5, AAV6, AAV7, AAV8, AAV9, AAV10 and AAV11. AAV9 is described in U.S. Pat. No. 7,198,951 and in Gao, et al., J. Virol., 78: 6381-6388 (2004). Advances in the delivery of AAV6 and AAV8 have made possible the transduction by these serotypes of skeletal and cardiac muscle following simple systemic intravenous or intraperitoneal injections. See Pacak et al., Circ. Res., 99 (4): 3-9 (1006) and Wang et al., Nature Biotech., 23 (3): 321-8 (2005).

In those methods of the invention for systemically delivering polynucleotides to the spinal cord, use of the methods and materials is indicated, for example, for lower motor neuron diseases such as SMA and ALS and others described herein.

Recombinant AAV (rAAV) is stored in a pharmaceutically acceptable carrier. The AAV composition may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1×10⁶, to about 1×10¹⁴ or more DNase resistant particles (DRP) per ml. Dosages may also be expressed in units of viral genomes (vg). Dosages may also vary based on the timing of the administration to a human. These dosages of rAAV may range from about 1×10¹¹ vg/kg, to about 1×10¹⁶ or more viral genomes per kilogram body weight in an adult. For a neonate, the dosages of rAAV may range from about 1×10¹¹, to about 3×10¹⁶ or more viral genomes per kilogram body weight.

Methods of transducing nerve or glial target cells with rAAV carrying the diagnostic proteins, such as Stasimon or Rashomon or both, are contemplated by the invention. The methods comprise the step of administering an intravenous effective dose, or effective multiple doses, of a composition comprising a rAAV carrying the diagnostic protein(s) to an animal (including a human being) diagnosed with an MND, in particular SMA, and in need thereof. If the dose is administered after the development of a disorder/disease, the administration is therapeutic. In embodiments of the invention, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Examples of disease states contemplated for treatment by methods of the invention are listed herein above.

Combination therapies are also contemplated by the invention. Combination as used herein includes both simultaneous treatments or sequential treatments with the AAV-diagnostic protein(s) and convention therapy. For example, combinations of methods of the invention with standard medical treatments (e.g., riluzole in ALS) are specifically contemplated, as are combinations with novel therapies.

Route(s) of administration and serotype(s) of AAV may be chosen and/or matched by those skilled in the art taking into account the disease state being treated. While delivery to an individual in need thereof after birth is contemplated, intrauteral delivery and delivery to the mother are also contemplated.

Compositions suitable for systemic use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of adispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin, and Tween family of products (e.g., Tween 20).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction of cells with rAAV of the invention results in sustained expression of diagnostic protein(s). The present invention thus provides methods of administering/delivering rAAV (e.g., encoding as Stasimon or Rashomon or both or other combination of one or more diagnostic proteins) to an animal or a human patient. An embodiment is directed to a rAAV comprising stasimon, Rashomon, or both or other combination of one or more diagnostic proteins, preferably an AAV comprising a self-complementary genome, These methods include transducing nerve and/or glial cells with one or more rAAV of the present invention. Transduction may be carried out with gene cassettes comprising tissue specific control elements. For example, promoters that allow expression specifically within neurons or specifically within other cell types such as but not limited to astrocytes. Examples include neuron specific enolase and glial fibrillary acidic protein promoters. Inducible promoters under the control of an ingested drug may also be developed.

Other types of vectors or gene delivery systems are known in the art, including adenovirus and lentivirus.

### Protein Variants

Variants of SMN or Stasimon or other diagnostic proteins discussed herein, and biologically active fragments thereof, include forms that are substantially homologous but derived from another organism, i.e., an ortholog. Variants also include proteins or peptides that are substantially homologous that are produced by chemical synthesis or by recombinant methods.

As used herein, two proteins (or a region of the proteins or peptides) are substantially homologous when the amino acid sequences are at least about 70-75%, typically at least about 80-85%, and most typically having at least about 90-95%, 97%, 98% or 99% or more sequence identity. Variants include conservative amino acid substitutions such as the substitution of amino acids whose side chains have similar biochemical properties).

A variant polypeptide can differ in amino acid sequence by one or more substitutions, deletions, insertions, inversions, fusions, or truncations or a combination of any of these. Variant polypeptides can be fully functional or can lack function in one or more activities.

Fully functional variants typically contain only conservative variation or variation in non-critical residues or in non-critical regions. Functional variants can also contain substitution of similar amino acids, which results in no change or an insignificant change in function.

As indicated, variants can be naturally occurring or can be made by recombinant means of chemical synthesis to provide useful and novel characteristics of the desired protein.

Substantial homology can be to the entire nucleic acid or amino acid sequence or to fragments of these sequences. Fragments can be derived from the full naturally occurring amino acid sequence. However, the invention also encompasses fragments of the variants as described herein. Accordingly, a fragment can comprise any length that retains one or more of the desired biological activities of the protein. Fragments can be discrete (not fused to other amino acids or polypeptides) or can be within a larger polypeptide. Further, several fragments can be comprised within a single larger polypeptide.

Polypeptides often contain amino acids other than the 20 amino acids commonly referred to as the 20 naturally occurring amino acids. Further, many amino acids, including the terminal amino acids, may be modified by natural processes, such as processing and other post-translational modifications, or by chemical modification techniques well known in the art. Common modifications that occur naturally in polypeptides are described below.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

### Spliceosome

A “spliceosome,” as used herein, means a large macromolecular RNA-protein complex formed as a result of snRNPs involved in splicing together with many additional proteins. This complex of snRNA and protein subunits removes introns from a transcribed pre-mRNA (hnRNA) segment. Such a process is generally referred to as splicing. Each spliceosome is composed of five small nuclear RNAs (snRNA), and a range of associated protein factors. When these small RNAs are combined with the protein factors, they make an RNA-protein complex called snRNP. The snRNAs that make up the nuclear spliceosome are named U1, U2, U4, U5, and U6, and participate in several RNA-RNA and RNA-protein interactions. The RNA component of the small nuclear ribonucleic protein or snRNP (pronounced “snurp”) is rich in uridine (the nucleoside analog of the uracil nucleotide).

snRNP

“snRNPs” (pronounced “snurps”), or small nuclear ribonucleoproteins, as used herein mean RNA-protein complexes that combine with unmodified pre-mRNA and various other proteins to form a spliceo some, a large RNA-protein molecular complex upon which splicing of pre-mRNA occurs. The action of snRNPs is essential to the removal of introns from pre-mRNA, a critical aspect of post-transcriptional modification of RNA, occurring only in the nucleus of eukaryotic cells. The two essential components of snRNPs are protein molecules and RNA. The RNA found within each snRNP particle is known as small nuclear RNA, or snRNA, and is usually about 150 nucleotides in length. The snRNA component of the snRNP gives specificity to individual introns by “recognizing” the sequences of critical splicing signals at the 5′ and 3′ ends and branch site of introns. The snRNA in snRNPs is similar to ribosomal RNA in that it directly incorporates both an enzymatic and a structural role.

U12 Introns and 5′UTRs are defined above.

### Nucleic Acids

“Nucleic acid” shall mean any nucleic acid molecule, including, without limitation, DNA, RNA and hybrids (chimeras) thereof. The term “nucleic acid” can also refer to a deoxyribonucleotide or ribonucleotide, whether singular or in polymers, naturally occurring or non-naturally occurring, double-stranded or single-stranded, coding (e.g. translated gene) or non-coding (e.g. regulatory region), or any fragments, derivatives, mimetics or complements thereof. The nucleic acid bases that form nucleic acid molecules can be the bases A, C, G, T and U, as well as derivatives or modifications thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

Examples of nucleic acids include oligonucleotides, nucleotides, polynucleotides, nucleic acid sequences, genomic sequences, antisense nucleic acids, DNA regions, probes, primers, genes, regulatory regions, introns, and exons, open-reading frames, binding sites, target nucleic acids and allele-specific nucleic acids. A nucleic acid can include one or more polymorphisms, variations or mutations (e.g., SNPs, insertions, deletions, inversions, translocations, etc.). A nucleic acid includes analogs (e.g., phosphorothioates, phosphoramidates, methyl phosphonate, chiral-methyl phosphonates, 2-O-methyl ribonucleotides) or modified nucleic acids (e.g., modified backbone residues or linkages) or nucleic acids that are combined with carbohydrates, lipids, protein or other materials, or peptide nucleic acids (PNAs) (e.g., chromatin, ribosomes, transcriptosomes, etc.) or nucleic acids in various structures (e.g., A DNA, B DNA, Z-form DNA, siRNA, tRNA, ribozymes, etc.).

Nucleic acids in the context of this invention include “oligonucleotides,” which refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. DNA/RNA chimeras are also included.

Nucleic acids for use in embodiments of the present invention may be of various lengths, generally dependent upon the particular form of nucleic acid, typically from about 10 to 100 nucleotides in length. In various related embodiments, oligonucleotides, single-stranded, double-stranded, and triple-stranded, may range in length from about 10 to about 50 nucleotides, from about 20 o about 50 nucleotides, from about 15 to about 30 nucleotides, from about 20 to about 30 nucleotides in length.

### Hybridization

In the context of this invention, “hybridization” means hydrogen bonding, which may be Watson-Crick, Hoogsteen or reversed Hoogsteen hydrogen bonding, between complementary nucleoside or nucleotide bases. “Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently. For example, adenine and thymine are complementary nucleobases which pair through the formation of hydrogen bonds.

“Complementary,” as used herein, refers to the capacity for precise pairing between two nucleotides. For example, if a nucleotide at a certain position of a nucleic acid is capable of hydrogen bonding with a nucleotide at the same position of a DNA or RNA molecule, then the nucleic acid and the DNA or RNA are considered to be complementary to each other at that position. The nucleic acid and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the nucleic acid and the DNA or RNA target.

Various conditions of stringency can be used for hybridization as is described below. Hybridization can be carried out under conditions varying in stringency, preferably under conditions of high stringency, e.g., 6× SSPE, at 65 degrees Celsius, to allow for hybridization of complementary sequences having extensive homology, usually having no more than one or two mismatches in a probe of 25 nucleotides in length, i.e., at least 95% to 100% sequence identity. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1 6.3.6, which is incorporated by reference. Aqueous and nonaqueous methods are described in that reference and either can be used. Specific hybridization conditions referred to herein are as follows: 1) low stringency hybridization conditions in 6×sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

### PCR

Target nucleic acids are amplified to obtain amplification products. Suitable nucleic acid amplification techniques are well known to a person of ordinary skill in the art, and include polymerase chain reaction (PCR) as for example described in Ausubel et al., Current Protocols in Molecular Biology (John Wiley & Sons, Inc. 1994-1998) (and incorporated herein). The most commonly used nucleic acid amplification technique is the polymerase chain reaction (PCR). PCR is well known in this field and comprehensive description of this type of reaction is provided in E. van Pelt-Verkuil et al., Principles and Technical Aspects of PCR Amplification, Springer, 2008. PCR is a powerful technique that amplifies a target DNA sequence against a background of complex DNA. If RNA is to be amplified (by PCR), it must be first transcribed into cDNA (complementary DNA) using an enzyme called reverse transcriptase. Afterwards, the resulting cDNA is amplified by PCR. PCR is an exponential process that proceeds as long as the conditions for sustaining the reaction are acceptable. The components of the reaction are: i. pair of primers—short single strands of DNA with around 10-30 nucleotides complementary to the regions flanking the target sequence; ii. DNA polymerase—a thermostable enzyme that synthesizes DNA; iii. deoxyribonucleoside triphosphates (dNTPs)—to provide the nucleotides that are incorporated into the newly synthesized DNA strand; and [0027] iv. buffer—to provide the optimal chemical environment for DNA synthesis.

PCR typically involves placing these reactants in a small tube (˜10-50 μl) containing the extracted nucleic acids. The tube is placed in a thermal cycler; an instrument that subjects the reaction to a series of different temperatures for varying amounts of time. The standard protocol for each thermal cycle involves a denaturation phase, an annealing phase, and an extension phase. The extension phase is sometimes referred to as the primer extension phase. In addition to such three-step protocols, two-step thermal protocols can be employed, in which the annealing and extension phases are combined. The denaturation phase typically involves raising the temperature of the reaction to 90-95° C. to denature the DNA strands; in the annealing phase, the temperature is lowered to ˜50-60° C. for the primers to anneal; and then in the extension phase the temperature is raised to the optimal DNA polymerase activity temperature of 60-72° C. for primer extension. This process is repeated cyclically around 20-40 times.

There are a number of variants to the standard PCR protocol such as multiplex PCR, linker-primed PCR, direct PCR, tandem PCR, real-time PCR and reverse-transcriptase PCR, amongst others, which have been developed for molecular diagnostics.

Multiplex PCR uses multiple primer sets within a single PCR mixture to produce amplicons of varying sizes that are specific to different DNA sequences. By targeting multiple genes at once, additional information may be gained from a single test-run that otherwise would require several experiments. Optimization of multiplex PCR is more difficult though and requires selecting primers with similar annealing temperatures, and amplicons with similar lengths and base composition to ensure the amplification efficiency of each amplicon is equivalent.

Linker-primed PCR, also known as ligation adaptor PCR, is a method used to enable nucleic acid amplification of essentially all DNA sequences in a complex DNA mixture without the need for target-specific primers. The method firstly involves digesting the target DNA population with a suitable restriction endonuclease (enzyme). Double-stranded oligonucleotide linkers (also called adaptors) with a suitable overhanging end are then ligated to the ends of target DNA fragments using a ligase enzyme. Nucleic acid amplification is subsequently performed using oligonucleotide primers which are specific for the linker sequences. In this way, all fragments of the DNA source which are flanked by linker oligonucleotides can be amplified.

Direct PCR describes a system whereby PCR is performed directly on a sample without any, or with minimal, nucleic acid extraction. It has long been accepted that PCR reactions are inhibited by the presence of many components of unpurified biological samples, such as the heme component in blood. Traditionally, PCR has required extensive purification of the target nucleic acid prior to preparation of the reaction mixture. With appropriate changes to the chemistry and sample concentration, however, it is possible to perform PCR with minimal DNA purification, or direct PCR. Adjustments to the PCR chemistry for direct PCR include increased buffer strength, the use of polymerases which have high activity and processivity, and additives

Tandem PCR utilizes two distinct rounds of nucleic acid amplification to increase the probability that the correct amplicon is amplified. One form of tandem PCR is nested PCR in which two pairs of PCR primers are used to amplify a single locus in separate rounds of nucleic acid amplification. The first pair of primers hybridize to the nucleic acid sequence at regions external to the target nucleic acid sequence. The second pair of primers (nested primers) used in the second round of amplification bind within the first PCR product and produce a second PCR product containing the target nucleic acid, that will be shorter than the first one. The logic behind this strategy is that if the wrong locus were amplified by mistake during the first round of nucleic acid amplification, the probability is very low that it would also be amplified a second time by a second pair of primers and thus ensures specificity.

Real-time PCR, or quantitative PCR, is used to measure the quantity of a PCR product in real time. By using a fluorophore-containing probe or fluorescent dyes along with a set of standards in the reaction, it is possible to quantitate the starting amount of nucleic acid in the sample. This is particularly useful in molecular diagnostics where treatment options may differ depending on the pathogen load in the sample.

### RT-PCR

Typically DNA sequences are amplified, although in some instances RNA sequences can be amplified or converted into cDNA, such as by using RT PCR. Reverse-transcriptase PCR (RT-PCR) is used to amplify DNA from RNA. Reverse transcriptase is an enzyme that reverse transcribes RNA into complementary DNA (cDNA), which is then amplified by PCR. RT-PCR is widely used in expression profiling, to determine the expression of a gene or to identify the sequence of an RNA transcript, including transcription start and termination sites. It is also used to amplify RNA viruses such as human immunodeficiency virus or hepatitis C virus. “cDNA” or “complementary DNA” is DNA synthesized from a messenger RNA (mRNA) template in a reaction catalyzed by the enzyme reverse transcriptase and the enzyme DNA polymerase. Complementary base sequences are those sequences that are related by the base-pairing rules. In DNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

A reverse transcriptase PCRTM amplification procedure may be performed when the source of nucleic acid is fractionated or whole cell RNA. Methods of reverse transcribing RNA into cDNA are well known (see Sambrook et al., 1989). Alternative methods for reverse polymerization utilize thermostable DNA polymerases. These methods are described in WO 90/07641. Representative methods of RT-PCR are described in U.S. Pat. No. 5,882,864.

### Pharmaceutical Compositions or Formulations

The diagnostic proteins of the present invention can be formulated in various ways including as or salts or derivatives thereof, for storage or administration depending on the particular need of the subject and the bone disease being treated. The diagnostic proteins were chemically synthesized (95% pure) and therefore have residue Trifluoroacetic acid (TFA). Almost all peptide drugs are in the TFA salt form. Typically peptide drugs for clinical trials are in the acetate salt form. For the experiments described herein, lyophilized peptides in 0.9% NaCl+100 mM Acetic acid were stored at −20° C. until they were used. Pharmaceutical compositions comprising the dp, can be formulated in any suitable amount, vehicle or pharmaceutically acceptable carrier for delivery. It is within the invention to provide a pharmaceutical composition, wherein the dp, are present in an amount effective to treat MND.

The pharmaceutical compositions of the present invention may be formulated and used as tablets, capsules, or elixirs for oral or buccal administration; for use in vaginal or rectal administration particularly in semisolid forms such as creams and suppositories. They may also be formulated in sterile solutions and suspensions for injection, inhalation or pulmonary administration in the form of powders, nasal drops or aerosols.

For therapeutic use, the dp are formulated with a carrier that is pharmaceutically acceptable and is appropriate for delivery by the chosen route of administration. Suitable pharmaceutically acceptable carriers are those used conventionally with peptide-based drugs, such as diluents, excipients and the like. Pharmaceutically acceptable carriers for therapeutic use are well known in the pharmaceutical art, and are described, for example, in Remington's Pharmaceutical Sciences, Mack Publishing Company, Easton, Pa., 19th Edition, 1995).

Pharmaceutical compositions suitable for parenteral administration conveniently comprise a sterile aqueous preparations of the enumerated fragments that are preferably isotonic with the blood of the recipient. This aqueous preparation may be formulated according to known methods using those suitable dispersing or wetting agents and suspending agents An example of an aqueous formulation is a solution in 1,3-butane diol. Water, Ringer's solution, and isotonic sodium chloride solution are exemplary acceptable diluents. Sterile, fixed oils may be employed as a solvent or suspending medium. Bland fixed oils, including synthetic mono or diglycerides, and fatty acids, such as oleic acid, may also be used.

Formulations for vaginal or rectal administration, e.g. suppositories, may contain as excipients, for example, polyalkylene glycols, vaseline, cocoa butter, and the like. Formulations for inhalation administration may be solid and contain as excipients, for example, lactose or may be aqueous or oily solutions for administration in the form of nasal drops. For buccal administration excipients include sugars, calcium stearate, magnesium stearate, pregelinatined starch, and the like.

Preparations of the invention may optionally comprise pharmaceutically acceptable salts, buffering agents, preservatives and excipients. Examples of these salts are those of mineral or organic acids, e.g. of hydrochloric, acetic or methanesulfonic acid. Also salts as alkaline metal or alkaline earth salts, such as sodium or magnesium salts of the carboxylic acid group, are conceivable.

The present dp may be administered in a vehicle, such as distilled water or in saline, phosphate buffered saline, 5% dextrose solutions or oils. The solubility of the dp may be enhanced, if desired, by incorporating a solubility enhancer, such as detergents and emulsifiers.

The dp may be utilized in the form of a sterile-filled vial or ampoule containing a therapeutically effective amount, in either unit dose or multi-dose amounts. The vial or ampoule may contain the dp and the desired carrier, as an administration ready formulation. Alternatively, the vial or ampoule may contain the dp in a form, such as a lyophilized form, suitable for reconstitution in a suitable carrier, such as sterile water or phosphate-buffered saline.

Suitable buffering agents are systems of acetic acid (1-2% w/v), citric acid (1-3% w/v); boric acid (0.5-2.5% w/v), and phosphoric acid (0.8-2% w/v).

Suitable preservatives include benzalkonium chloride (0.003-0.03% w/v); chlorobutanol (0.3-0.9% w/v); parabens (0.01-0.25% w/v) and thimerosal (0.004-0.02% w/v).

In another embodiment of the invention, the pharmaceutical compositions are formulated for administration by infusion, parenteral administration, or by injection, for example subcutaneous, intraperitoneal or intravenous injection, and are accordingly utilized as aqueous solutions in sterile and pyrogen-free form and optionally buffered to physiologically tolerable pH. Formulation for intramuscular administration may be based on solutions or suspensions in plant oil, e.g. canola oil, corn oil or soy bean oil. These oil based formulations may be stabilized by antioxidants e.g. BHA (butylated hydroxianisole) and BHT (butylated hydroxytoluene).

Various approaches for formulating compositions for use in accordance with this invention are described in the HANDBOOK OF PHARMACEUTICAL EXCIPIENTS, 3^{rd}ed., American Pharmaceutical Association, USA and Pharmaceutical Press UK (2000), and pharmaceutics—the science of dosage form design, Churchill Livingstone (1988).

Pharmaceutical compositions of the present invention may be placed within containers, or kits, along with packaging material which provides instructions regarding the use of such pharmaceutical compositions. Generally such instructions will include a tangible expression describing the reagent concentration, as well as within certain embodiments, relative amounts of excipient ingredients or diluents (e.g., water, saline, or PBS) which may be necessary to reconstitute the pharmaceutical composition.

### Nucleic Acid Arrays

Certain embodiments are directed to a microarrays for detecting one or more of the diagnostic genes or correctly spliced mRNA transcribed from them, the “target genes/mRNA” with respect to microarrays. The array contains oligonucleotide probes (herein “probes”) sufficiently complementary to the identified herein to specifically hybridize with the targeted DNA or mRNA.

It will be readily apparent to one skilled in the art that the exact formulation of probes on an array is not critical as long as the user is able to select probes for inclusion on the array that fulfill the function of selectively hybridizing to the target genes/mRNA. The array can be modified to suit the needs of the user. Thus, analysis of the array can provide the user with information regarding the number and/or presence target genes/mRNA in a given sample. The hybridization of a probe complementary to target genes/mRNA in an array can indicate that the subject from whom the sample was derived is has an MND, such as SMA.

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In one example, the array is formed on a polymer medium, which is a thread, membrane or film. An example of an organic polymer medium is a polypropylene sheet having a thickness on the order of about 1 mm (0.001 inch) to about 20 mm although the thickness of the film is not critical and can be varied over a fairly broad range. Biaxially oriented polypropylene (BOPP) films are also suitable in this regard; in addition to their durability, BOPP films exhibit a low background fluorescence. In a particular example, the array is a solid phase, Allele-Specific Oligonucleotides (ASO) based nucleic acid array.

The array formats of the present disclosure can be included in a variety of different types of formats. A “format” includes any format to which the solid support can be affixed, such as microtiter plates, test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (see PCT Publication No. WO 85/01051 and PCT Publication No. WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90 degrees to permit synthesis to proceed within a second (2degrees) set of rows that are now perpendicular to the first set. This process creates a multiple-channel array whose intersection generates a plurality of discrete cells. In particular examples, the oligonucleotide probes on the array include one or more labels, which permit detection of oligonucleotide probe: target sequence hybridization complexes.

### Kits

Certain embodiments are directed to kits. The disclosed kits include a binding molecule, such as an oligonucleotide probe that selectively hybridizes the particular known target genes/mRNA. Alternatively or additionally, the kits can include one or more isolated primers or primer pairs for amplifying the target genes/mRNA.

The kit can further include one or more of a buffer solution, a conjugating solution for developing the signal of interest, or a detection reagent for detecting the signal of interest, each in separate packaging, such as a container. In another example, the kit includes a plurality of size-associated marker of target nucleic acid sequences for hybridization with a detection array. The kit can also include instructions in a tangible form, such as written instructions or in a computer-readable format.

Kits comprising a primer or probe that is complementary to and specifically hybridizes to or binds to a target genes/mRNA in a sample and enzymes suitable for amplifying target genes/mRNA are provided in certain embodiments of the invention. The primer or probe may be labeled with a radioisotope, a fluorophore, a chromophore, a dye, an enzyme, or TOF carrier. In these kits, binding may be detected by in situ hybridization, PCR RT-PCR, fluorescence resonance energy transfer, chemiluminescence enzymatic signal amplification, electron dense particles magnetic particles and capacitance coupling. The probe is selected to allow the target genes/mRNA to be sequenced if wanted, or for quantitation of the respective different target genes/mRNA as compared to the wild-type sequence. These reagents in certain embodiments may comprise one or more nucleic acid probes, may be in the form of a microarray, are suitable for primer extension and can comprise controls indicative of a healthy individual.

### EXAMPLES

### Example 1

### Materials and Methods

### NIH3T3 Cell Lines and Tissue Culture

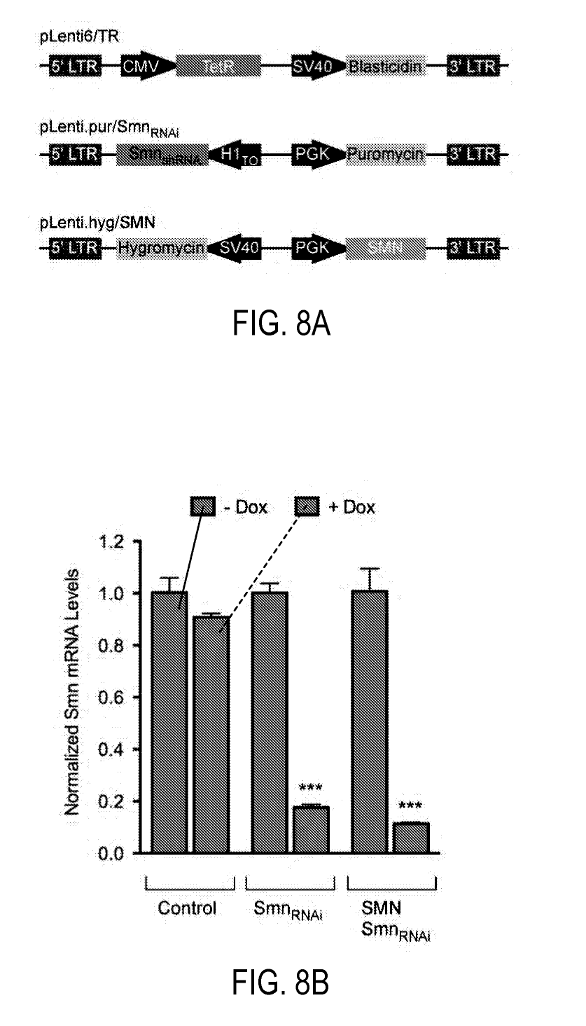

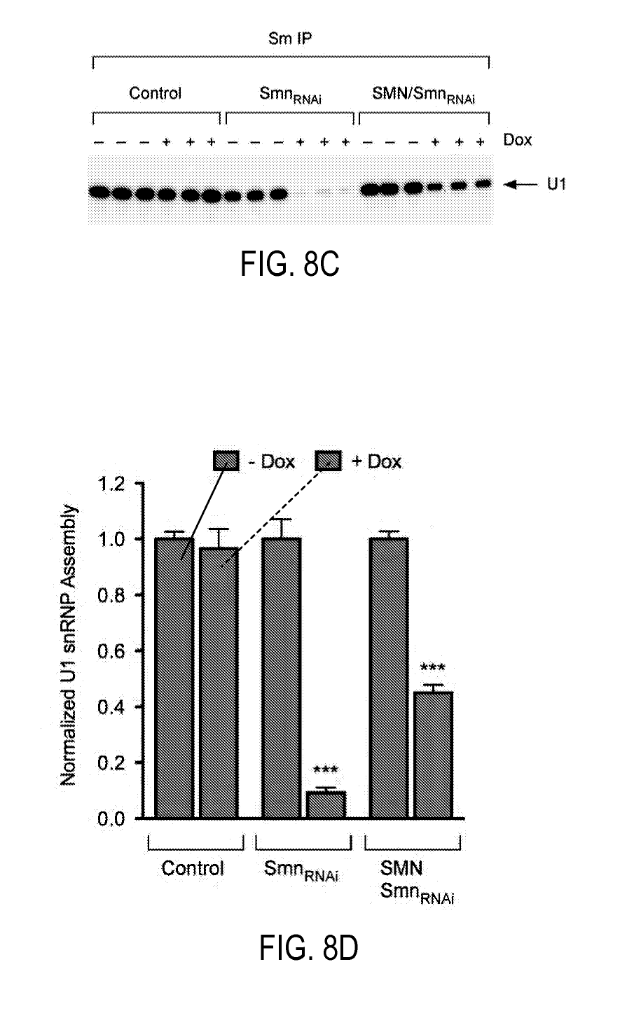

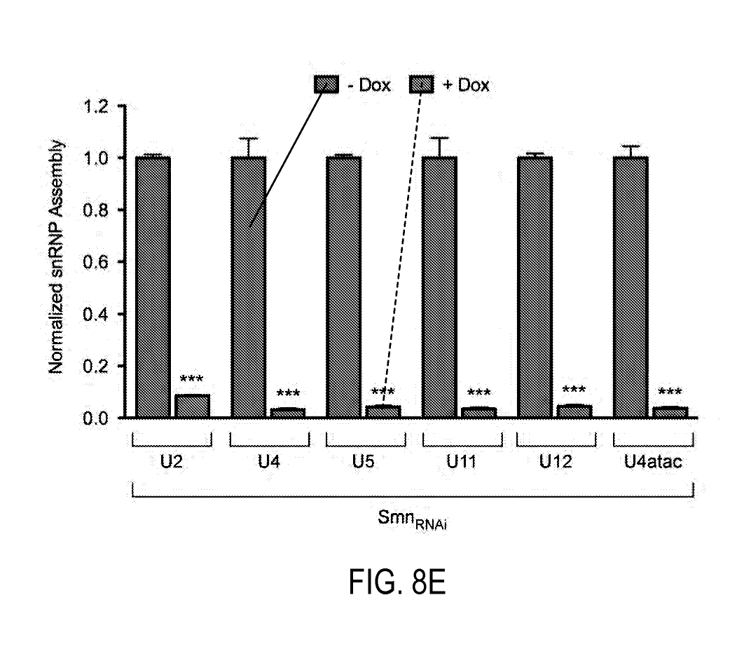

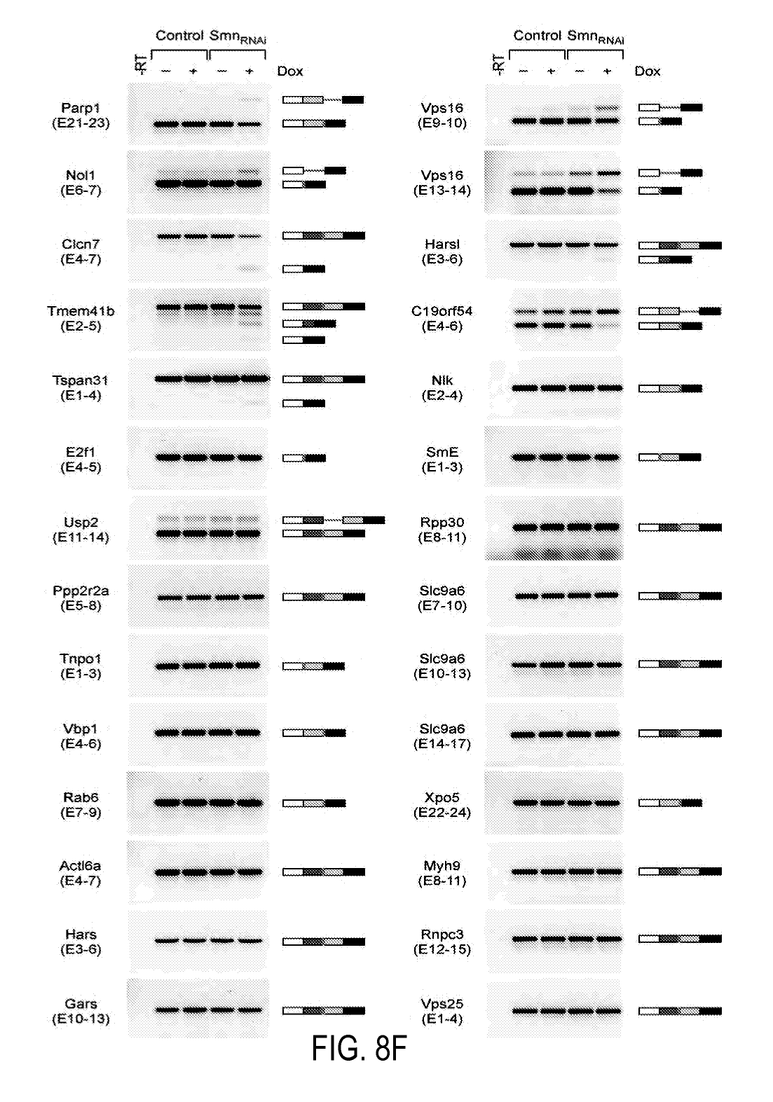

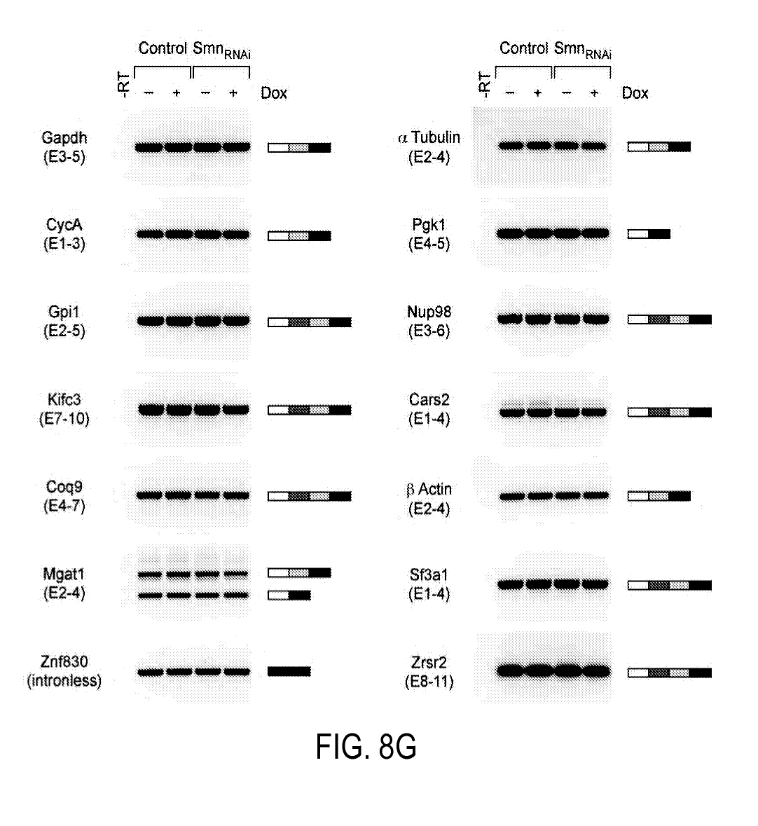

The NIH3T3 cell lines used in this study were generated through lentiviral transduction. 48 hours after lentiviral transduction carried out as described previously (Dull et al., 1998), NIH3T3 cells were split at a 1 to 5 ratio and grown in medium containing the appropriate antibiotic at the following final concentrations: 5 μg/ml Blasticidin-S hydrochloride (Invitrogen), 5 μg/ml Puromycin (Sigma), and 250 μg/ml Hygromycin B (Invitrogen). NIH3T3-Smn_{RNAi} cells were generated by transduction of wild-type NIH3T3 cells with pLenti6/TR and pLenti.pur/Smn_{RNAi} followed by antibiotic selection and cloning by limiting dilution. In these cells, TetR binding to the H1_{TO} promoter represses shRNA transcription in normal conditions while addition of the tetracycline analogue doxycycline to the culture medium triggers shRNA expression and RNAi-mediated knockdown of endogenous mouse SMN (FIGS. 1A and 8B). To control for potentially non-specific effects of shRNA expression, NIH3T3-SMN/Smn_{RNAi} cells were generated by transduction of NIH3T3-Smn_{RNAi} cells with pLenti.hyg/SMN followed by antibiotic selection. These cells express an epitope-tagged human SMN isoform that is resistant to RNAi (FIGS. 1A and 8B) and expected to correct defects caused specifically by depletion of endogenous mouse SMN in NIH3T3-Smn_{RNAi} cells.

Mouse NIH3T3 fibroblasts were grown in DMEM with high glucose (Invitrogen) containing 10% of FBS (HyClone), 2 mM glutamine (Gibco), and 1% penicillin and streptmomycin (Gibco). RNAi was induced by addition to the growth medium of Doxycycline HCl (Fisher Scientific) at the final concentration of 100 ng/ml. For serum deprivation experiments, wild-type NIH3T3 cells were cultured in the presence of 2% FBS. Cell number was determined with an automatic digital cell counter (ADAM, Digital Bio).

### Drosophila Genetics and Phenotypes and Analysis

Drosophila smn^{73Ao} (Chan et al., 2003), smn^{X7} (Chang et al., 2008), and U6atac (Otake et al., 2002) mutants were used and additional stocks described below. Drosophila NMJ electrophysiology was performed as previously described (Imlach and McCabe, 2009). Rhythmic motor activity was recorded from muscle 6 of abdominal segment A1 using a semi-intact preparation where the brain and peripheral nerves remained intact. For analysis of locomotion, 60-second video recordings of the locomotor paths were made with a digital video camera (Sentech STC-620CC), recorded with Final Cut Express 4.0 (Apple), converted with QuickTime 7.6.4 (Apple) and analyzed using DIAS 3.4.2 (Soli Technologies). For analysis of muscle size, dissected third-instar larvae were fixed and stained with Alexa Fluor-labelled phalloidin, and the area of muscle 6 in hemisegment A3 was measured.

smn mutants: used were an outcrossed stock of the SMN missense mutation smn^{73Ao} (Chan et al., 2003) (Gift from Dr. Greg Matera, UNC) and Df(3L)smn^{X7}, a small deletion that completely removes the SMN coding region without perturbing nearby genes (Chang et al., 2008). U6atac mutant: the P-element insertion P(LacW)K01105 which has previously been validated to disrupt U6atac (Otake et al., 2002) was used. SMN target gene screen: SMN target genes were inhibited by driving transgenic RNAi (Dietzl et al., 2007) expression in all tissues using da-Gal4; UAS-Dcr2 (Dietzl et al., 2007; Perrin et al., 2003); pan-neuronally using C155-Gal4; UAS-Dcr2 (Lin and Goodman, 1994) and in muscles using G14-Gal4. stas mutants: the stas P-element insertion mutant P{EPgy2}CG8408[EY04008] (Bellen et al., 2004) was obtained from the Bloomington Drosophila stock center. UAS-Stas: Stasimon cDNA was amplified from LD12309 (DGRC) and cloned into pBID-UASC (Wang et al., 2012). The construct was inserted at the attP40 landing site on chromosome 2L by phiC31 transgenesis (Groth et al., 2004). Stasimon was expressed in all neurons using nsyb-Gal4 (Bushey et al., 2009), cholinergic neurons using Cha-Gal4 (Salvaterra and Kitamoto, 2001) and glutaminergic motor neurons using OK371-Gal4 (Mahr and Aberle, 2006).

Drosophila NMJ electrophysiology was performed as previously described (Imlach and McCabe, 2009). Rhythmic motor activity was recorded from muscle 6 of abdominal segment A1 using a semi-intact preparation where the brain and peripheral nerves remained intact (Imlach et al., 2012). For analysis of locomotion, 60-second video recordings of the locomotor paths were made with a digital video camera (Sentech STC-620CC), recorded with Final Cut Express 4.0 (Apple), converted with QuickTime 7.6.4 (Apple) and analyzed using DIAS 3.4.2 (Soli Technologies) (Imlach et al., 2012). For analysis of muscle size, dissected third-instar larvae were fixed and stained with Alexa Fluor-labelled phalloidin, and the area of muscle 6 in hemisegment A3 was measured (Imlach et al., 2012).

### Zebrafish Motor Axon Outgrowth

Transgenic Tg(mnx1:GFP) zebrafish that express GFP in ventrally projecting motor axons (Dalgin et al., 2011) were used in this study. One-two cell stage Tg(mnx1:GFP) embryos were injected with MO (Table S3) and RNA as previously described (McWhorter et al., 2003). Motor axons from each side of the embryo were scored at 28 hours post-fertilization and used to classify the embryo as severe, moderate, mild, or no defects based on number and type of motor axon abnormalities (Carrel et al., 2006).

Zebrafish and embryos were maintained at ˜28.5° C. and staged by hours or days post-fertilization (Westerfield, 1995). Transgenic Tg(mnx1:0.6hsp70:GFP)os26 zebrafish embryos that express GFP in ventrally projecting motor axons (Dalgin et al., 2011) and are referred to as Tg(mnx1:GFP) were injected with ˜4.5 ng of antisense morpholino oligonucleotides (MO) as previously described (McWhorter et al., 2003). The sequence of all the MOs used in this study is shown in Table S3. Control, smn and tdp43 MOs were previously described (Kabashi et al., 2011; McWhorter et al., 2003). A reticule was used to measure the bolus volume injected into each embryo. For over-expression of human Stasimon (STAS), the open reading frame (Accession number NM_{—}015012) cloned in pcDNA3.1 was amplified with forward primer SEQ ID NO.151: 5′-ATAGGATCCATATGGCGAAAGGCAGAGTC-3′ and reverse primer SEQ ID NO.152: 5′-AGCTCGAGAGCTTACTCAAATTTCTGCTTTAG-3′ and subcloned into the pCS2+ vector using BamHI and XhoI sites. The Bcl2 (Accession number NM_{—}001030253.2) cDNA construct in pCS2+ was a kind gift from Dr. Thomas Look (Langenau et al., 2005). Plasmid DNA was linearized with NotI and capped RNA was generated using the Sp6 mMESSAGE mMACHINE kit (Ambion, Austin, Tex.) following the protocol of the manufacturer. One-two cell stage Tg(mnx1:GFP) embryos were injected with ˜4.5 ng of the indicated MOs with or without 200-300 pg of synthetic STAS or Bcl2 RNAs using an MPPI-2 Pressure Injector (Applied Scientific Instrumentation, Eugene, Oreg.). Injections of MO and RNA were performed according to previous protocols (Carrel et al., 2006). To visualize motor axons in GFP transgenic animals, live Tg(mnx1:GFP) zebrafish were anesthetized with tricaine, mounted on glass coverslips for observation using a Leica TCS-SL scanning confocal microscope. Ten motor axons from each side of the embryo were scored (total of 20 per embryo) at 28 hours post-fertilization and used to classify the embryo as severe, moderate, mild, or no defects according to previously described criteria based on number and type of motor axon abnormalities (Carrel et al., 2006). At least 3 independent sets of injections were performed per condition and 18-25 embryos (360-500 motor axons) scored in each experiment. The exact percentage of severe, moderate, mild, or no defects is dependent on the batch of smn MO and thus may be different in this report than in previously published reports (Carrel et al., 2006; McWhorter et al., 2003; Oprea et al., 2008).

### SMA Mice and Laser Capture Microdissection

FVB.Cg-Tg(SMN2*delta7)4299Ahmb Tg(SMN2)89Ahmb Smn1tm1Msd/J (JAX Stock No:005025) mice were interbred to obtain SMA-Δ7 (Smn−/−; SMN2+/+; SMNΔ7+/+) mice (Le et al., 2005). Tissues from control and SMA mice were rapidly dissected, immediately frozen in liquid nitrogen and stored at −80° C. until use. To label motor neurons and proprioceptive neurons of the motor circuit, Alexa 488-conjugated CTb was injected in the iliopsoas muscle of control and SMA-Δ7 mice at P2. Spinal cord and DRG were rapidly dissected at P6 and processed by LCM or immunohistochemistry.

### SMA Mice and Laser Capture Microdissection

All animal procedures were approved by the Institutional Laboratory Animal Care and Use Committee of Columbia University. FVB.Cg-Tg(SMN2*delta7)4299Ahmb Tg(SMN2)89Ahmb Smn1tm1Msd/J (JAX Stock No:005025) mice were interbred to obtain SMA-Δ7 (Smn−/−; SMN2+/+; SMN 7+/+) mice (Le et al., 2005). Genotyping of SMA mice was carried out as previously described (Gabanella et al., 2007).

Spinal cord and DRG from control and SMA mice injected in the iliopsoas muscle with CTb-488 were embedded in OCT, cryo-sectioned (10 μm) and adhered to UV treated PEN membrane slides (Leica). Tissue sections were immediately fixed in ethanol for 15 seconds prior to laser capture microdis section (LCM). LCM was carried out with the Leica DM6000B under 40× magnification to isolate CTb-488⁺ motor neurons from the L1-2 spinal cord segment and large (>20 μm) CTb-488⁺ proprioceptive neurons from L1-2 DRG of control and SMA mice. Immunohistochemistry and confocal microscopy of CTb-labeled tissues was performed to validate the identity of CTb-488⁺ neurons in the spinal cord as motor neurons (ChAT⁺) and in the DRG as proprioceptive neurons (parvalbumin⁺) as previously reported (Arber et al., 2000; Mentis et al., 2011).

### Lentiviral Constructs and Viral Production

With the exception of commercially available pLenti6/TR (Invitrogen), all other lentiviral constructs were generated by standard cloning techniques using the pRRLSIN.cPPT.PGK-GFP.WPRE vector (Addgene plasmid 12252) as a backbone (Dull et al., 1998; Zufferey et al., 1998). Schematic representations of these constructs are shown in FIG. 8A. The pLenti6/TR construct constitutively expresses the tetracycline-dependent repressor (TetR) protein under the control of the CMV promoter as well as the blasticidin resistance gene from the SV40 promoter. The pLenti.pur/Smn_{RNAi} construct expresses a shRNA targeting mouse SMN mRNA (SEQ ID NO.153: 5′-GAAGAAUGCCACAACUCCC-3′) under the control of a tetracyline-regulated H1_{TO} promoter as well as the puromycin resistance gene from the PGK promoter. The pLenti.hyg/SMN constitutively expresses an RNAi-resistant, epitope-tagged human SMN (Flag and Strep fused in tandem at the amino-terminus) under the control of the PGK promoter as well as the hygromycin resistance gene from the SV40 promoter. Viral stocks pseudotyped with the vesicular stomatitis G protein (VSV-G) were prepared by transient co-transfection of 293T cells using the ViraPower™ Lentiviral Packaging Mix (Invitrogen) following manufacturer's instructions.

### Antibodies and Western Blot

The following monoclonal antibodies were used in this study: anti-SMN clone 8 (BD Transduction Laboratories), Drosophila-specific anti-SMN (Chang et al., 2008), anti-SmB 18F6 (Carissimi et al., 2006), anti-B-actin (Sigma), anti-Tubulin DM 1A (Sigma), anti-FLAG (Sigma). For zebrafish experiments, an anti-hnRNP Q rabbit polyclonal antibody (Abcam), an anti-SMN mouse monoclonal antibody (2E6, gift from Dr. Glenn Morris) and an anti-β actin mouse monoclonal antibody (Santa Cruz) were used. For immunohistochemistry of mouse tissue, an anti-ChAT goat polyclonal antibody (Millipore) and an anti-parvalbumin chicken polyclonal antibody (Covance) were used.

Total protein extracts for Western blot analysis were prepared by homogenization of Drosophila third-instar larvae, zebrafish embryos or NIH3T3 cells in SDS sample buffer (2% SDS, 10% glycerol, 5% β-mercaptoethanol, 60 mM Tris-HC1 pH 6.8, bromophenol blue) followed by brief sonication and boiling. Protein concentration was measured using RC DC Protein Assay (Bio-Rad). All protein samples were analyzed by SDS/PAGE on 12% polyacrylamide gels followed by transfer onto nitrocellulose membrane and Western blot. SMN levels were determined relative to signal intensity in the serial dilution followed by normalization to Tubulin using ImageJ.

### Immunohistochemistry and Confocal Microscopy

For immunohistochemical analysis of mouse tissue, the spinal cord and the associated DRG (L1 and L2 spinal segments) were dissected under in vitro conditions and immersion fixed with 4% paraformaldehyde as previously described (Mentis et al., 2011). The spinal cord and DRG were harvested under cold (˜16° C.), oxygenated (95% O₂, 5% CO₂) artificial cerebrospinal fluid (128.35 mM NaCl, 4 mM KCl, 0.58 mM NaH₂PO₄.H₂O, 21 mM NaHCO₃, 30 mM D-Glucose, 0.1 mM CaCl₂.H₂O, and 2 mM MgSO₄.7H₂O). The tissue was immersion-fixed in 4% paraformaldehyde for 4 hours and then washed in 0.01 M phosphate buffer saline (PBS). Tissues were embedded in warm 5% Agar and serial transverse sections (70-80 μm) were cut on a Vibratome. The sections were blocked with 10% normal donkey serum in PBS-T (0.01 M PBS with 0.1% Triton X-100, pH 7.4) and incubated overnight at room temperature with a goat anti-ChAT polyclonal antibody (1:100) and a chicken anti-parvalbumin antibody (1:16,000). Secondary antibody incubations were performed for 3 hours with species-specific antisera coupled to either Rhodamine-RX or Cy5 (Jackson laboratories) diluted at 1:50 in PBS-T. After secondary antibody incubations, the sections were washed in PBS and mounted on slides with an anti-fading solution made of Glycerol:PBS (3:7). Sections were imaged using an SP5 Leica confocal microscope.

### RNA Analysis

Total RNA from Drosophila third-instar larvae, zebrafish embryos, mouse tissues and NIH3T3 cells was purified using TRIzol reagent (Invitrogen) and treated with RNase-free DNase I (Ambion) to remove DNA contamination. RNA from LCM neurons was purified using the Absolutely RNA Nanoprep Kit (Agilent) and linear amplification was performed with the MessageAmp II aRNA Amplification Kit (Ambion), according to the manufacturer's instructions. A mixture of oligo-dT primers and random hexamers was used to generate cDNA using Advantage RT-for-PCR kit (Clontech) and 1 μg of total RNA following manufacturer's instructions. For semi-quantitative RT-PCR analysis, 2.5% of the cDNA was used and PCR reactions were analyzed by electrophoresis on 1.5% agarose gels followed by staining with GelRed (Biotium). Data represent PCR reactions within the linear range of amplification as determined for each primer pair independently and in every set of experiments by sample collection every two cycles. The identity of all the PCR products was confirmed by DNA sequencing. For quantitative RT-qPCR analysis, 1% of the cDNA was used and each measurement was carried out in triplicates in a standard 3-step qPCR reaction with a Mastercycler ep Realplex⁴ (Eppendorf) PCR system and Power SYBR® Green PCR Master Mix (ABI). RT-qPCR data from NIH3T3 cells and Drosophila larvae were normalized to Gapdh and RpL32 (CG7939) mRNAs, respectively. For Northern blot analysis, total RNA from Drosophila third-instar larvae (2 μg) was analyzed by electrophoresis on 8% polyacrylamide/8M urea denaturing gel and transferred to a Hybond+ membrane (GE Healthcare). Radioactive antisense RNA probes against Drosophila snRNAs and 5.8S rRNA were transcribed in vitro from DNA oligonucleotide templates. Quantification was carried out using a Typhoon PhosphorImager (Molecular Dynamics). The list of primers and probes is shown in Table S3.

In Vitro snRNP Assembly and Analysis of snRNP Levels

NIH3T3 cell extracts were prepared by homogenization in ice-cold reconstitution buffer (20 mM Hepes-KOH pH 7.9, 50 mM KCl, 5 mM MgCl₂, 0.2 mM EDTA, 5% glycerol) containing 0.01% NP40 as previously described (Gabanella et al., 2007). Radioactive snRNAs were generated by run-off transcription with T7 polymerase from template DNA in the presence of [α³²-P] UTP (3000 Ci/mmol) and m7G cap analogue (New England Biolabs), and then purified from denaturing polyacrylamide gels. In vitro snRNP assembly experiments with radioactive snRNAs and NIH3T3 cell extracts (25 μg) followed by immunoprecipitation with anti-SmB antibodies were carried out according to established procedures (Gabanella et al., 2007; Gabanella et al., 2005; Pellizzoni et al., 2002; Workman et al., 2009). Quantification of immunoprecipitated snRNAs was carried out using a Typhoon PhosphorImager (Molecular Dynamics). For analysis of endogenous snRNP levels, NIH3T3 cell extracts (200 μg) were immunoprecipitated with anti-SmB antibodies in RSB-500 buffer (500 mM NaCl, 10 mM Tris-HCl pH 7.4, 2.5 mM MgCl₂) containing 0.1% NP40, EDTA-free protease inhibitor cocktail (Roche) and phosphatase inhibitors (50 mM NaF, 0.2 mM Na₃VO₄) for 2 h at 4° C. (Gabanella et al., 2007). After extensive washing with the same buffer, bound RNAs were recovered by proteinase K treatment, phenol/chloroform extraction and ethanol precipitation. The levels of snRNAs were measured using real-time RT-qPCR following the procedure previously described (Workman et al., 2009). The primers used are listed in Table S3.

### In Situ Hybridization

In situ hybridization of Drosophila embryos was performed as previously described (Kosman et al., 2004), with the following modifications. After incubation in anti-Dig-AP Fab fragments antibody (1:500, Roche), in situ experiments were developed for 10-30 minutes using the BCIP/NBT Alkaline Phosphatase Substrate Kit IV (Vector Labs). Antisense and sense Stasimon RNA probes were transcribed in vitro from PCR products amplified from the BDGP cDNA clone LD12309 (Rubin et al., 2000). The open reading frame of Drosophila Stasimon was amplified using the following forward SEQ ID NO.154: 5′-GATAATACGACTCACTATAGGGAGAGCTCGAAATTAACCCTCACTA-3′ and reverse SEQ ID NO.155 5′-GCAGATCTGATATCATCGCCACT-3′ primers for the sense probe, as a negative control, an antisense probe was amplified using the following forward SEQ ID NO.156: 5′-TGGCGGCCGCTCTAGAACTAG-3′ and reverse SEQ ID NO.157: 5′-GCTCGAAATTAACCCTCACTA-3′ primers.

### Statistical Analysis

Statistical analysis was carried out using two-tailed unpaired Student's t-test and the Prism 5 (GraphPad) software. Data are represented as mean and standard error of the mean (SEM) from independent experiments and P values are indicated as follows: *=p<0.05; **=p<0.01; ***=p<0.001. Statistical analysis of RNA expression in mouse tissues was carried out using one-way ANOVA followed by the Student-Newman-Keuls post hoc test and the asterisk (*) indicates p<0.05. For zebrafish motor axon characterization, the distribution of larval classifications (severe, moderate, mild, and no defects) was analyzed by comparing median values of each group with a two-tailed Mann-Whitney nonparametric rank test as previously described (Carrel et al., 2006).

### Example 2

### SMN Deficiency Causes U12 Splicing Defects in Mammalian Cells

Experiments were conducted to investigate whether there is an SMN requirement for U12 splicing based on the preferential reduction of minor snRNPs that occurs in SMA mice (Gabanella et al., 2007; Zhang et al., 2008). A mouse NIH3T3 cell line was used that allows doxycycline (Dox)-inducible, RNAi-mediated depletion of SMN. NIH3T3-Smn_{RNAi} cells cultured in the presence of Dox for 5 days showed strong knockdown of SMN mRNA (FIG. 8A-B) and reduction of SMN protein levels (FIG. 1A) compared to untreated cells. SMN deficiency severely decreased snRNP assembly of snRNAs in vitro and caused a profound alteration of their expression in NIH3T3 cells (FIGS. 1B and 8C-E), including a reduction in the levels of all Sm-class snRNPs of the U12 spliceosome. Importantly, expression of RNAi-resistant human SMN in NIH3T3-SMN/Smn_{RNAi} cells (FIG. 1A) rescued these changes (FIGS. 1B and 8C-D), indicating that they are SMN-dependent.

28 U12 introns were analyzed from 25 genes (Table S1) representing a diverse spectrum of features such as i) splice site subtype, ii) intron length, iii) intron position, iv) number of U12 introns, v) total number of introns, vi) evolutionary conservation, and vii) gene function. RT-PCR experiments showed a variety of U12 splicing defects in SMN-deficient NIH3T3-Smn_{RNAi} cells (FIGS. 1C and 8F). These defects included: i) accumulation of unspliced U12 introns in pre-mRNAs for Parp1, Nol1, Vps16 (both U12 introns 9 and 13) and C19orf54; ii) skipping of the two exons flanking the U12 intron in Clcn7, Tmem41b and Tspan3l mRNAs; and iii) aberrant splicing of Hars1 and Tmem4lb mRNAs. In the latter events, splicing of the U12 intron is bypassed through the use of an upstream cryptic 5′ splice site that is spliced to a downstream U2-dependent 3′ splice site. All of these splicing defects were rescued by expression of human SMN in NIH3T3-SMN/Smn_{RNAi} cells (FIG. 1C) and Dox treatment had no effects in control cells (FIG. 8F), demonstrating that the splicing defects are a direct consequence of reduced SMN levels. Increased U12 intron retention (FIG. 1D) as well as decreased levels of mature mRNAs (FIG. 1E) and accumulation of abnormally spliced mRNAs (FIG. 1F) in SMN-deficient NIH3T3 cells were confirmed by RT-qPCR. However, not all U12 introns were affected by SMN deficiency as no splicing defects were observed in 19 of the 28 U12 introns that were analyzed (FIG. 8F). U2 splicing in genes that also had U12 introns appeared to be normal, whether or not the U12 introns in these genes were affected by SMN deficiency, as did the splicing of several mouse genes with only U2 introns (FIGS. 8F-G and 9A). These results established that SMN deficiency causes U12 splicing defects in mammalian cells, perturbing the expression of a subset of the genes with this type of intron.

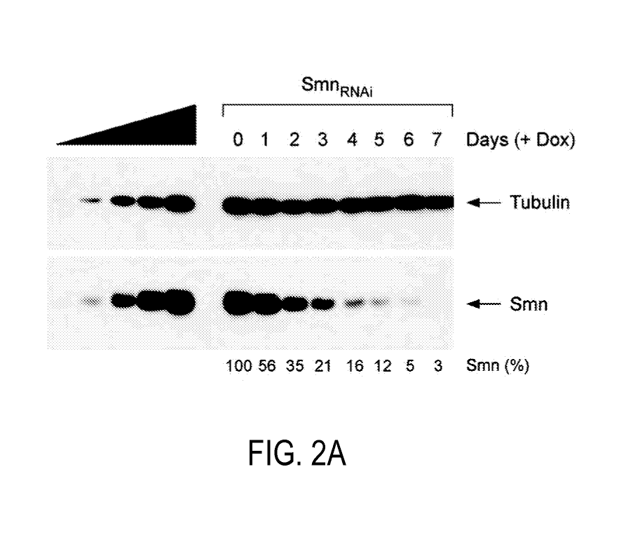

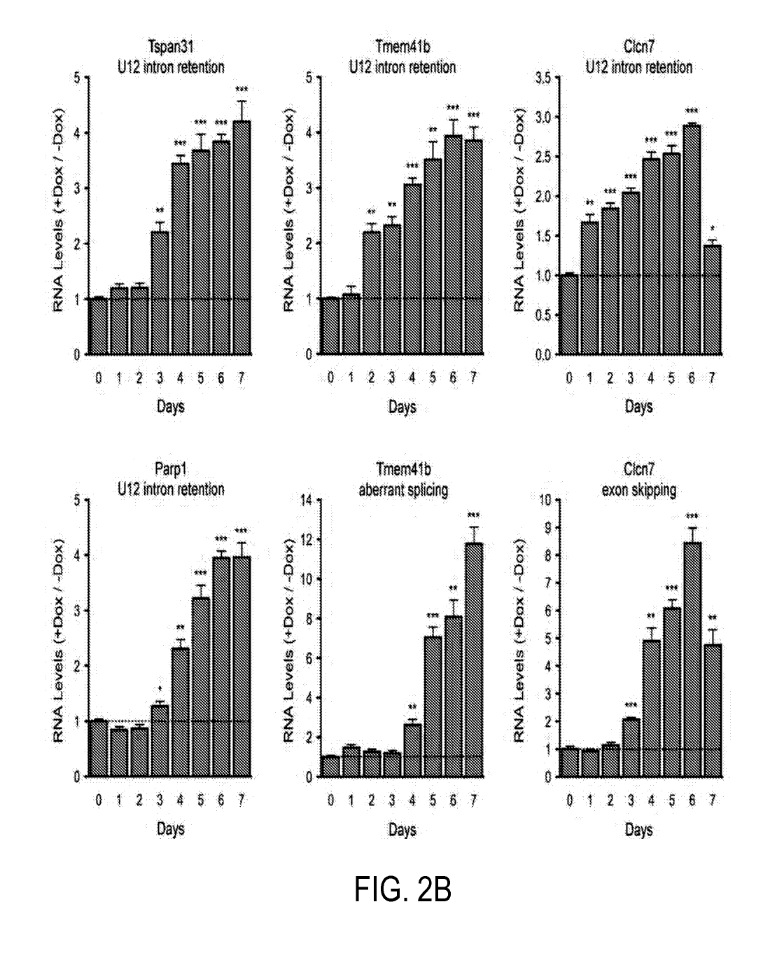

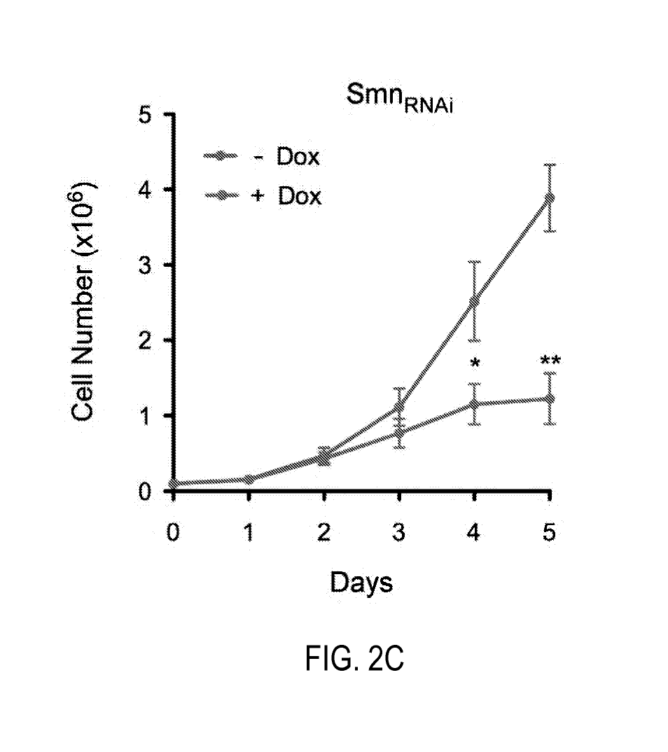

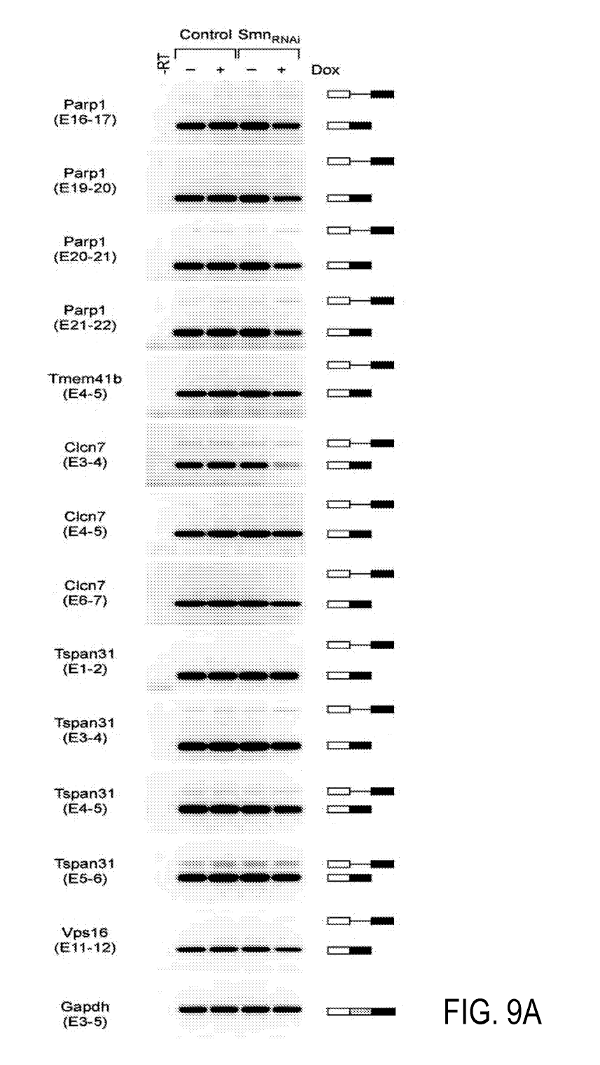

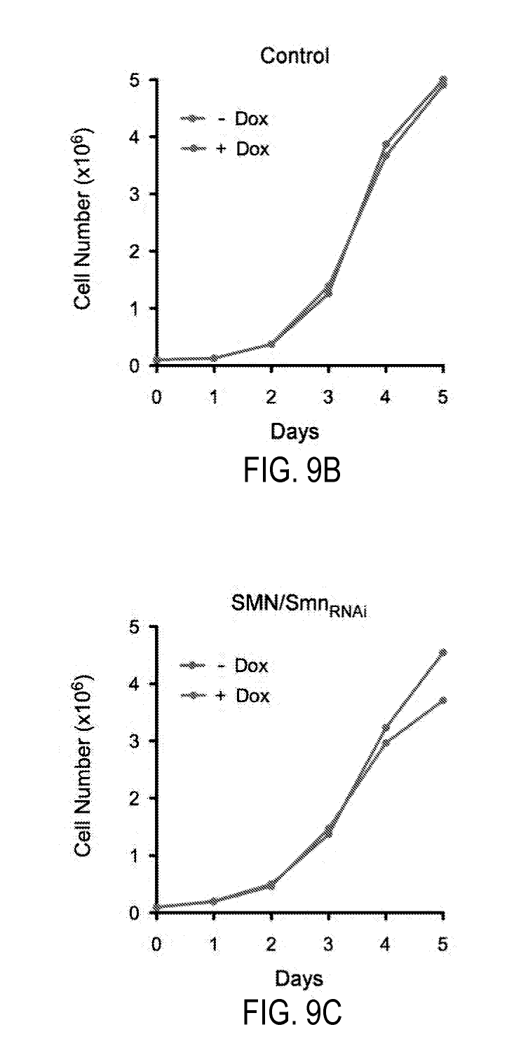

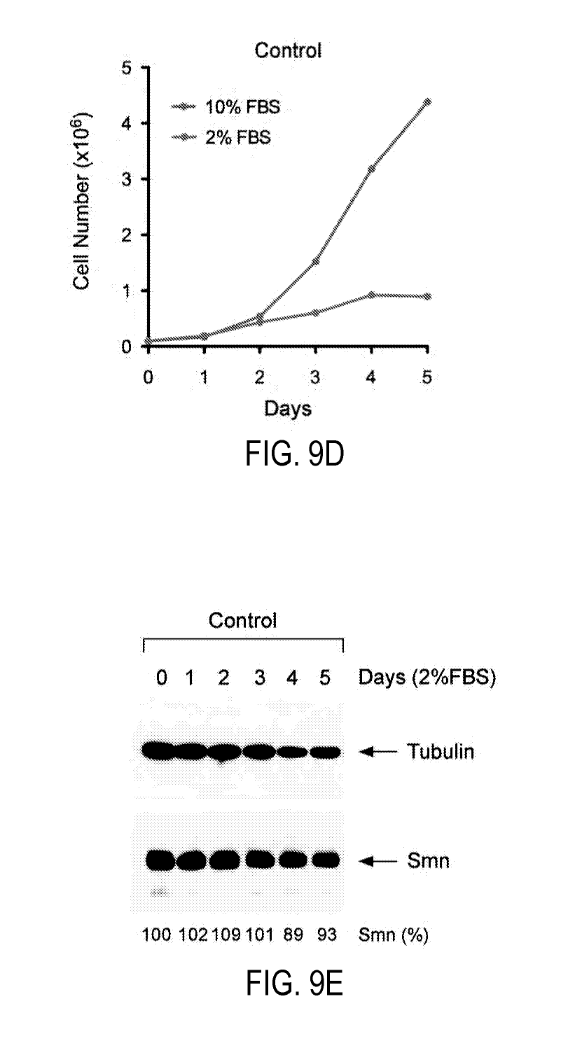

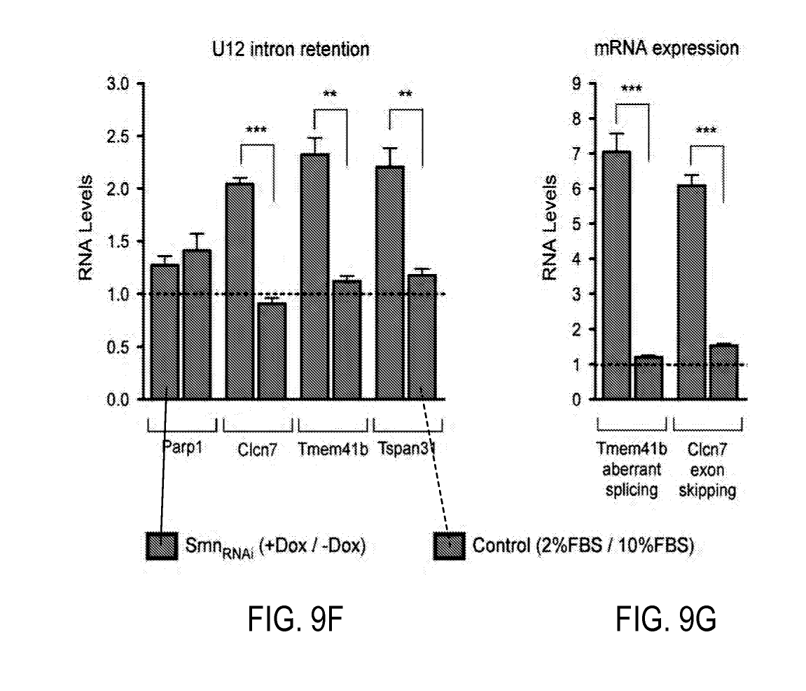

Defects in U12 splicing events directly regulated by SMN were expected to begin quickly after the onset of SMN reduction and increase in severity with progression of SMN depletion. A temporal analysis in NIH3T3-Smn_{RNAi} cells following Dox treatment over a period of 7 days was done. Analysis of representative target mRNAs showed that U12 intron retention began to accumulate in the first 3 days following RNAi induction and increased over time as SMN depletion progressed (FIG. 2A-B). Inefficient U12 intron splicing was accompanied by accumulation of aberrantly spliced Tmem41b and exon skipped Clcn7 mRNAs (FIG. 2B). The time of onset of these U12 splicing defects varied between genes indicative of a differential susceptibility of individual introns to SMN deficiency. SMN deficiency decreased the proliferation of NIH3T3-Smn_{RNAi} cells (FIG. 2C), while Dox alone had no effect in wild-type cells (FIG. 9B). This reduced proliferation was SMN-dependent as it was corrected by expression of human SMN in NIH3T3-SMN/Smn_{RNAi} cells (FIG. 9C). The occurrence of SMN-dependent U12 splicing defects was detectable prior to onset of this decrease in cell proliferation, indicating that these defects were not a consequence of reduced cell proliferation. In support of this, decreasing the proliferation of NIH3T3 cells through the reduction of serum levels (FIG. 9D) had no effect on SMN expression or on SMN-dependent U12 splicing events (FIG. 9E-G). Thus, SMN deficiency results in both the early production and the accumulative increase of U12 intron splicing defects in mammalian cells, consistent with regulation of these events by SMN.

### Example 3

### SMN is Required for Expression of snRNAs and U12 Intron-Containing Genes in Drosophila

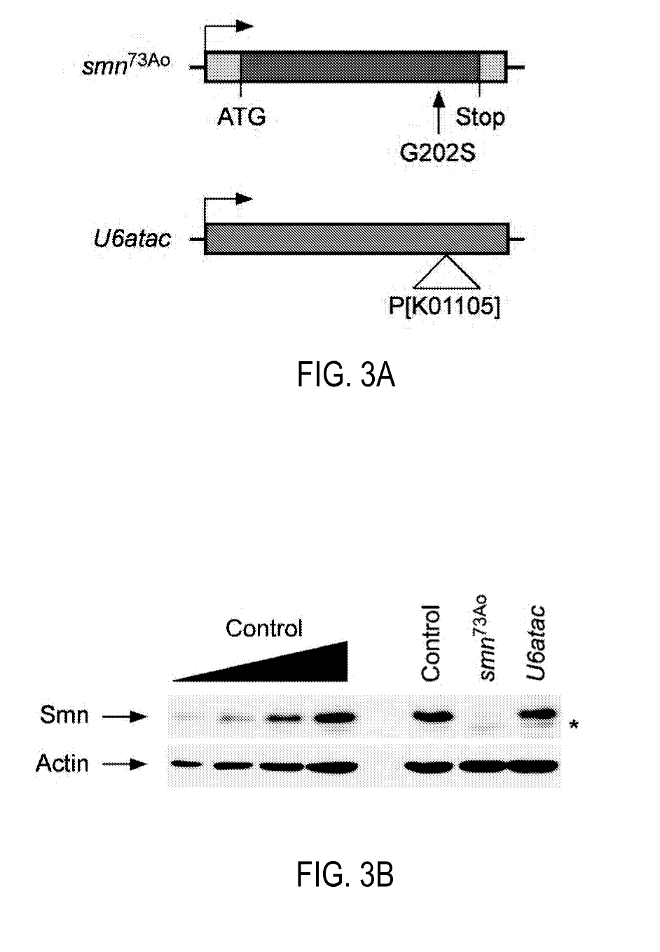

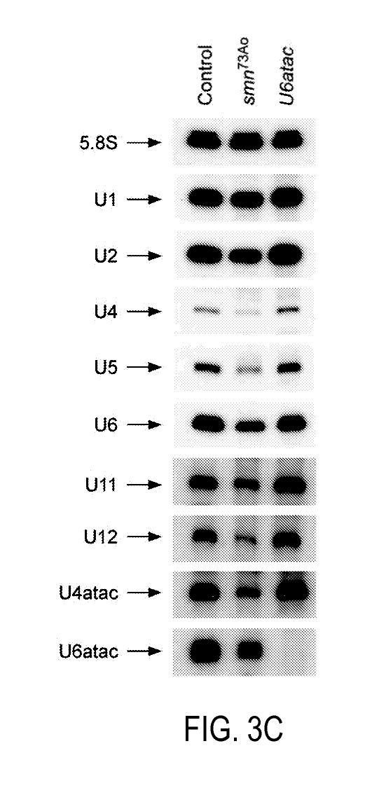

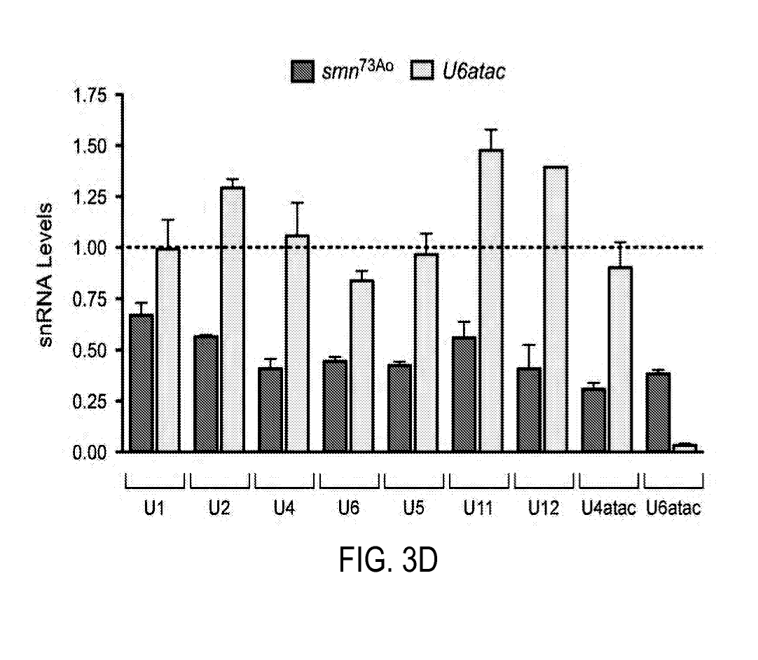

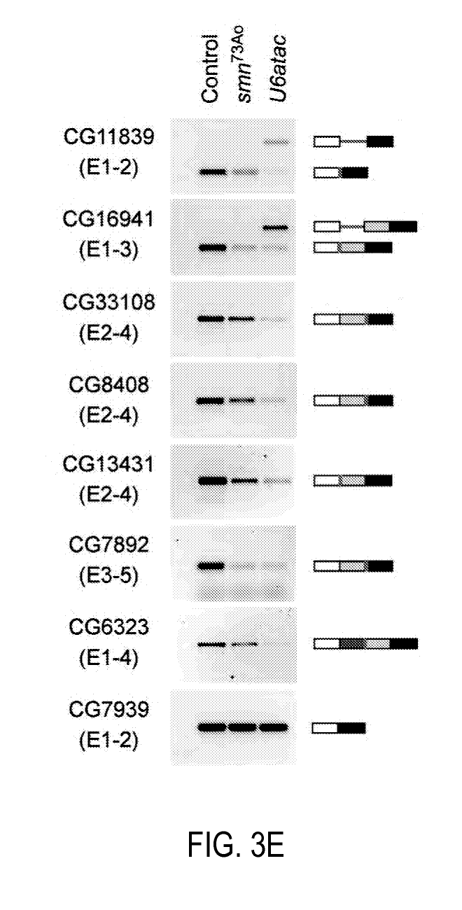

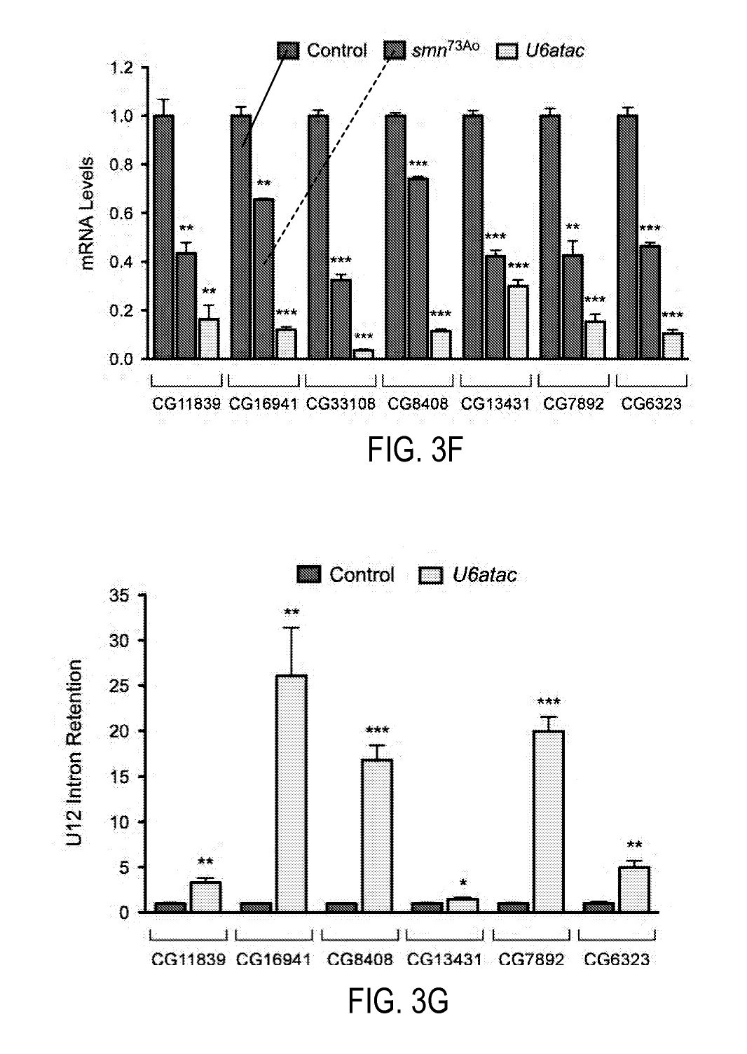

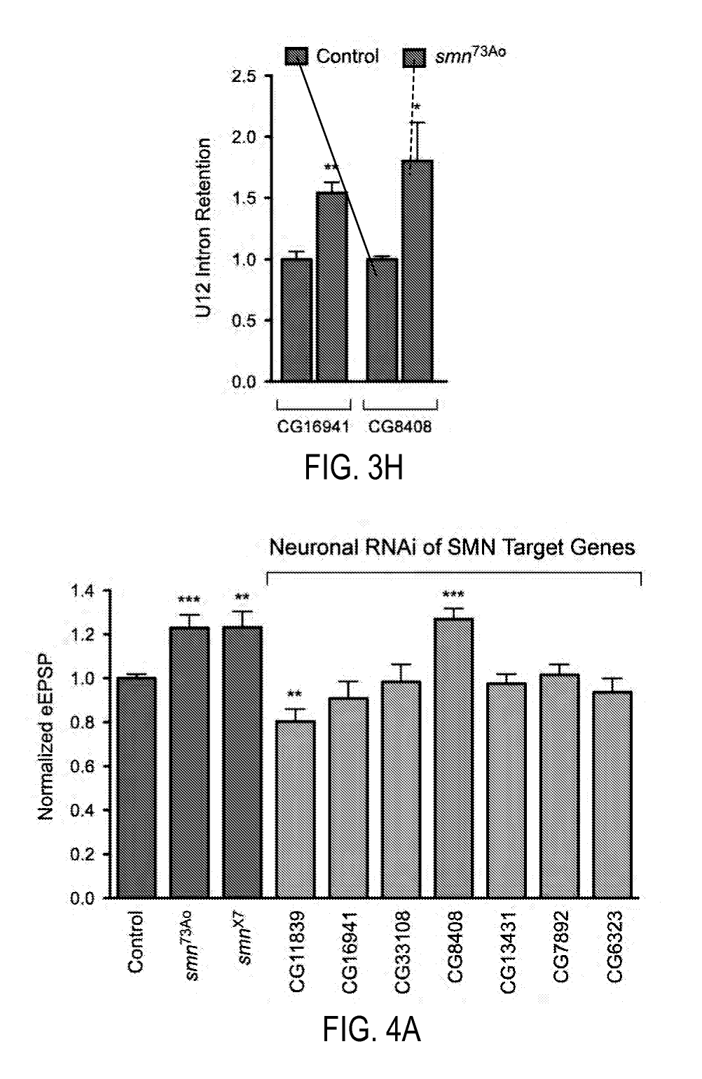

The genome-wide effects of SMN deficiency on U12 splicing in vivo, were determined using Drosophila for both the availability of genetic mutants and the presence of only 23 putative U12 introns in the genome of this organism (Alioto, 2007; Lin et al., 2010) (Table S2). Previously characterized loss-of-function smn^{73Ao} point mutant allele, which produces an unstable protein (Chan et al., 2003) (FIG. 3A) was used. As a control for intron excision by the U12 spliceosome, a mutant of the U6atac snRNA gene (U6atac^{K01105}), which specifically disrupts U12 splicing (Otake et al., 2002) (FIG. 3A) was used. As expected, there was a large reduction of SMN levels in smn^{73Ao} mutants but no change in U6atac^{K01105} mutants compared to wild-type third-instar larvae (FIG. 3B).

The effect of SMN deficiency on snRNA expression in Drosophila was studied using northern blot analysis of U6atac^{K01105} mutant larvae. The results showed that U6atac was depleted while other snRNAs appeared normal or slightly increased (FIG. 3C). In contrast, the levels of all the snRNAs were decreased in smn^{73Ao} mutants with differential effects of SMN deficiency on the accumulation of individual snRNAs ranging from a 70% reduction of U4atac to a 30% reduction of U1 (FIG. 3C-D). Thus, SMN is required for normal expression of spliceosomal snRNAs in Drosophila.

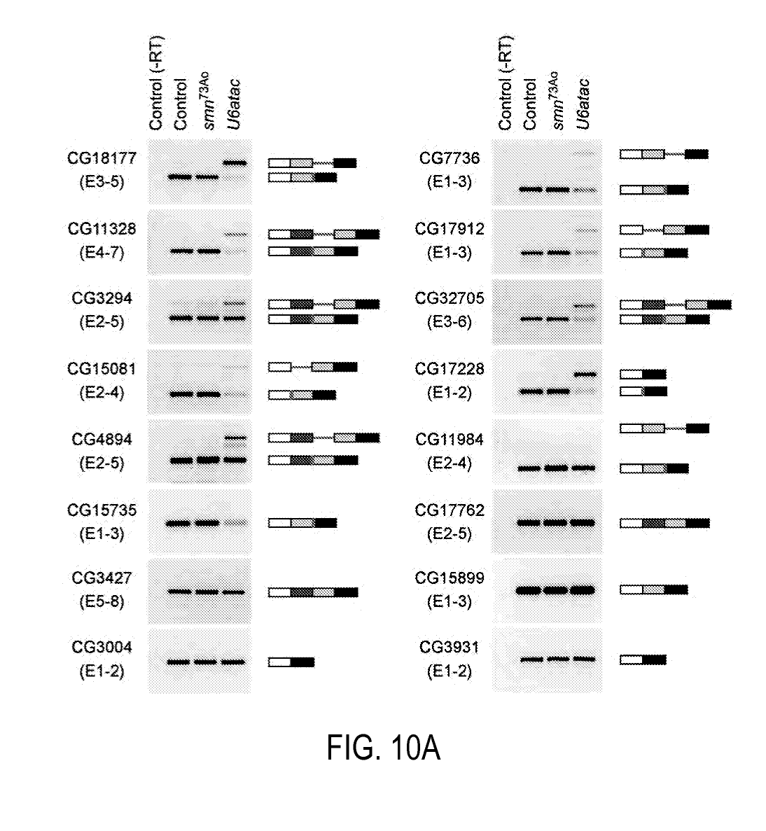

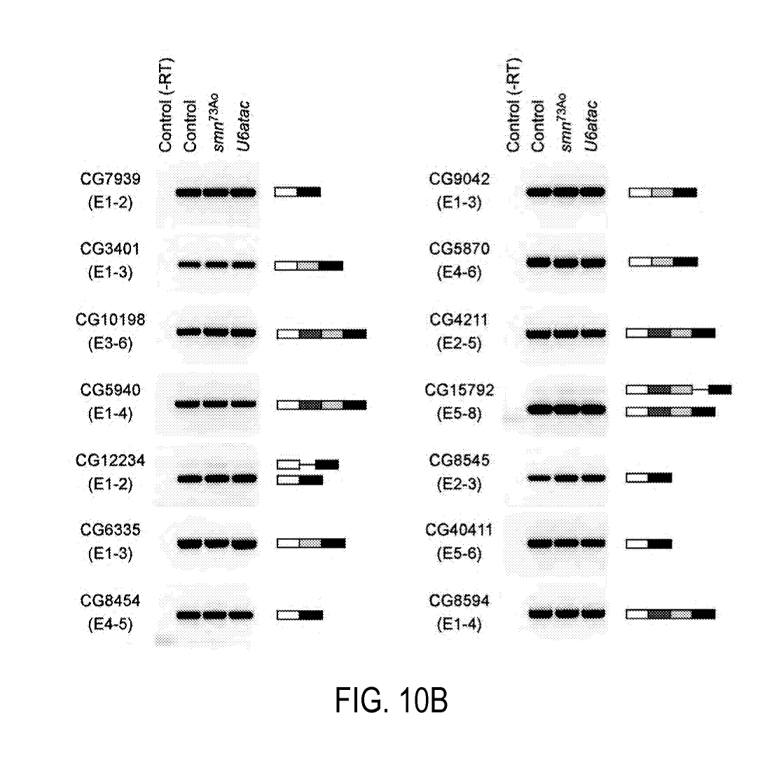

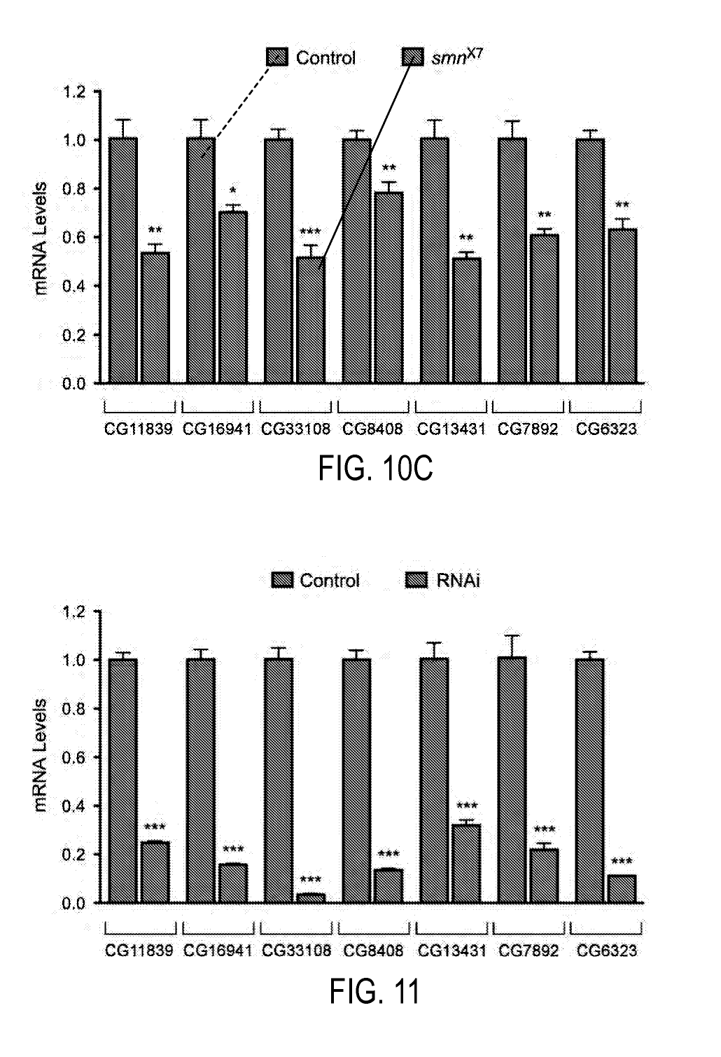

The effects of SMN deficiency and decreased snRNA levels on U12 splicing in Drosophila were next analyzed for expression and splicing of all predicted U12 intron-containing genes by RT-PCR. This genome-wide analysis showed a decrease in the levels of spliced mRNAs or an increase of pre-mRNAs containing unspliced U12 introns in 18 of the 23 predicted U12 intron-containing genes in U6atac^{K01105} larvae (FIGS. 3E and 10A), validating the presence of U12 introns in these 18 Drosophila genes. Remarkably, the mRNA levels of 7 of the 18 U12 intron-containing genes were also decreased in smn^{73Ao} mutants (FIG. 3E). RT-qPCR experiments using smn^{73Ao} larvae as well as a second Drosophila smn mutant allele (smn^{X7}) which has a complete deletion of the smn gene (Chang et al., 2008) confirmed that the expression of these 7 genes was SMN-dependent (FIGS. 3F and 10C). No changes in mRNA expression or U2 splicing were detected in the remaining U12 intron-containing genes or in additional genes that contained only U2 introns (FIG. 10A-B). These experiments showed that SMN is required for normal expression of ˜40% of all the U12 intron-containing genes in the Drosophila genome.

The levels of U12 intron retention in both U6atac^{K01105} and smn^{73Ao} mutants were measured by RT-qPCR. Consistent with inefficient U12 splicing, there was increased U12 intron retention for 6 of these genes in U6atac^{K01105} mutants and for 2 of these genes in smn^{73Ao} mutants (FIG. 3G-H). Notably, the mammalian homologs of 3 of the 4 Drosophila genes that were down-regulated in smn mutants and have evolutionarily conserved U12 introns (Tmem41b/CG8408, Tspan31/CG6323 and C19orf54/CG33108) also had defective U12 intron splicing in SMN-deficient NIH3T3 cells (FIG. 1). The expression and splicing of 5 genes (CG8594/Clcn7, CG8545/Nol1, CG40411/Parp1, CG6335/Harsl and CG8454/Vps16) that have only U2 introns in Drosophila were studied. These five genes have a U12 intron in the homologous mouse genes and they were perturbed by SMN deficiency in NIH3T3 cells (FIG. 1C-E). The expression and splicing of all 5 of these genes appeared normal in Drosophila smn mutants (FIG. 10B). Furthermore, 3 genes (CG13431/Mgat1, CG16941/Sf3a1, CG11839/Znf830) that have both U12 introns and reduced expression in Drosophila smn mutants but only have U2 introns in the mouse homolog had normal expression in SMN-deficient NIH3T3 cells (FIGS. 3E and 8G). Thus, SMN deficiency affects the expression of U12 intron-containing genes in Drosophila and some of these SMN-dependent U12 splicing events are conserved across evolution.

### Example 4

### Stasimon is an SMN Target Gene Required for Normal Synaptic Transmission of Drosophila Motor Neurons

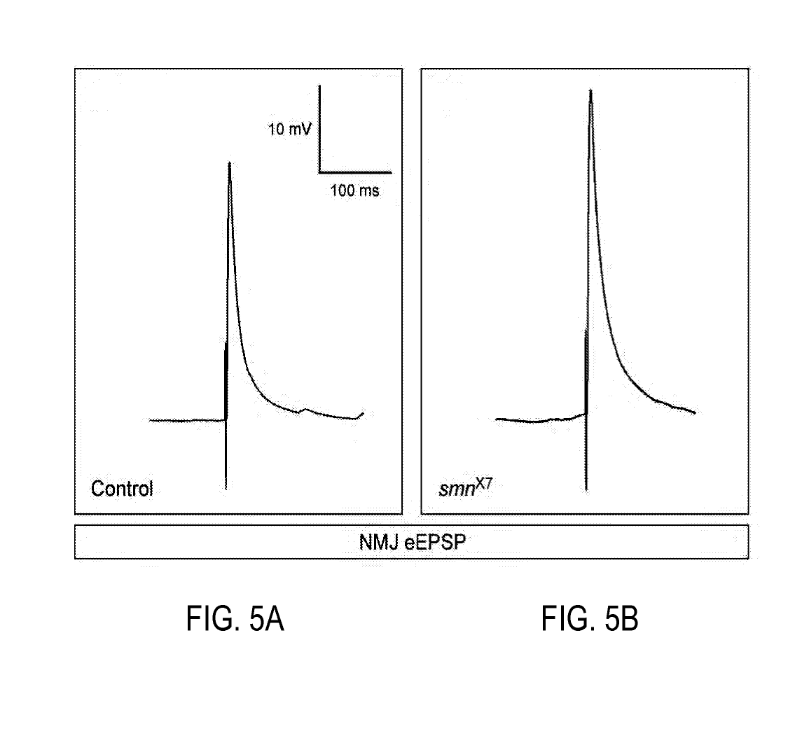

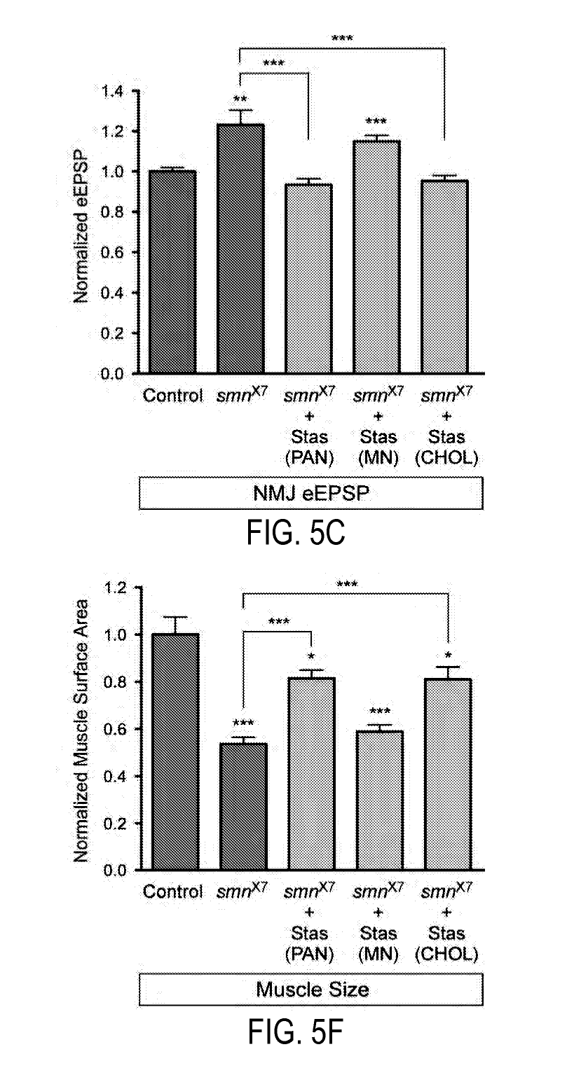

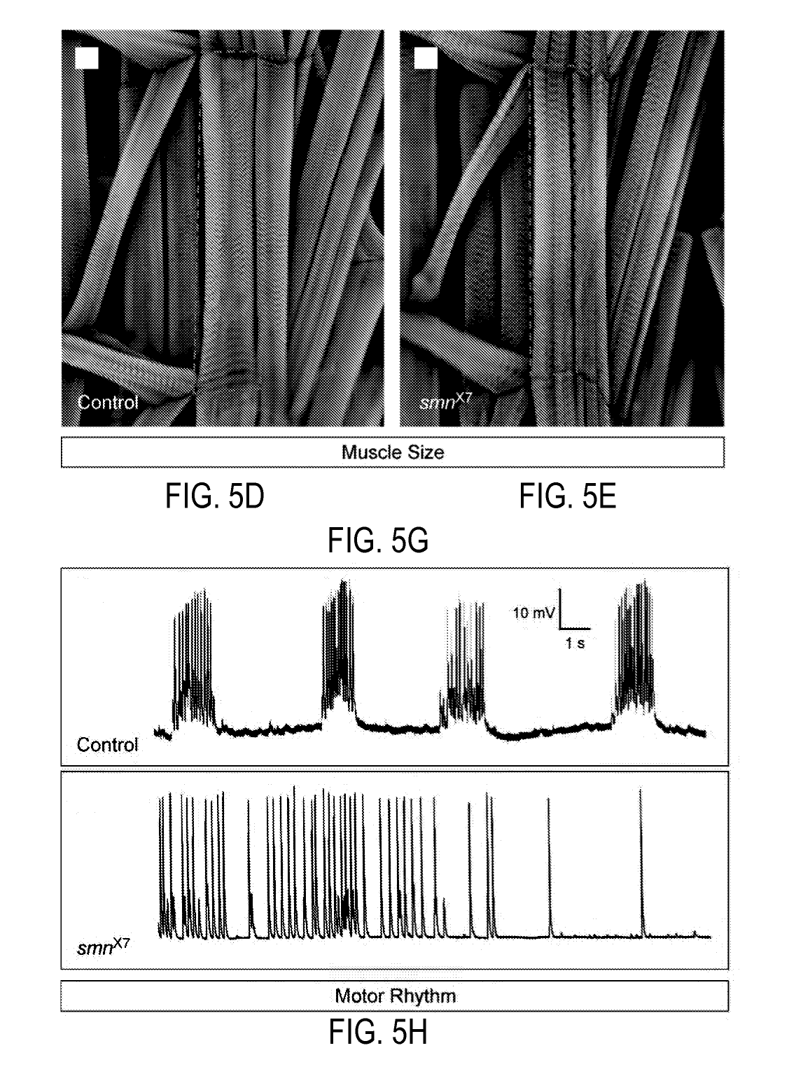

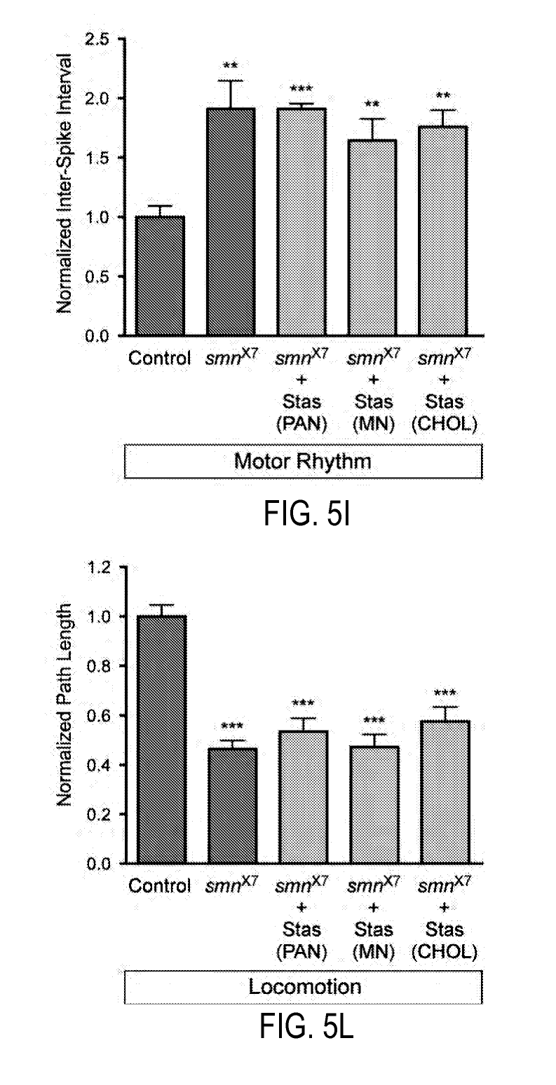

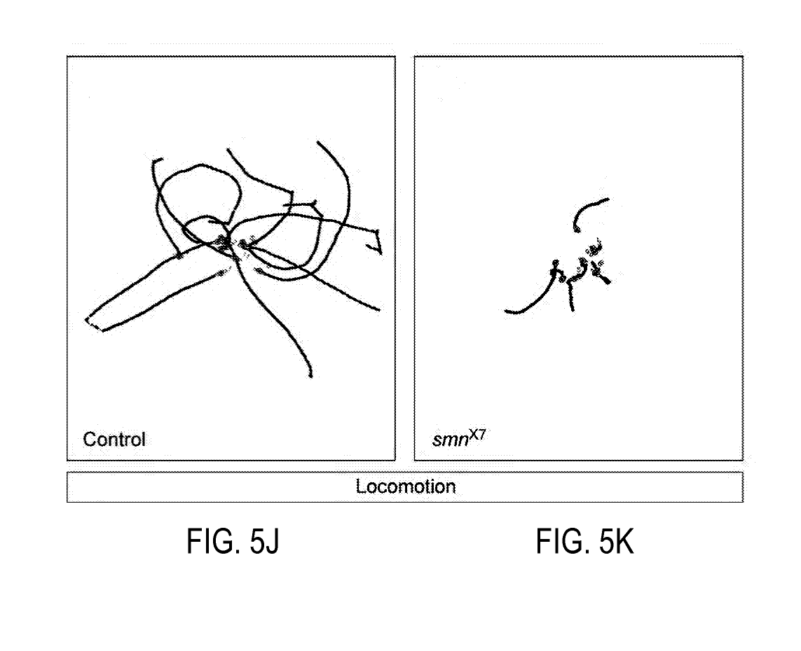

The identification of the subset of Drosophila genes with U12 introns that are regulated by SMN provided the foundation for functional analysis of their role in motor neurons. Drosophila smn⁷³⁰ and smn^{x7} mutants have an aberrant increase in evoked Excitatory Post-Synaptic Potential (eEPSP) amplitudes at the neuromuscular junction (NMJ) to ˜125% of controls (FIGS. 4A and 5A-B) that is rapidly corrected by transgenic SMN expression in neurons (Imlach et al., 2012), consistent with this phenotype being an early consequence of SMN reduction.

RT-qPCR confirmed that transgenic UAS-RNAi constructs (Dietzl et al., 2007) were able to potently decrease the expression of their target mRNA in Drosophila larvae (FIG. 11). The effect of pan-neuronal knockdown of each of these genes on NMJ neurotransmitter release was studied. Strikingly, knockdown of CG8408 resulted in an increase in NMJ eEPSP amplitudes to 127% of controls, similar to the evoked neurotransmitter release defects observed in smn mutants (FIG. 4A). In contrast, knockdown of CG11839 decreased eEPSP amplitudes at the NMJ (80.3% of control), unlike Drosophila smn mutants and therefore not pursued further, while knockdown of the 5 other SMN target genes had no effect on neurotransmitter release compared to controls (FIG. 4A). As CG8408 and its mouse homologue Tmem41b (FIG. 1) are both U12 intron-containing genes whose expression is regulated by SMN and knockdown of CG8408 in Drosophila neurons causes an electrophysiological phenotype similar to that of smn mutants, the CG8408 gene was renamed stasimon (stymied in smn)—abbreviated as stas.

Homologs of Stasimon are found in humans, mice and zebrafish in addition to other species (FIG. 12A), and display a remarkably high degree of amino acid conservation although their function is unknown. The Stasimon protein is predicted to contain six transmembrane domains and a SNARE-associated Golgi protein domain (FIG. 12B). Furthermore, in situ hybridization showed strong expression of Stasimon mRNA in the nervous system of Drosophila embryos as well as in the mouse spinal cord (FIG. 12C-D) (Lein et al., 2007), consistent with a prominent role in neurons.

### Example 5

### Stasimon is Required in Cholinergic Neurons for Normal Synaptic Transmission of Drosophila Motor Neurons

Stasimon expression was decreased in subsets of Drosophila neurons by RNAi and the effects on synaptic transmission of motor neurons were analyzed. Surprisingly, Stasimon knockdown in motor neurons did not change eEPSP amplitudes compared to controls (FIG. 4C-D), suggesting that Stasimon is not cell autonomously required in glutaminergic motor neurons for this phenotype. In contrast, Stasimon knockdown in cholinergic neurons produced an increase in eEPSP amplitudes at motor neuron terminals (122% of control) remarkably similar to that of pan-neuronal Stasimon knockdown (FIG. 4C-D). These results demonstrated that reduction of Stasimon perturbs the neurotransmitter release properties of motor neurons indirectly through disruption of the activity of cholinergic neurons in the motor circuit. Importantly, analogous NMJ neurotransmission defects in Drosophila smn mutants are caused by SMN deficiency in cholinergic neurons but not in motor neurons (Imlach et al., 2012).

### Example 6

### Stasimon Expression Rescues Synaptic Dysfunction in Drosophila smn Mutants

Stasimon expression was restored by pan-neuronal transgenic expression of Stasimon cDNA in smn^{X7} mutants and then neurotransmitter release at the NMJ was measured. Strikingly, restoring Stasimon expression in all neurons completely rescued the NMJ eEPSP amplitude of smn mutants to control levels (FIG. 5A-C). This was consistent with the reduction of Stasimon function causing the neurotransmission defects at the NMJ of Drosophila smn mutants.

Similar to pan-neuronal Stasimon expression, transgenic Stasimon expression in the cholinergic neurons of Drosophila smn^{x7} mutants fully corrected NMJ eEPSP amplitudes to control levels (FIG. 5C). In contrast, expression of transgenic Stasimon in the glutaminergic motor neurons of smn^{X7} mutants did not alter NMJ eEPSP amplitudes (FIG. 5C). Thus, similar to SMN (Imlach et al., 2012), Stasimon expression must be restored in cholinergic neurons that provide excitatory input to motor neurons in order to rescue defects in NMJ neurotransmission in Drosophila smn mutants.

Reduced muscle size, decreased locomotion and altered rhythmic motor activity are additional phenotypes of Drosophila smn mutants (FIG. 5D-L), all of which are rescued by SMN expression in cholinergic neurons (Imlach et al., 2012).

The effects of Stasimon restoration in the neurons of Drosophila smn mutants showed that muscle surface area in smn^{X7} mutants was reduced by 50% compared to controls (FIG. 5D-F). Remarkably, pan-neuronal restoration of Stasimon in smn^{X7} mutants resulted in a robust increase in muscle area to ˜80% of that in control larvae (FIG. 5F). Expression of Stasimon in the cholinergic neurons but not in the motor neurons of smn^{X7} mutants produced an identical increase in muscle area to pan-neuronal expression (FIG. 5F). These results indicated that Stasimon deficiency contributes to the muscle growth phenotype in Drosophila smn mutants. In contrast, locomotion and rhythmic motor activity were not significantly improved by pan-neuronal, motor neuron or cholinergic neuron expression of Stasimon in Drosophila smn^{X7} mutants (FIG. 5G-L). Thus SMN-dependent, decreased expression of Stasimon in cholinergic neurons accounts for the dysfunction of neurotransmitter release at the NMJ and contributes to defects of muscle growth in Drosophila smn mutants.

### Example 7

### Stasimon Expression Rescues SMN-Dependent Motor Neuron Defects in Zebrafish

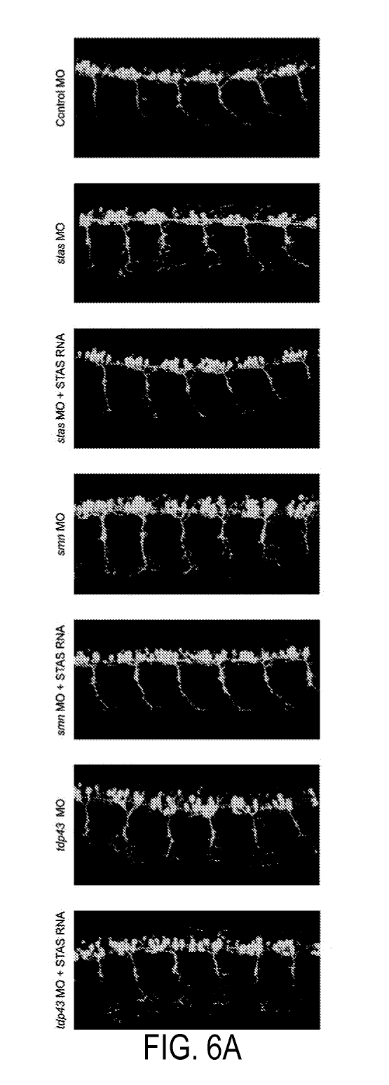

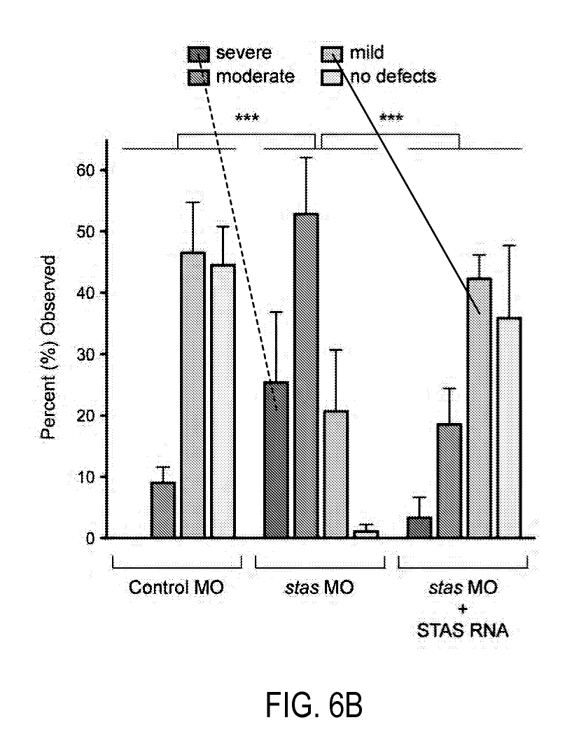

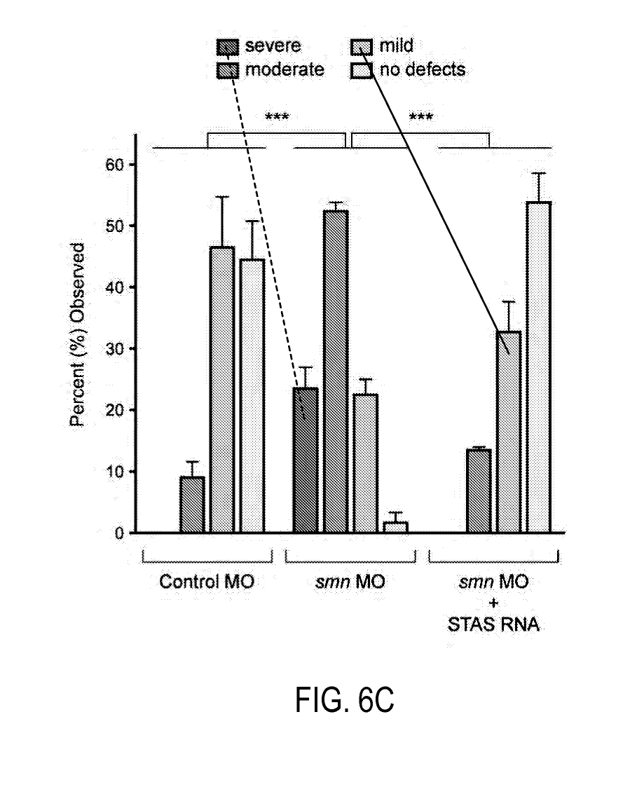

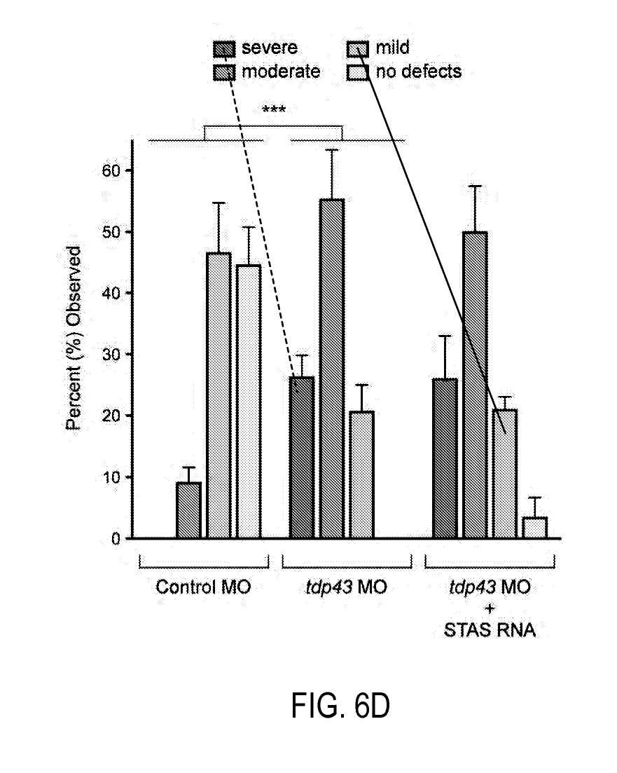

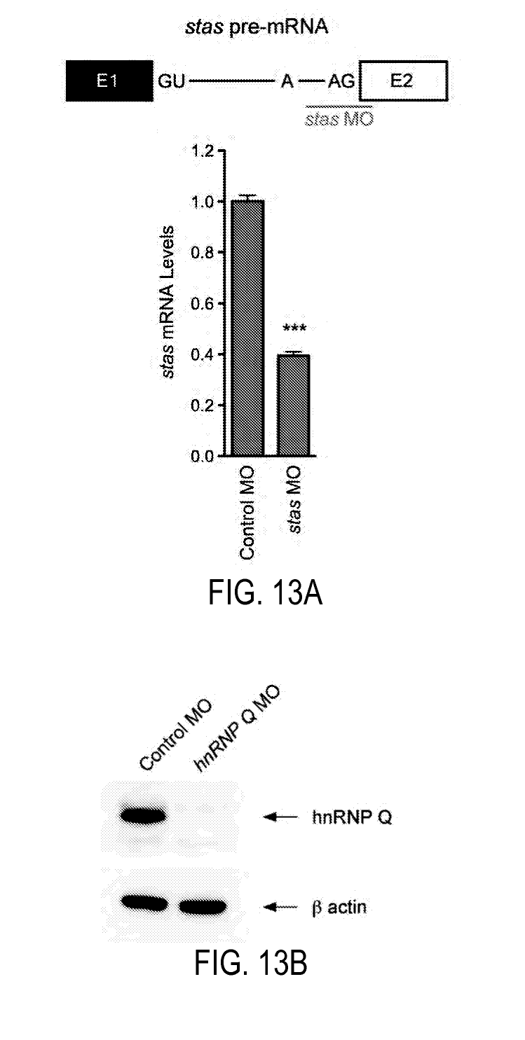

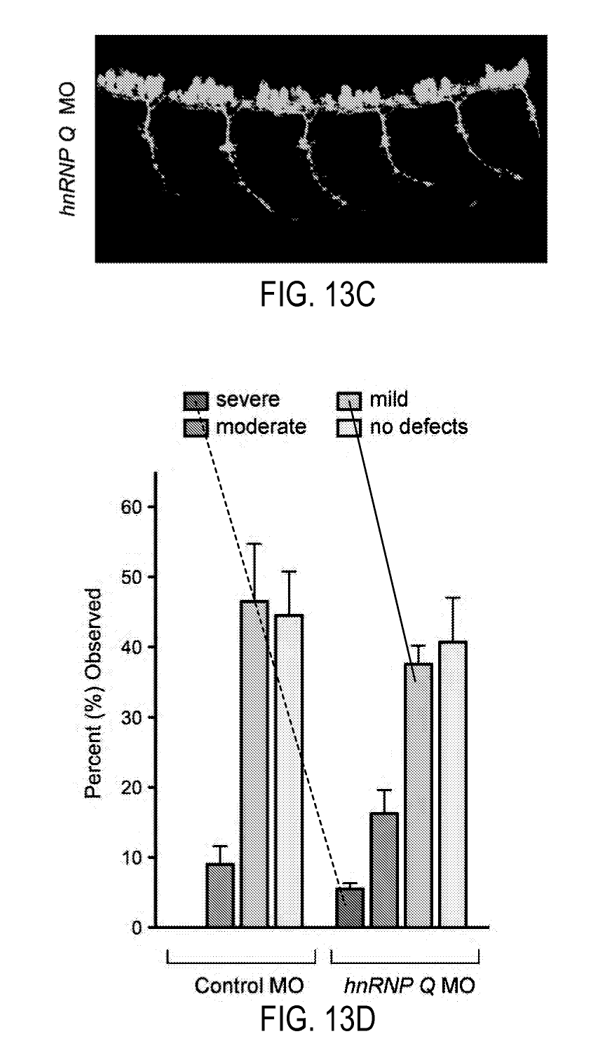

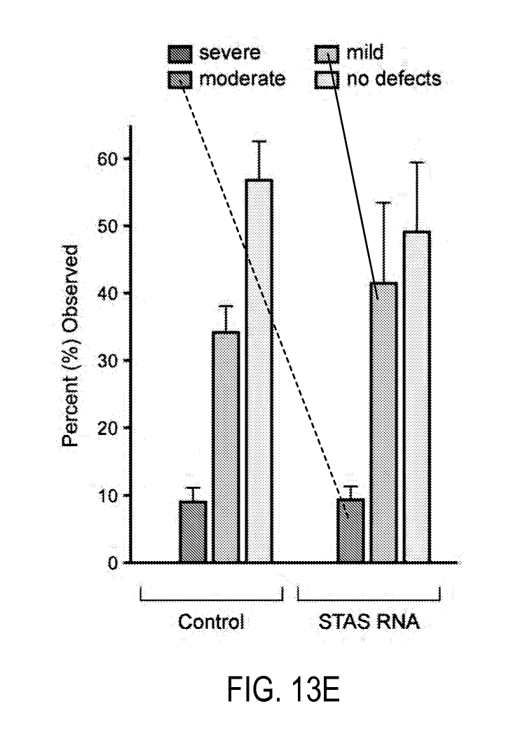

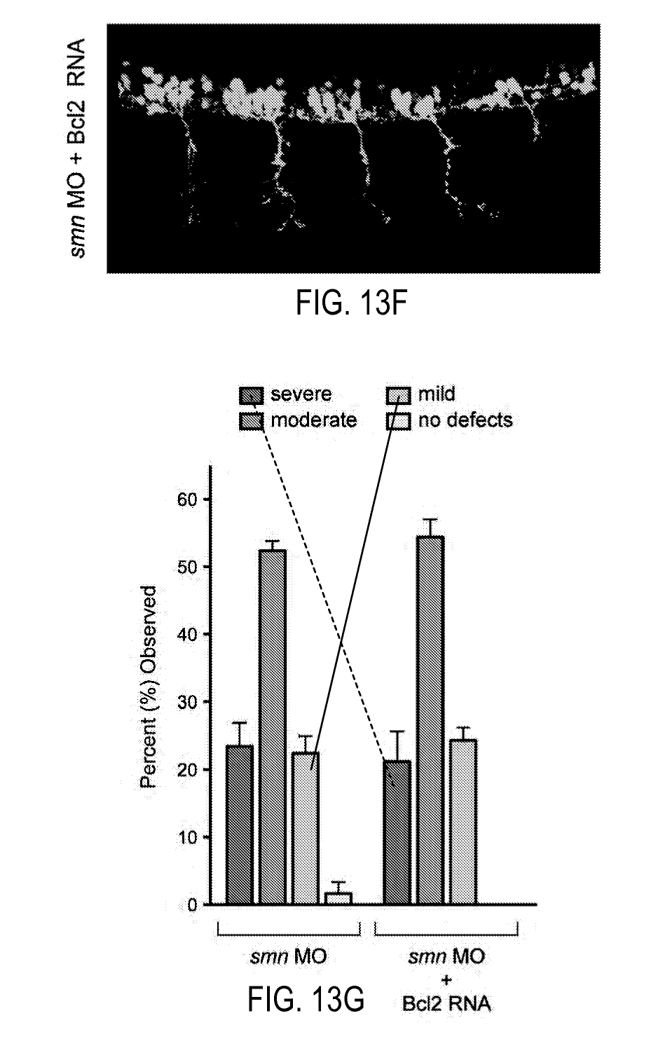

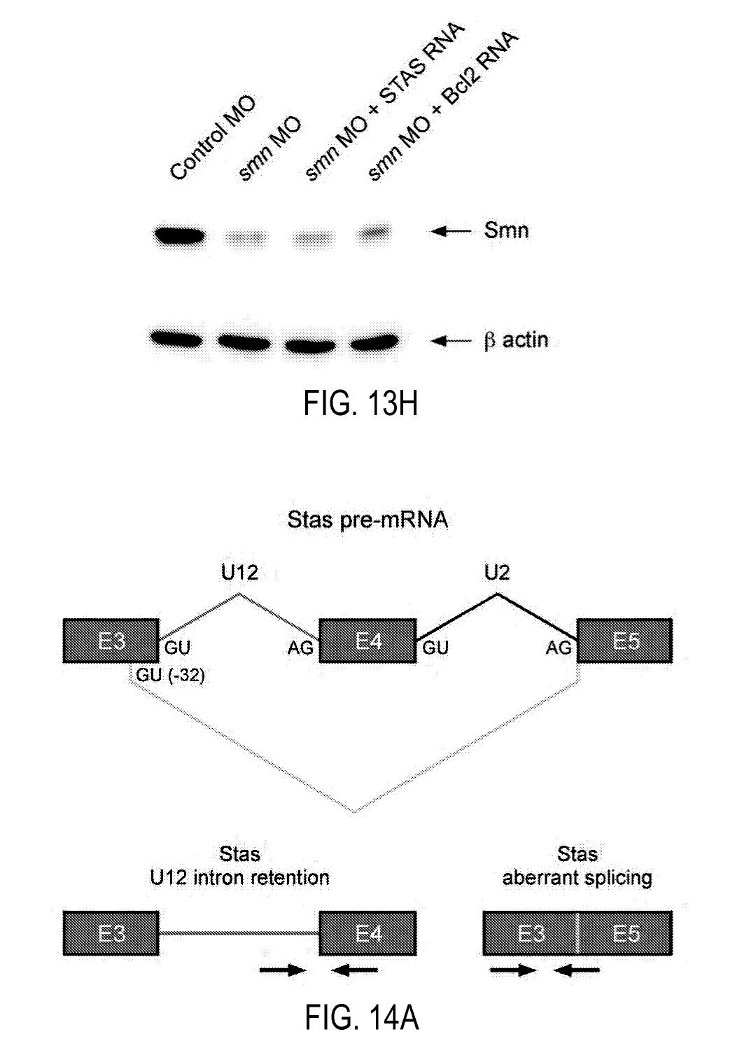

A vertebrate model of SMN deficiency (Beattie et al., 2007) is the knockdown of SMN in zebrafish embryos, which causes developmental defects in motor neuron axonal outgrowth that include truncations and abnormal branching (McWhorter et al., 2003), and that can be corrected by injection of mRNAs encoding wild-type human SMN but not SMN mutants associated with SMA (Carrel et al., 2006). The requirement for Stasimon for motor neuron development was studied by decreasing Stasimon expression with an antisense morpholino oligonucleotide (MO) in a zebrafish transgenic line expressing GFP in motor neurons (FIG. 13A). This decrease severely disrupted the normal outgrowth of motor axons without other detectable alterations in the morphology of developing embryos (FIG. 6A-B). Injection of a control MO caused no significant change of motor axons (FIG. 6A-B). Furthermore, while injection of human Stasimon mRNA alone had no significant effects on normal motor axon development (FIG. 13E), co-injection of this mRNA rescued the motor axon defects induced by stas MO injection (FIG. 6A-B). These results established that Stasimon is required for normal motor neuron development in zebrafish.

Consistent with previous studies (Carrel et al., 2006; McWhorter et al., 2003), injection of smn MO caused severe motor axon abnormalities compared to control embryos (FIGS. 6A and 6C) while knockdown of a control RNA binding protein did not (hnRNP Q, FIG. 13B-D). Remarkably, co-injection of Stasimon mRNA with smn MO resulted in a robust correction of the

SMN-dependent motor axon defects as measured by a strong reduction in the degree of abnormal motor axon branching (FIGS. 6A and 6C). This was not observed upon injection of an unrelated control mRNA (Bcl2, FIG. 13F-G) and SMN levels were similarly reduced by the smn MO with and without co-injection of either Stasimon or control mRNAs (FIG. 13H), indicating that mRNA co-injection did not interfere with the activity of the smn MO.

To determine if the rescuing effect of Stasimon was specific to SMN-dependent phenotypes in zebrafish, the effect of Stasimon on motor neuron phenotypes induced by depletion of the amyotrophic lateral sclerosis associated gene TDP43 (Kabashi et al., 2011) was studied. Injection of tdp43 MO in zebrafish embryos induced motor axonal defects (FIGS. 6A and 6D) as previously reported (Kabashi et al., 2011). However, co-injection of human Stasimon mRNA had no beneficial effects on the TDP43-dependent motor neuron phenotypes (FIGS. 6A and 6D). Collectively, these results established that increasing Stasimon expression can specifically rescue neuronal dysfunction in this vertebrate model of SMN deficiency.

### Example 8

### SMN Deficiency Disrupts Stasimon U12 Splicing and mRNA Expression in the Motor Circuit of SMA Mice

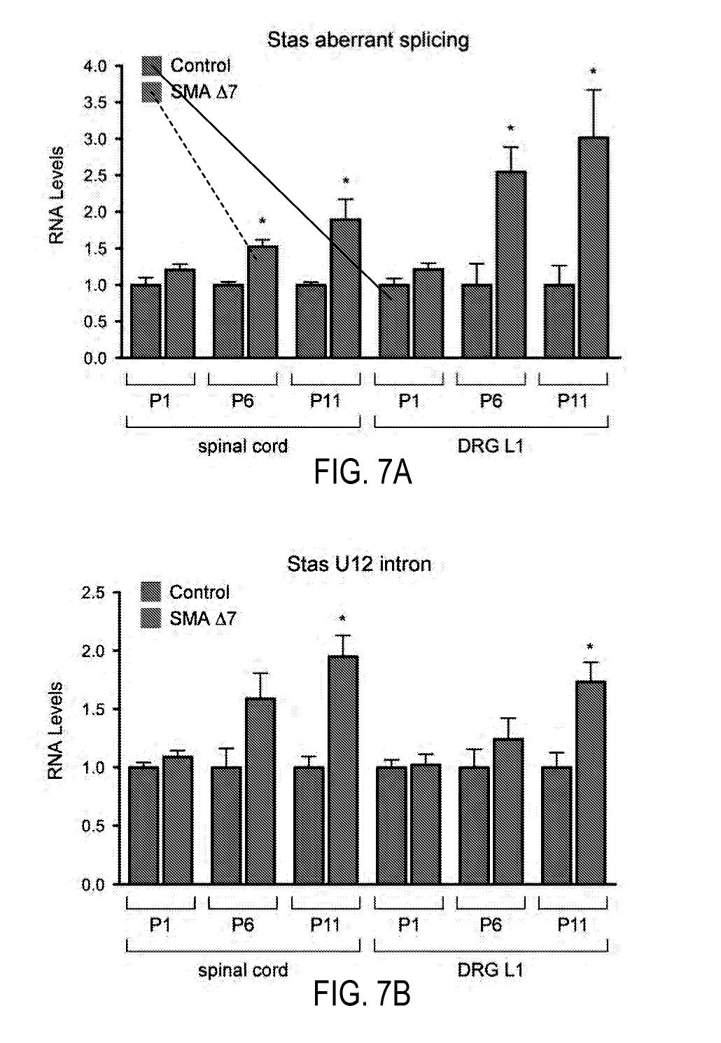

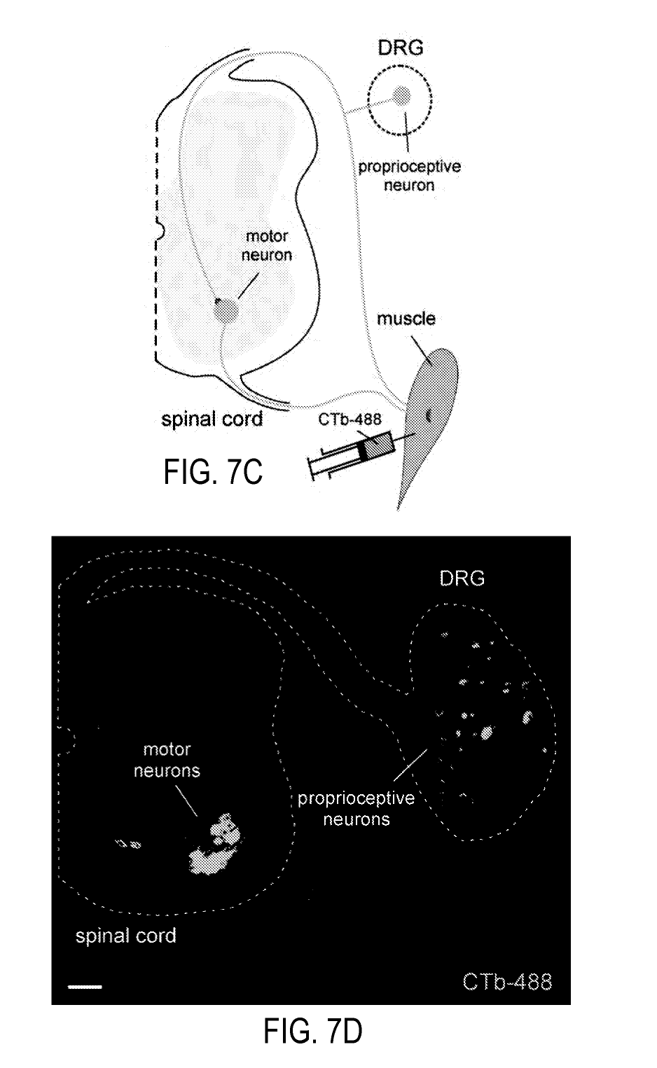

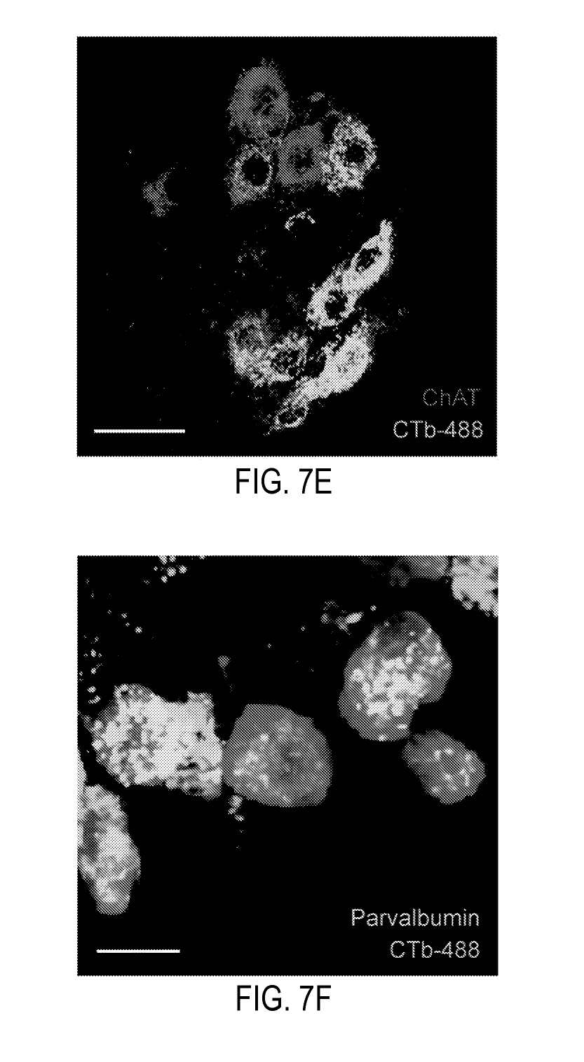

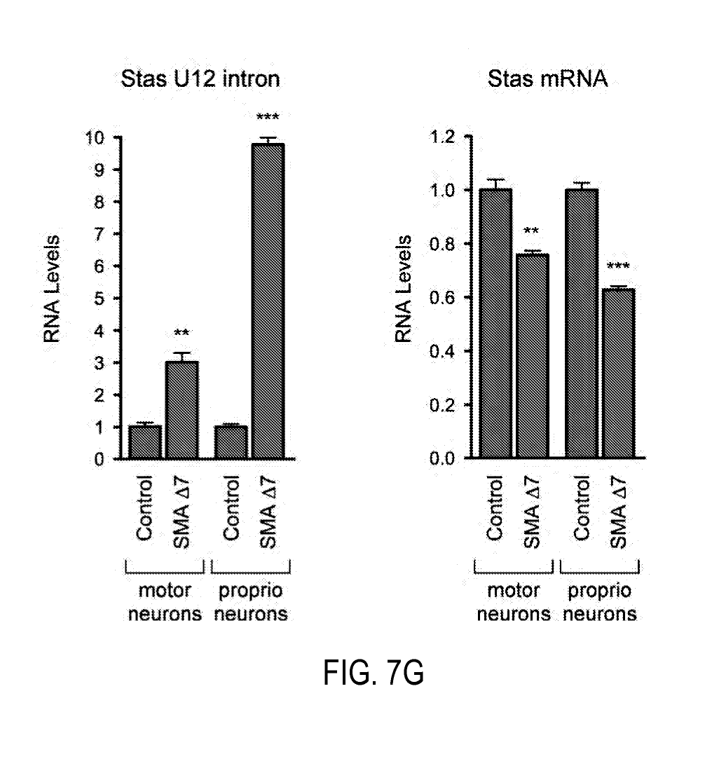

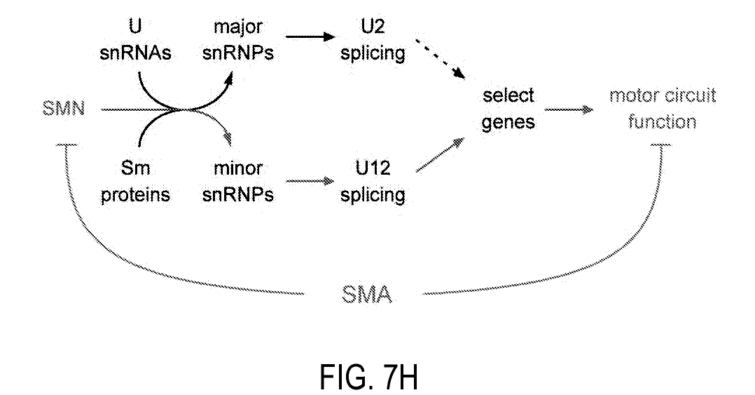

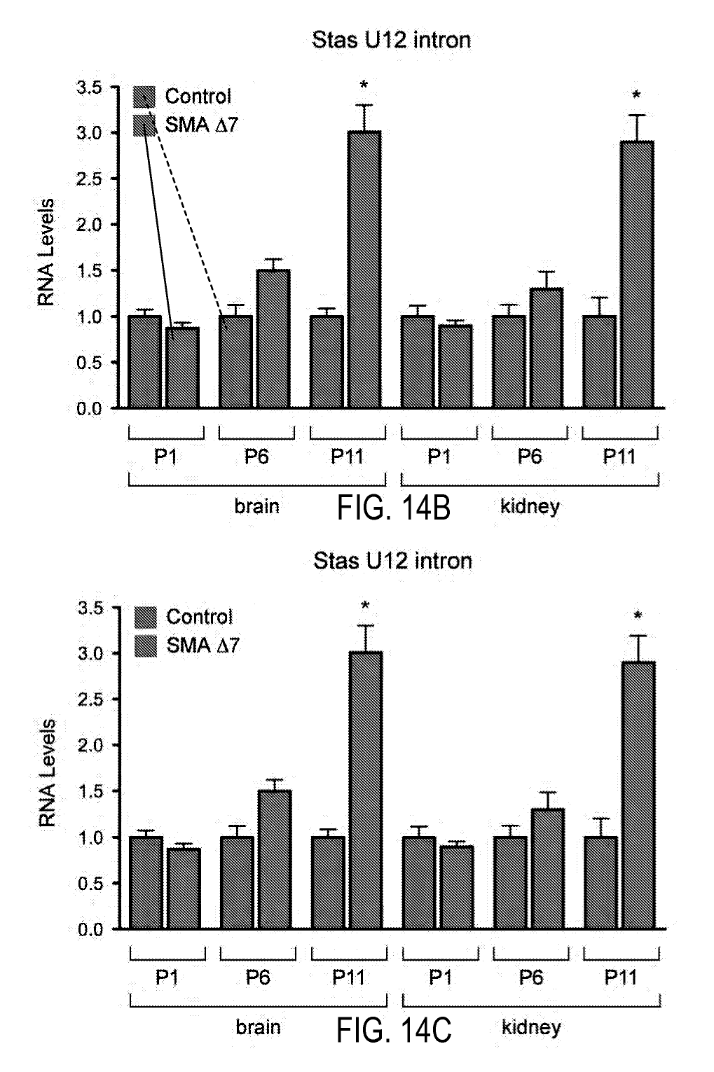

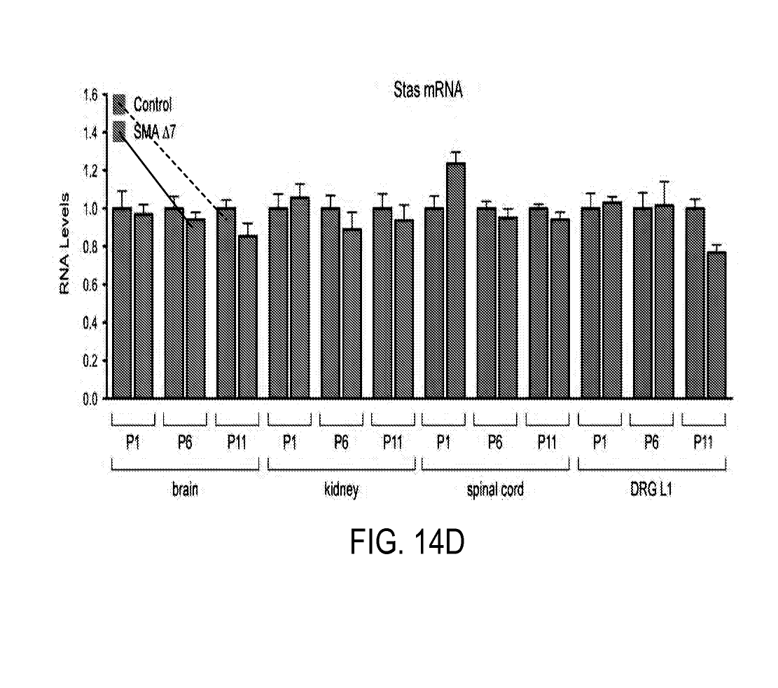

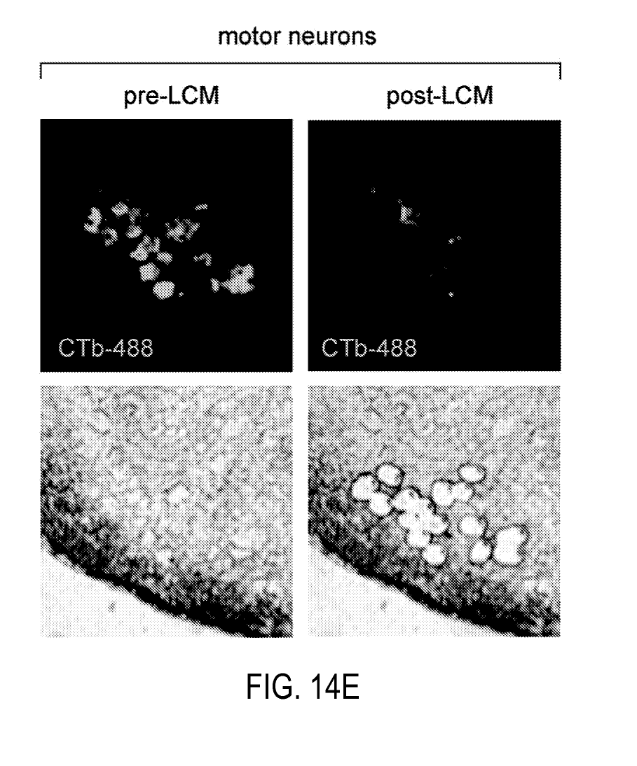

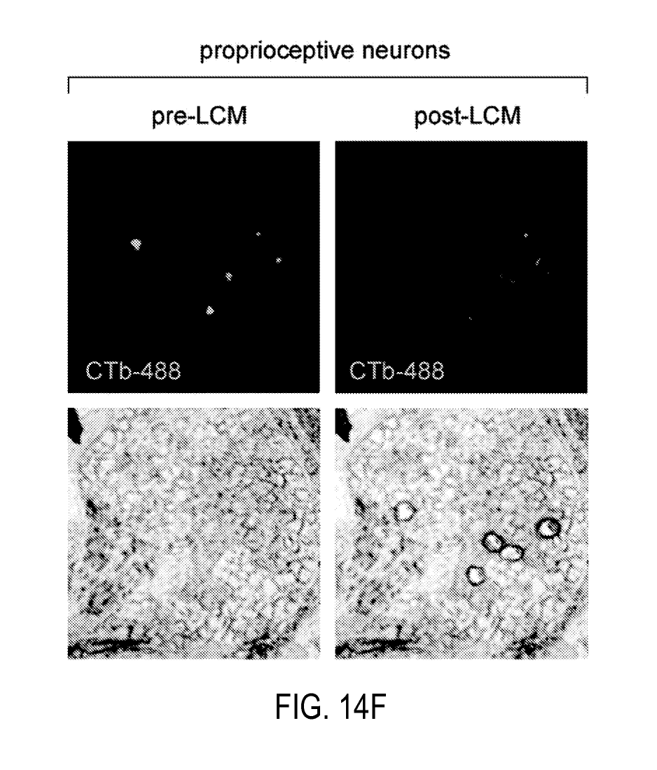

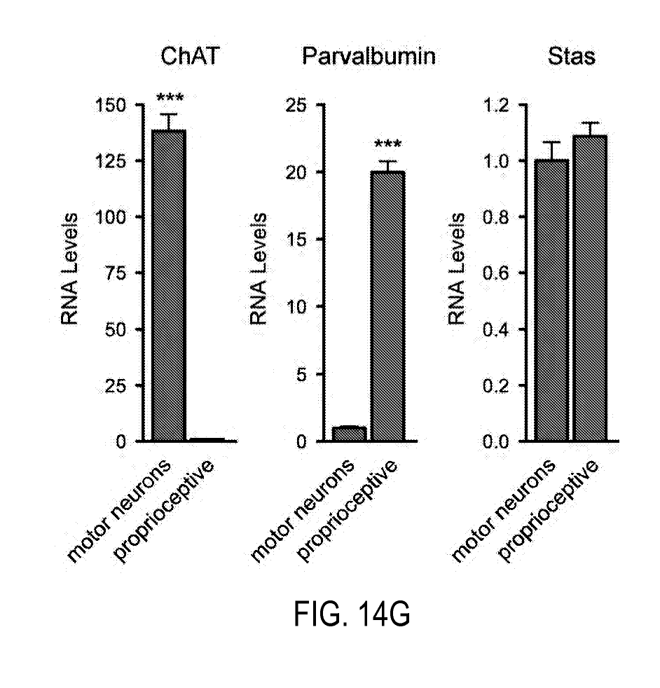

The mouse SMA model (SMNΔ7) recapitulates many features of the human disease (Le et al., 2005). Stasimon mRNA splicing and U12 intron retention were analyzed by RT-qPCR (FIG. 14A) in tissues of SMNΔ7 SMA and control mice at postnatal day 1 (P1), P6 and P11, corresponding to pre-, early- and late-symptomatic stages of disease in this model. While there was no apparent difference in Stasimon U12 splicing at P1, aberrantly spliced Stasimon mRNA was detectable in the spinal cord and first lumbar Dorsal Root Ganglia (L1 DRG) of SMNΔ7 SMA mice compared to controls at the early-symptomatic P6 stage (FIGS. 7A-B and 14B-C). Stasimon aberrant splicing further accumulated in late-symptomatic P11 SMNA7 SMA mice and was accompanied by increased levels of U12 intron retention (FIG. 7A-B). Significant accumulation of Stasimon U12 intron retention but not of the aberrantly spliced mRNA was also found in brain and non-neuronal kidney tissue (FIG. 14B-C). These results established that SMN deficiency caused a progressive alteration in Stasimon U12 splicing in the spinal cord and DRG of SMA mice.

Stasimon mRNA levels in brain, spinal cord, DRG and kidney of SMNΔ7 SMA mice were not changed compared to normal littermates (FIG. 14D), with the exception of a reduction of Stasimon mRNA in L1 DRG of SMNΔ7 SMA mice at P11 that did not reach statistical significance. As whole tissue analysis was not sufficient to determine mRNA expression changes in the subset of spinal and DRG neurons that constitute the motor circuit, experiments were done to label and isolate these neurons specifically. The iliopsoas muscle participates in the righting reflex, which is severely affected in SMNΔ7 SMA mice (Mentis et al., 2011). The iliopsoas of SMNΔ7 SMA and control mice were injected at P2 with a fluorescently conjugated Cholera Toxin b (CTb) to label the motor neurons and proprioceptive neurons that connect to this muscle (FIG. 7C-D). At P6, immunohistochemistry against ChAT and parvalbumin demonstrated proper localization of CTb in the motor neurons and proprioceptive neurons associated with the iliopsoas muscle (FIG. 7E-F). The soma of these CTb-labeled neurons was isolated by laser capture microdissection (LCM) (FIG. 14E-F) and RT-qPCR analysis was done. There was a strong and specific enrichment of ChAT mRNA in LCM motor neurons and parvalbumin mRNA in proprioceptive neurons (FIG. 14G), confirming that the correct neuronal types were isolated. Stasimon U12 intron splicing and mRNA expression in motor circuit neurons isolated from control were compared with SMA-D7 mice. Remarkably, Stasimon U12 intron retention was strongly increased in both the motor neurons and proprioceptive neurons of SMNA7 SMA mice relative to controls (FIG. 7G), with the increase being most pronounced in proprioceptive neurons. Furthermore, Stasimon mRNA levels were also reduced in both types of motor circuit neurons from SMNA7 SMA mice compared to controls, again with a larger reduction observed in proprioceptive neurons. These results demonstrated that SMN deficiency disrupts Stasimon U12 splicing and mRNA expression in the constituent neurons of the sensory-motor circuit in a mouse model of SMA.

### Example 9

### Stasimon Mutants that Remove All Protein Function

Novel Stasimon deletion alleles remove all protein function. To begin to address the molecular function of stasimon, the P[EPgy2] transposon insertion EY04008 (Bellen et al., 2004; Lotti et al., 2012) in stas was mobilized in order to generate deletion alleles that would remove all protein function. The stas gene is located on the X chromosome (16B9). By conventional mobilization techniques and PCR screening, a novel stas deletion allele, stas^{Δ1} was recently identified, which is a 1.7 Kb deletion removing 33% of the coding region of the gene including the start codon and upstream sequence. Several additional smaller deletions were also identified. By mRNA in situ hybridization, stas^{Δ1} mutants appear to have no Stas mRNA expression. A portion of stas^{Δ1} hemizygous males can survive to adulthood and we are currently investigating if their frequency and adult lifespan is normal.

The mechano sensitive NompC channel is aberrantly localized in stas mutant proprioceptive neurons. Stasimon encodes an evolutionarily conserved protein containing six transmembrane domains and a region with homology to SNARE-associated Golgi proteins. Stas protein has been shown to interact with Drosophila Sec24 in large-scale protein interaction studies (Su et al., 2009). Sec24 mediates the selective export of membrane proteins from the endoplasmic reticulum. These results suggested the hypothesis that Stas may be required for membrane protein trafficking in neurons.

Though Stas is broadly expressed in many neurons, phenotypic rescue of smn mutants indicates that its critical role in motor circuit function is in proprioceptive neurons in Drosophila (Imlach et al., 2012; Lotti et al., 2012) and this activity appears to be conserved in mice. In Drosophila, both bd and type I md sensory neurons are essential components of a proprioceptive sensory feedback circuit necessary for coordinated contractile locomotion of Drosophila larvae. larvae (Hughes and Thomas, 2007) Sensory feedback does not seem to be necessary for Drosophila larval central pattern generator assembly or basic embryonic and larval movement (Crisp et al., 2008), however without sensory input, both rhythmic motor circuit activity and coordinated locomotion behavior are severely disrupted. Both bd and type I md subset of sensory neurons express the mechanosensitive NompC TRP channel which is essential for proprioception. Given that Stas may be required for protein trafficking in proprioceptive neurons and that correct NompC localization is also essential for proprioception, experiments were designed to see if NompC was correctly localized in stas^{Δ1} mutants.

Only bd and type I md proprioceptive sensory neurons were labeled with UAS-CD8GFP in stas^{Δ1} mutants and controls by using the selective driver NP2225-Gal4. NompC distribution in these animals was studied using a NompC specific antibody (Eijkelkamp et al., 2012). NompC is aberrantly increased in the soma of stas mutant proprioceptive neurons. Control and staΔ1 mutant proprioceptive neurons were labeled by GFP expressed using NP2225-Gal4 and with anti-NompC. In contrast to the diffuse labeling in control neurons, NompC was accumulated in the soma of stas mutant This result was consistent with trafficking of NompC being disrupted in bd and type I and sensory neurons sensory neurons in stas mutants, which may contribute to the proprioceptive defects.

Stas stas^{Δ1} full deletion mutants have normal responses to touch and nociception. To determine if stas mutants have general defects in sensory perception, we evaluated their responses to touch and nociceptive stimuli using established protocols (Branchereau et al., 2000; Wang et al., 2011). We found that the response of stas^{Δ1} mutant larvae to both sensory stimuli was similar to control animals (data not shown). This result indicates that stas mutants do not have a general defect in sensory perception. Interestingly, in addition to its role in proprioception, NompC has been shown to be necessary for response to touch in different class of sensory neurons [class III da neurons. (LaJeunesse et al., 2004). This may indicate a unique role for Stas in proprioceptive sensory neuron trafficking that is not required in touch-responsive sensory neurons.

### Example 10

### Efficient In Vivo Transduction of Motor Circuit Neurons with AAV9 Vectors

Self-complementary AAV9 (scAAV) vectors were used for the expression of GFP, human SMN or human STASIMON in SMNA7 SMA mice. The open reading frame of the human SMN1 cDNA or human Stasimon/Tmem41b cDNA was cloned into an AAV2-based plasmid containing AAV inverted terminal repeats and the CAG promoter containing the chicken β-actin promoter and an upstream CMV enhancer element. The recombinant plasmids were each packaged into AAV serotype-9 capsid by triple-plasmid co-transfection of mammalian cells and virions were purified by two consecutive cesium chloride gradients and concentrated. SMNΔ7 SMA pups received a single injection into one cerebral lateral ventricle at P1.

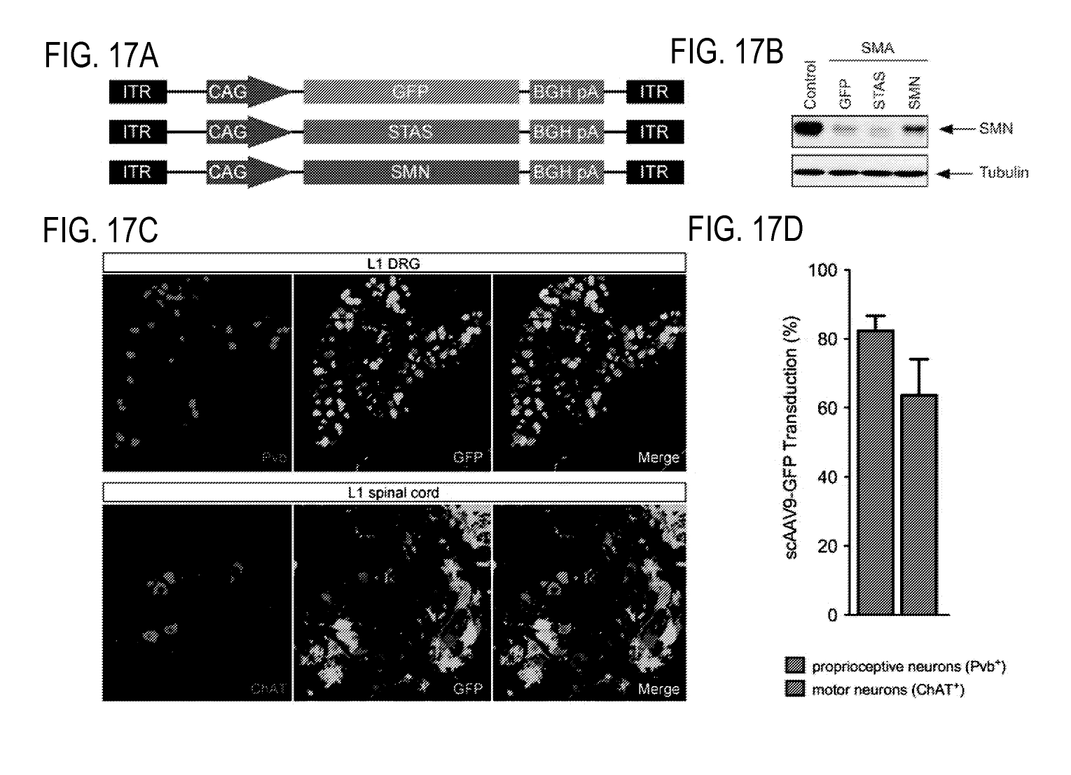

FIG. 17C shows immunohistochemistry and confocal microscopy images demonstrating robust GFP expression in motor circuit neurons, including parvalbumin (Pvb)⁺ proprioceptive neurons in the DRG and ChAT⁺ motor neurons in the spinal cord of scAAV9-GFP injected mice at P11. And FIG. 7D shows the percentage of GFP-expressing proprioceptive neurons and motor neurons following scAAV9-GFP injection, indicating that trasnduction of motor circuit neurons was highly efficient.

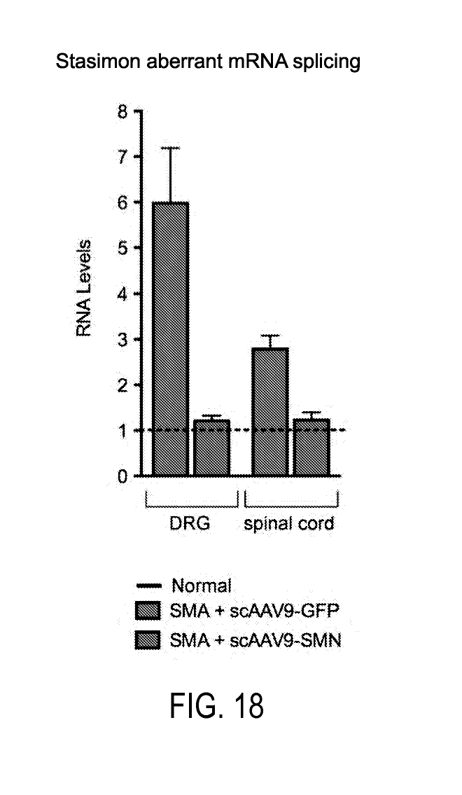

AAV-SMN gene therapy restored normal U12 splicing of Stasimon mRNA in SMNΔ7 SMA mice. RT-qPCR analysis of aberrant U12 splicing of Stasimon mRNA in the DRG and spinal cord of SMNΔ7 SMA mice injected with scAAV9-GFP and scAAV9-STAS relative to wild-type (Normal) mice. Mice were injected at P1 and tissue collected at P11. Note that SMN restores normal U12 splicing of Stasimon (FIG. 18), which correlates with phenotypic correction. FIG. 19 shows that AAV-STAS improved the SMA phenotype in SMNA7 SMA mice. Representative images of SMNA7 SMA mice injected with scAAV9-STAS and scAAV9-GFP at P14 are shown. Note that STAS-injected mice stand properly on the four limbs and are well-groomed, larger and healthier than GFP-injected controls. FIG. 20 shows that AAV-STAS improved weight gain in SMNΔ7 SMA mice. Note that STAS-injected SMNΔ7 SMA mice gain much more weight than GFP-injected controls. The effect of STAS on weight is comparable to if not better than that of SMN-injected mice until P12, after which it progressively declines. Finally, FIG. 21 shows that AAV-STAS increased lifespan in SMNA7 SMA mice. Kaplan-Meier survival plot of wild-type (Control) and SMNΔ7 SMA mice injected with the indicated scAAV9-vectors. Note that STAS injection leads to a 25% increase in the lifespan of SMAD7 mice (GFP median survival=14 days; STAS median survival=17.5 days; SMN median survival=40 days).

### Example 11

### SMN-Dependent Genes Rashomon that Contribute to Other Motor Circuit Functions Disrupted by SMN Depletion

From a genome-wide screen, 7 genes were found that (a) contained a U12 intron and (b) had reduced expression in smn mutants (Lotti et al., 2012). Ubiquitous RNAi inhibition of one of these genes, Stasimon, had similar NMJ defects to smn mutants, while inhibition of all of the others did not While restoration of Stasimon expression fully corrected defects in NMJ electrophysiology and partially corrected defects in muscle growth, defects in motor rhythm or locomotion were not corrected at all. Therefore, defective expression of one or more of these 6 other genes theoretically could contribute to aspects of the Drosophila smn mutant phenotype. To test this hypothesis, RNAi inhibition was used to screen (and mutants where available) each of these other genes for defects in muscle size, locomotion [using quantitative video trafficking] and motor rhythm [using electrophysiology] (Imlach et al., 2012).

CG33108/Rashomon is identified as a novel SMN-dependent gene required for normal locomotion. From this screen, it was discovered that ubiquitous RNAi inhibition of the uncharacterized gene CG33108 with Da-Gal4 reduced larval locomotion, while inhibition of each of the 5 other genes did not. CG33108 is predicted to encode two protein isoforms (267AA and 180AA) conserved with the predicted products of the human gene C19orf54 (29% amino acid identity, 45% conservation) [FIG. 10]. We identified a number of existing transposon insertions predicted to disrupt CG33108 including the PBac[WH] insertion f01565 and P[EPgy2] EY04458. We examined the locomotion of these mutants in trans-heterozygous combinations and confirmed that these mutants also reduced larval locomotion. As CG33108 expression is reduced by SMN depletion and both knockdown and mutants of CG33108 caused a locomotion phenotype similar to that of smn mutants, we renamed CG33108 Rashomon (reduced in smn)—abbreviated as rash—and investigated its function further.

Both Human C19orf54 and Drosophila Rashomon(also abbreviated as Rash) are conserved. The rash gene makes two predicted gene products and is disrupted by a number of existing transposon insertions. Rash mRNA is expressed in a bilateral subset of neurons in the embryonic nervous system. Importantly, expression of a Rash cDNA increased smn mutant locomotion speed while rash mutants or RNAi inhibition of Rash resulted in reduced larval locomotion.

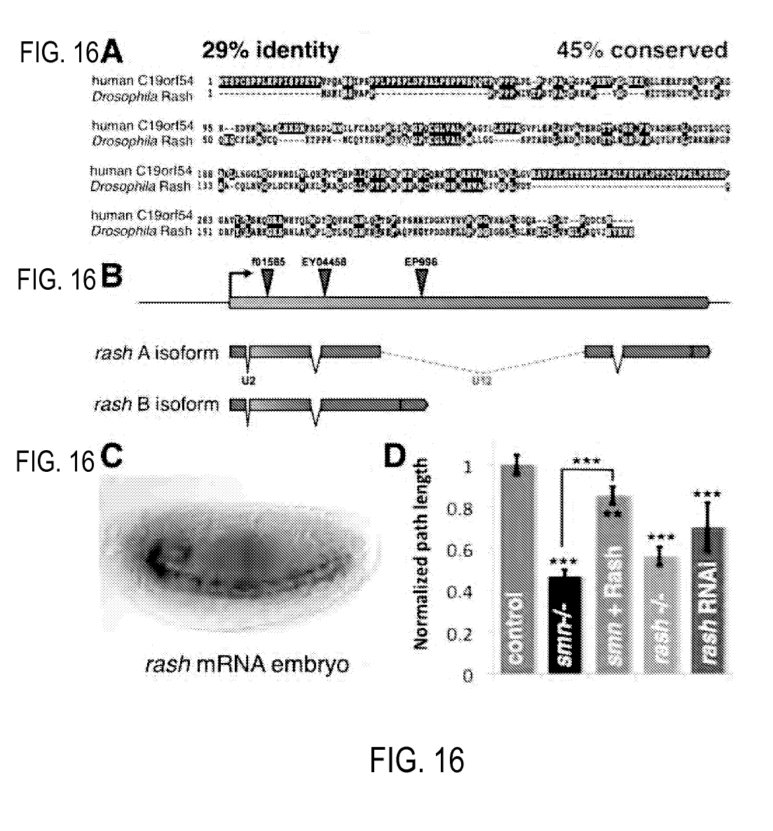

Rash is expressed in CNS neurons and partially rescued the locomotion of smn mutants. Rash mRNA in the Drosophila embryonic nervous system (studied by in situ hybridization) is strongly expressed in bilateral subsets of CNS neurons both in the brain lobes and ventral nerve cord. This expression pattern was consistent with a potential role in locomotion that was observed in SMN mutants. Restoration of Rash by expression of UAS-Rash in all neurons rescued smn mutants. By increasing the locomotion speed of smn mutants by 110% (P<0.001) however these animals were significantly slower than controls. It is noteworthy however that proprioception is likely still defective in these animals since Stas is reduced. Expression of Stas and Rash may further increase locomotion with additive or synergistic effects. FIG. 16.

mRash expression is disrupted in the spinal cord of SMA model mice. Drosophila, mouse and human rash homologs have both conserved amino acid sequence and the conserved presence of a U12 intron. Furthermore, in SMN-depleted NIH3T3 cells, mouse C19orf54/mRash expression is reduced by ˜50% [FIG. 11. Given the results in Drosophila, experiments were done to test if U12 splicing and mRNA expression of mRash was affected in the DRG and spinal cord of SMNΔ7 SMA mice at P1, P6 or P11, as well as if any defects were restored in SMNΔ7 SMA mice that have been rescued with scAAV9-SMN. The results show that analogous to mStas, aberrant U12-intron retention was detected in the spinal cords of SMA mice. In late-symptomatic P11 SMA mice reduced mRNA expression was also detected in the spinal cord. However, in contrast to mStas, both U12-intron retention and mRNA expression of mRash were not reduced in DRG neurons. This was consistent with central rather than peripheral expression of Rash in Drosophila neurons. mRash U12 intron retention was fully corrected in SMNΔ7 rescued by AAV delivered SMN. These results are consistent with Rash being a SMN-dependent gene required in the motor circuits of both Drosophila and mouse SMA models.

### REFERENCES

Alioto, T. S. (2007). U12DB: a database of orthologous U12-type spliceosomal introns. Nucleic Acids Res 35, D110-115.
Baumer, D., Lee, S., Nicholson, G., Davies, J. L., Parkinson, N. J., Murray, L. M., Gillingwater, T. H., Ansorge, O., Davies, K. E., and Talbot, K. (2009). Alternative splicing events are a late feature of pathology in a mouse model of spinal muscular atrophy. PLoS Genet 5, e1000773.
Beattie, C. E., Carrel, T. L., and McWhorter, M. L. (2007). Fishing for a mechanism: using zebrafish to understand spinal muscular atrophy. J Child Neurol 22, 995-1003.
Bellen, H. J., Levis, R. W., Liao, G., He, Y., Carlson, J. W., Tsang, G., Evans-Holm, M., Hiesinger, P. R., Schulze, K. L., Rubin, G. M., et al. (2004). The BDGP gene disruption project: single transposon insertions associated with 40% of Drosophila genes. Genetics 167, 761-781.
Boulisfane, N., Choleza, M., Rage, F., Neel, H., Soret, J., and Bordonne, R. (2011). Impaired minor tri-snRNP assembly generates differential splicing defects of U12-type introns in lymphoblasts derived from a type I SMA patient. Hum Mol Genet 20, 641-648.
Buhler, D., Raker, V., Luhrmann, R., and Fischer, U. (1999). Essential role for the tudor domain of SMN in spliceosomal U snRNP assembly: implications for spinal muscular atrophy. Hum Mol Genet 8, 2351-2357.
Burghes, A. H., and Beattie, C. E. (2009). Spinal muscular atrophy: why do low levels of survival motor neuron protein make motor neurons sick? Nat Rev Neurosci 10, 597-609.
Carrel, T. L., McWhorter, M. L., Workman, E., Zhang, H., Wolstencroft, E.C., Lorson, C., Bassell, G. J., Burghes, A. H., and Beattie, C. E. (2006). Survival motor neuron function in motor axons is independent of functions required for small nuclear ribonucleoprotein biogenesis. J Neurosci 26, 11014-11022.
Chan, Y. B., Miguel-Aliaga, I., Franks, C., Thomas, N., Trulzsch, B., Sattelle, D. B., Davies, K. E., and van den Heuvel, M. (2003). Neuromuscular defects in a Drosophila survival motor neuron gene mutant. Hum Mol Genet 12, 1367-1376.
Chang, H. C., Dimlich, D. N., Yokokura, T., Mukherjee, A., Kankel, M. W., Sen, A., Sridhar, V., Fulga, T. A., Hart, A. C., Van Vactor, D., et al. (2008). Modeling spinal muscular atrophy in Drosophila. PLoS One 3, e3209.

Cooper, T. A., Wan, L., and Dreyfuss, G. (2009). RNA and disease. Cell 136, 777-793.
Dalgin, G., Ward, A. B., Hao le, T., Beattie, C. E., Nechiporuk, A., and Prince, V. E. (2011). Zebrafish mnxl controls cell fate choice in the developing endocrine pancreas. Development 138, 4597-4608.
Dietzl, G., Chen, D., Schnorrer, F., Su, K. C., Barinova, Y., Fellner, M., Gasser, B., Kinsey, K., Oppel, S., Scheiblauer, S., et al. (2007). A genome-wide transgenic RNAi library for conditional gene inactivation in Drosophila. Nature 448, 151-156.
Gabanella, F., Butchbach, M. E., Saieva, L., Carissimi, C., Burghes, A. H., and Pellizzoni, L. (2007). Ribonucleoprotein assembly defects correlate with spinal muscular atrophy severity and preferentially affect a subset of spliceosomal snRNPs. PLoS One 2, e921.
Gabanella, F., Carissimi, C., Usiello, A., and Pellizzoni, L. (2005). The activity of the spinal muscular atrophy protein is regulated during development and cellular differentiation. Hum Mol Genet 14, 3629-3642.
Imlach, W., and McCabe, B. D. (2009). Electrophysiological methods for recording synaptic potentials from the NMJ of Drosophila larvae. J Vis Exp. 10, 1109
Imlach, L. W., Beck, E. S., Choi, B. J., Lotti, F., Pellizzoni, L., and McCabe, B. D. (2012). SMN is required for sensory-motor circuit function in Drosophila. Cell.
Jodelka, F. M., Ebert, A. D., Duelli, D. M., and Hastings, M. L. (2010). A feedback loop regulates splicing of the spinal muscular atrophy-modifying gene, SMN2. Hum Mol Genet 19, 4906-4917.
Kabashi, E., Bercier, V., Lissouba, A., Liao, M., Brustein, E., Rouleau, G. A., and Drapeau, P. (2011). FUS and TARDBP but not SOD1 interact in genetic models of amyotrophic lateral sclerosis. PLoS Genet 7, e1002214.
Le, T. T., Pham, L. T., Butchbach, M. E., Zhang, H. L., Monani, U. R., Coovert, D. D., Gavrilina, T. O., Xing, L., Bassell, G. J., and Burghes, A. H. (2005). SMNDelta7, the major product of the centromeric survival motor neuron (SMN2) gene, extends survival in mice with spinal muscular atrophy and associates with full-length SMN. Hum Mol Genet 14, 845-857.
Lefebvre, S., Burglen, L., Reboullet, S., Clermont, O., Burlet, P., Viollet, L., Benichou, B., Cruaud, C., Millasseau, P., Zeviani, M., et al. (1995). Identification and characterization of a spinal muscular atrophy-determining gene. Cell 80, 155-165.
Lein, E. S., Hawrylycz, M. J., Ao, N., Ayres, M., Bensinger, A., Bernard, A., Boe, A. F., Boguski, M. S., Brockway, K. S., Byrnes, E. J., et al. (2007). Genome-wide atlas of gene expression in the adult mouse brain. Nature 445, 168-176.
Lin, C. F., Mount, S. M., Jarmolowski, A., and Makalowski, W. (2010). Evolutionary dynamics of U12-type spliceosomal introns. BMC Evol Biol 10, 47.
McWhorter, M. L., Monani, U. R., Burghes, A. H., and Beattie, C. E. (2003). Knockdown of the survival motor neuron (Smn) protein in zebrafish causes defects in motor axon outgrowth and pathfinding. J Cell Biol 162, 919-931.
Meister, G., Buhler, D., Pillai, R., Lottspeich, F., and Fischer, U. (2001). A multiprotein complex mediates the ATP-dependent assembly of spliceosomal U snRNPs. Nat Cell Biol 3, 945-949.
Mentis, G. Z., Blivis, D., Liu, W., Drobac, E., Crowder, M. E., Kong, L., Alvarez, F. J., Sumner, C. J., and O'Donovan, M. J. (2011). Early functional impairment of sensory-motor connectivity in a mouse model of spinal muscular atrophy. Neuron 69, 453-467.
Neuenkirchen, N., Chari, A., and Fischer, U. (2008). Deciphering the assembly pathway of Sm-class U snRNPs. FEBS Lett 582, 1997-2003.
Otake, L. R., Scamborova, P., Hashimoto, C., and Steitz, J. A. (2002). The divergent U12-type spliceosome is required for pre-mRNA splicing and is essential for development in Drosophila. Mol Cell 9, 439-446.
Patel, A. A., McCarthy, M., and Steitz, J. A. (2002). The splicing of U12-type introns can be a rate-limiting step in gene expression. Embo J 21, 3804-3815.
Patel, A. A., and Steitz, J. A. (2003). Splicing double: insights from the second spliceosome. Nat Rev Mol Cell Biol 4, 960-970.
Pellizzoni, L. (2007). Chaperoning ribonucleoprotein biogenesis in health and disease. EMBO Rep 8, 340-345.
Pellizzoni, L., Charroux, B., and Dreyfuss, G. (1999). SMN mutants of spinal muscular atrophy patients are defective in binding to snRNP proteins. Proc Natl Acad Sci USA 96, 11167-11172.
Pellizzoni, L., Yong, J., and Dreyfuss, G. (2002). Essential role for the SMN complex in the specificity of snRNP assembly. Science 298, 1775-1779.
Praveen, K., Wen, Y., and Matera, A. G. (2012). A Drosophila Model of Spinal Muscular Atrophy Uncouples snRNP Biogenesis Functions of Survival Motor Neuron from Locomotion and Viability Defects. Cell reports 1, 624-631.
Ruggiu, M., McGovern, V. L., Lotti, F., Saieva, L., Li, D. K., Kariya, S., Monani, U. R., Burghes, A. H., and Pellizzoni, L. (2012). A role for SMN exon 7 splicing in the selective vulnerability of motor neurons in spinal muscular atrophy. Mol Cell Biol 32, 126-138.
Sun, Y., Grimmler, M., Schwarzer, V., Schoenen, F., Fischer, U., and Wirth, B. (2005). Molecular and functional analysis of intragenic SMN1 mutations in patients with spinal muscular atrophy. Hum Mutat 25, 64-71.
Wan, L., Battle, D. J., Yong, J., Gubitz, A. K., Kolb, S. J., Wang, J., and Dreyfuss, G. (2005). The survival of motor neurons protein determines the capacity for snRNP assembly: biochemical deficiency in spinal muscular atrophy. Mol Cell Biol 25, 5543-5551.
Winkler, C., Eggert, C., Gradl, D., Meister, G., Giegerich, M., Wedlich, D., Laggerbauer, B., and Fischer, U. (2005). Reduced U snRNP assembly causes motor axon degeneration in an animal model for spinal muscular atrophy. Genes & Dev 19, 2320-2330.
Workman, E., Saieva, L., Carrel, T. L., Crawford, T. O., Liu, D., Lutz, C., Beattie, C. E., Pellizzoni, L., and Burghes, A. H. (2009). A SMN missense mutation complements SMN2 restoring snRNPs and rescuing SMA mice. Hum Mol Genet 18, 2215-2229.
Zhang, Z., Lotti, F., Dittmar, K., Younis, I., Wan, L., Kasim, M., and Dreyfuss, G. (2008). SMN deficiency causes tissue-specific perturbations in the repertoire of snRNAs and widespread defects in splicing. Cell 133, 585-600.
Alioto, T. S. (2007). U12DB: a database of orthologous U12-type spliceosomal introns. Nucleic Acids Res 35, D110-115.
Baumer, D., Lee, S., Nicholson, G., Davies, J. L., Parkinson, N. J., Murray, L. M., Gillingwater, T. H., Ansorge, O., Davies, K. E., and Talbot, K. (2009). Alternative splicing events are a late feature of pathology in a mouse model of spinal muscular atrophy. PLoS Genet 5, e1000773.
Beattie, C. E., Carrel, T. L., and McWhorter, M. L. (2007). Fishing for a mechanism: using zebrafish to understand spinal muscular atrophy. J Child Neurol 22, 995-1003.
Bellen, H. J., Levis, R. W., Liao, G., He, Y., Carlson, J. W., Tsang, G., Evans-Holm, M., Hiesinger, P. R., Schulze, K. L., Rubin, G. M., et al. (2004). The BDGP gene disruption project: single transposon insertions associated with 40% of Drosophila genes. Genetics 167, 761-781.
Boulisfane, N., Choleza, M., Rage, F., Neel, H., Soret, J., and Bordonne, R. (2011). Impaired minor tri-snRNP assembly generates differential splicing defects of U12-type introns in lymphoblasts derived from a type I SMA patient. Hum Mol Genet 20, 641-648.
Buhler, D., Raker, V., Luhrmann, R., and Fischer, U. (1999). Essential role for the tudor domain of SMN in spliceosomal U snRNP assembly: implications for spinal muscular atrophy. Hum Mol Genet 8, 2351-2357.
Burghes, A. H., and Beattie, C. E. (2009). Spinal muscular atrophy: why do low levels of survival motor neuron protein make motor neurons sick? Nat Rev Neurosci 10, 597-609.
Carrel, T. L., McWhorter, M. L., Workman, E., Zhang, H., Wolstencroft, E. C., Lorson, C., Bassell, G. J., Burghes, A. H., and Beattie, C. E. (2006). Survival motor neuron function in motor axons is independent of functions required for small nuclear ribonucleoprotein biogenesis. J Neurosci 26, 11014-11022.
Chan, Y. B., Miguel-Aliaga, I., Franks, C., Thomas, N., Trulzsch, B., Sattelle, D. B., Davies, K. E., and van den Heuvel, M. (2003). Neuromuscular defects in a Drosophila survival motor neuron gene mutant. Hum Mol Genet 12, 1367-1376.
Chang, H. C., Dimlich, D. N., Yokokura, T., Mukherjee, A., Kankel, M. W., Sen, A., Sridhar, V., Fulga, T. A., Hart, A. C., Van Vactor, D., et al. (2008). Modeling spinal muscular atrophy in Drosophila. PLoS One 3, e3209.
Cooper, T. A., Wan, L., and Dreyfuss, G. (2009). RNA and disease. Cell 136, 777-793.
Dalgin, G., Ward, A. B., Hao le, T., Beattie, C. E., Nechiporuk, A., and Prince, V. E. (2011). Zebrafish mnx1 controls cell fate choice in the developing endocrine pancreas. Development 138, 4597-4608.
Dietzl, G., Chen, D., Schnorrer, F., Su, K. C., Barinova, Y., Fellner, M., Gasser, B., Kinsey, K., Oppel, S., Scheiblauer, S., et al. (2007). A genome-wide transgenic RNAi library for conditional gene inactivation in Drosophila. Nature 448, 151-156.
Gabanella, F., Butchbach, M. E., Saieva, L., Carissimi, C., Burghes, A. H., and Pellizzoni, L. (2007). Ribonucleoprotein assembly defects correlate with spinal muscular atrophy severity and preferentially affect a subset of spliceosomal snRNPs. PLoS One 2, e921.
Gabanella, F., Carissimi, C., Usiello, A., and Pellizzoni, L. (2005). The activity of the spinal muscular atrophy protein is regulated during development and cellular differentiation. Hum Mol Genet 14, 3629-3642.
Imlach, W., and McCabe, B. D. (2009). Electrophysiological methods for recording synaptic potentials from the NMJ of Drosophila larvae. J Vis Exp. 10, 1109
Imlach, L. W., Beck, E. S., Choi, B. J., Lotti, F., Pellizzoni, L., and McCabe, B. D. (2012). SMN is required for sensory-motor circuit function in Drosophila. Cell.
Jodelka, F. M., Ebert, A. D., Duelli, D. M., and Hastings, M. L. (2010). A feedback loop regulates splicing of the spinal muscular atrophy-modifying gene, SMN2. Hum Mol Genet 19, 4906-4917.
Kabashi, E., Bercier, V., Lissouba, A., Liao, M., Brustein, E., Rouleau, G. A., and Drapeau, P. (2011). FUS and TARDBP but not SOD1 interact in genetic models of amyotrophic lateral sclerosis. PLoS Genet 7, e1002214.
Le, T. T., Pham, L. T., Butchbach, M. E., Zhang, H. L., Monani, U. R., Coovert, D. D., Gavrilina, T. O., Xing, L., Bassell, G. J., and Burghes, A. H. (2005). SMNDelta7, the major product of the centromeric survival motor neuron (SMN2) gene, extends survival in mice with spinal muscular atrophy and associates with full-length SMN. Hum Mol Genet 14, 845-857.
Lefebvre, S., Burglen, L., Reboullet, S., Clermont, O., Burlet, P., Viollet, L., Benichou, B., Cruaud, C., Millasseau, P., Zeviani, M., et al. (1995). Identification and characterization of a spinal muscular atrophy-determining gene. Cell 80, 155-165.
Lein, E. S., Hawrylycz, M. J., Ao, N., Ayres, M., Bensinger, A., Bernard, A., Boe, A. F., Boguski, M. S., Brockway, K. S., Byrnes, E. J., et al. (2007). Genome-wide atlas of gene expression in the adult mouse brain. Nature 445, 168-176.
Lin, C. F., Mount, S. M., Jarmolowski, A., and Makalowski, W. (2010). Evolutionary dynamics of U12-type spliceosomal introns. BMC Evol Biol 10, 47.
McWhorter, M. L., Monani, U. R., Burghes, A. H., and Beattie, C. E. (2003). Knockdown of the survival motor neuron (Smn) protein in zebrafish causes defects in motor axon outgrowth and pathfinding. J Cell Biol 162, 919-931.
Meister, G., Buhler, D., Pillai, R., Lottspeich, F., and Fischer, U. (2001). A multiprotein complex mediates the ATP-dependent assembly of spliceosomal U snRNPs. Nat Cell Biol 3, 945-949.
Mentis, G. Z., Blivis, D., Liu, W., Drobac, E., Crowder, M. E., Kong, L., Alvarez, F. J., Sumner, C. J., and O'Donovan, M. J. (2011). Early functional impairment of sensory-motor connectivity in a mouse model of spinal muscular atrophy. Neuron 69, 453-467.
Neuenkirchen, N., Chari, A., and Fischer, U. (2008). Deciphering the assembly pathway of Sm-class U snRNPs. FEBS Lett 582, 1997-2003.
Otake, L. R., Scamborova, P., Hashimoto, C., and Steitz, J. A. (2002). The divergent U12-type spliceosome is required for pre-mRNA splicing and is essential for development in Drosophila. Mol Cell 9, 439-446.
Patel, A. A., McCarthy, M., and Steitz, J. A. (2002). The splicing of U12-type introns can be a rate-limiting step in gene expression. Embo J 21, 3804-3815.
Patel, A. A., and Steitz, J. A. (2003). Splicing double: insights from the second spliceosome. Nat Rev Mol Cell Biol 4, 960-970.
Pellizzoni, L. (2007). Chaperoning ribonucleoprotein biogenesis in health and disease. EMBO Rep 8, 340-345.
Pellizzoni, L., Charroux, B., and Dreyfuss, G. (1999). SMN mutants of spinal muscular atrophy patients are defective in binding to snRNP proteins. Proc Natl Acad Sci USA 96, 11167-11172.
Pellizzoni, L., Yong, J., and Dreyfuss, G. (2002). Essential role for the SMN complex in the specificity of snRNP assembly. Science 298, 1775-1779.
Praveen, K., Wen, Y., and Matera, A. G. (2012). A Drosophila Model of Spinal Muscular Atrophy Uncouples snRNP Biogenesis Functions of Survival Motor Neuron from Locomotion and Viability Defects. Cell reports 1, 624-631.
Ruggiu, M., McGovern, V. L., Lotti, F., Saieva, L., Li, D. K., Kariya, S., Monani, U. R., Burghes, A. H., and Pellizzoni, L. (2012). A role for SMN exon 7 splicing in the selective vulnerability of motor neurons in spinal muscular atrophy. Mol Cell Biol 32, 126-138.
Sun, Y., Grimmler, M., Schwarzer, V., Schoenen, F., Fischer, U., and Wirth, B. (2005). Molecular and functional analysis of intragenic SMN1 mutations in patients with spinal muscular atrophy. Hum Mutat 25, 64-71.
Wan, L., Battle, D. J., Yong, J., Gubitz, A. K., Kolb, S. J., Wang, J., and Dreyfuss, G. (2005). The survival of motor neurons protein determines the capacity for snRNP assembly: biochemical deficiency in spinal muscular atrophy. Mol Cell Biol 25, 5543-5551.
Winkler, C., Eggert, C., Gradl, D., Meister, G., Giegerich, M., Wedlich, D., Laggerbauer, B., and Fischer, U. (2005). Reduced U snRNP assembly causes motor axon degeneration in an animal model for spinal muscular atrophy. Genes & Dev 19, 2320-2330.
Workman, E., Saieva, L., Carrel, T. L., Crawford, T. O., Liu, D., Lutz, C., Beattie, C. E., Pellizzoni, L., and Burghes, A. H. (2009). A SMN missense mutation complements SMN2 restoring snRNPs and rescuing SMA mice. Hum Mol Genet 18, 2215-2229.
Zhang, Z., Lotti, F., Dittmar, K., Younis, I., Wan, L., Kasim, M., and Dreyfuss, G. (2008). SMN deficiency causes tissue-specific perturbations in the repertoire of snRNAs and widespread defects in splicing. Cell 133, 585-600.
Aberle, H., Haghighi, A. P., Fetter, R. D., McCabe, B. D., Magalhaes, T. R., and Goodman, C. S. (2002). wishful thinking encodes a BMP type II receptor that regulates synaptic growth in Drosophila. Neuron 33, 545-558.
Arber, S., Ladle, D. R., Lin, J. H., Frank, E., and Jessell, T. M. (2000). ETS gene Er81 controls the formation of functional connections between group Ia sensory afferents and motor neurons. Cell 101, 485-498.
Bellen, H. J., Levis, R. W., Liao, G., He, Y., Carlson, J. W., Tsang, G., Evans-Holm, M., Hiesinger, P. R., Schulze, K. L., Rubin, G. M., et al. (2004). The BDGP gene disruption project: single transposon insertions associated with 40% of Drosophila genes. Genetics 167, 761-781.
Bushey, D., Tononi, G., and Cirelli, C. (2009). The Drosophila fragile X mental retardation gene regulates sleep need. J Neurosci 29, 1948-1961.
Carissimi, C., Saieva, L., Gabanella, F., and Pellizzoni, L. (2006). Gemin8 is required for the architecture and function of the survival motor neuron complex. J Biol Chem 281, 37009-37016.
Carrel, T. L., McWhorter, M. L., Workman, E., Zhang, H., Wolstencroft, E. C., Lorson, C., Bassell, G. J., Burghes, A. H., and Beattie, C. E. (2006). Survival motor neuron function in motor axons is independent of functions required for small nuclear ribonucleoprotein biogenesis. J Neurosci 26, 11014-11022.
Chan, Y. B., Miguel-Aliaga, I., Franks, C., Thomas, N., Trulzsch, B., Sattelle, D. B., Davies, K. E., and van den Heuvel, M. (2003). Neuromuscular defects in a Drosophila survival motor neuron gene mutant. Hum Mol Genet 12, 1367-1376.
Chang, H. C., Dimlich, D. N., Yokokura, T., Mukherjee, A., Kankel, M. W., Sen, A., Sridhar, V., Fulga, T. A., Hart, A. C., Van Vactor, D., et al. (2008). Modeling spinal muscular atrophy in Drosophila. PLoS One 3, e3209.
Dalgin, G., Ward, A. B., Hao le, T., Beattie, C. E., Nechiporuk, A., and Prince, V. E. (2011). Zebrafish mnx1 controls cell fate choice in the developing endocrine pancreas. Development 138, 4597-4608.

Dietzl, G., Chen, D., Schnorrer, F., Su, K. C., Barinova, Y., Fellner, M., Gasser, B., Kinsey, K., Oppel, S., Scheiblauer, S., et al. (2007). A genome-wide transgenic RNAi library for conditional gene inactivation in Drosophila. Nature 448, 151-156.
Dull, T., Zufferey, R., Kelly, M., Mandel, R. J., Nguyen, M., Trono, D., and Naldini, L. (1998). A third-generation lentivirus vector with a conditional packaging system. J Virol 72, 8463-8471.
Gabanella, F., Butchbach, M. E., Saieva, L., Carissimi, C., Burghes, A. H., and Pellizzoni, L. (2007). Ribonucleoprotein assembly defects correlate with spinal muscular atrophy severity and preferentially affect a subset of spliceosomal snRNPs. PLoS One 2, e921.
Gabanella, F., Carissimi, C., Usiello, A., and Pellizzoni, L. (2005). The activity of the spinal muscular atrophy protein is regulated during development and cellular differentiation. Hum Molecular Genet 14, 3629-3642.
Groth, A. C., Fish, M., Nusse, R., and Calos, M. P. (2004). Construction of transgenic Drosophila by using the site-specific integrase from phage phiC31. Genetics 166, 1775-1782.
Imlach, W., and McCabe, B. D. (2009). Electrophysiological methods for recording synaptic potentials from the NMJ of Drosophila larvae. J Vis Exp. 10, 1109.
Imlach, L. W., Beck, E. S., Choi, B. J., Lotti, F., Pellizzoni, L., and McCabe, B. D. (2012). SMN is required for sensory-motor circuit function in Drosophila. Cell.
Kabashi, E., Bercier, V., Lissouba, A., Liao, M., Brustein, E., Rouleau, G. A., and Drapeau, P. (2011). FUS and TARDBP but not SOD1 interact in genetic models of amyotrophic lateral sclerosis. PLoS Genet 7, e1002214.
Kosman, D., Mizutani, C. M., Lemons, D., Cox, W. G., McGinnis, W., and Bier, E. (2004). Multiplex detection of RNA expression in Drosophila embryos. Science 305, 846.
Langenau, D. M., Jette, C., Berghmans, S., Palomero, T., Kanki, J. P., Kutok, J. L., and Look, A. T. (2005). Suppression of apoptosis by bcl-2 overexpression in lymphoid cells of transgenic zebrafish. Blood 105, 3278-3285.
Le, T. T., Pham, L. T., Butchbach, M. E., Zhang, H. L., Monani, U. R., Coovert, D. D., Gavrilina, T. O., Xing, L., Bassell, G. J., and Burghes, A. H. (2005). SMNDelta7, the major product of the centromeric survival motor neuron (SMN2) gene, extends survival in mice with spinal muscular atrophy and associates with full-length SMN. Hum Mol Genet 14, 845-857.
Lein, E. S., Hawrylycz, M. J., Ao, N., Ayres, M., Bensinger, A., Bernard, A., Boe, A. F., Boguski, M. S., Brockway, K. S., Byrnes, E. J., et al. (2007). Genome-wide atlas of gene expression in the adult mouse brain. Nature 445, 168-176.
Lin, D. M., and Goodman, C. S. (1994). Ectopic and increased expression of Fasciclin II alters motoneuron growth cone guidance. Neuron 13, 507-523.
Mahr, A., and Aberle, H. (2006). The expression pattern of the Drosophila vesicular glutamate transporter: a marker protein for motoneurons and glutamatergic centers in the brain. Gene Expr Patterns 6, 299-309.
McWhorter, M. L., Monani, U. R., Burghes, A. H., and Beattie, C. E. (2003). Knockdown of the survival motor neuron (Smn) protein in zebrafish causes defects in motor axon outgrowth and pathfinding. J Cell Biol 162, 919-931.
Mentis, G. Z., Blivis, D., Liu, W., Drobac, E., Crowder, M. E., Kong, L., Alvarez, F. J., Sumner, C. J., and O'Donovan, M. J. (2011). Early functional impairment of sensory-motor connectivity in a mouse model of spinal muscular atrophy. Neuron 69, 453-467.
Oprea, G. E., Krober, S., McWhorter, M. L., Rossoll, W., Muller, S., Krawczak, M., Bassell, G. J., Beattie, C. E., and Wirth, B. (2008). Plastin 3 is a protective modifier of autosomal recessive spinal muscular atrophy. Science 320, 524-527.

Otake, L. R., Scamborova, P., Hashimoto, C., and Steitz, J. A. (2002). The divergent U12-type spliceosome is required for pre-mRNA splicing and is essential for development in Drosophila. Mol Cell 9, 439-446.
Pellizzoni, L., Yong, J., and Dreyfuss, G. (2002). Essential role for the SMN complex in the specificity of snRNP assembly. Science 298, 1775-1779.
Perrin, L., Bloyer, S., Ferraz, C., Agrawal, N., Sinha, P., and Dura, J. M. (2003). The leucine zipper motif of the Drosophila AF10 homologue can inhibit PRE-mediated repression: implications for leukemogenic activity of human MLL-AF10 fusions. Mol Cell Biol 23, 119-130.
Rubin, G. M., Yandell, M. D., Wortman, J. R., Gabor Miklos, G. L., Nelson, C. R., Hariharan, I. K., Fortini, M. E., Li, P. W., Apweiler, R., Fleischmann, W., et al. (2000). Comparative genomics of the eukaryotes. Science 287, 2204-2215.
Salvaterra, P. M., and Kitamoto, T. (2001). Drosophila cholinergic neurons and processes visualized with Gal4/UAS-GFP. Brain Res Gene Expr Patterns 1, 73-82.
Wang, J. W., Beck, E. S., and McCabe, B. D. (2012). A modular toolset for recombination transgenesis and neurogenetic analysis of Drosophila. PloS One 7, e42102.
Workman, E., Saieva, L., Carrel, T. L., Crawford, T. O., Liu, D., Lutz, C., Beattie, C. E., Pellizzoni, L., and Burghes, A. H. (2009). A SMN missense mutation complements SMN2 restoring snRNPs and rescuing SMA mice. Hum Mol Genet 18, 2215-2229.
Zufferey, R., Dull, T., Mandel, R. J., Bukovsky, A., Quiroz, D., Naldini, L., and Trono, D. (1998). Self-inactivating lentivirus vector for safe and efficient in vivo gene delivery. J Virol 72, 9873-9880.
Bellen, H. J., Levis, R. W., Liao, G., He, Y., Carlson, J. W., Tsang, G., Evans-Holm, M., Hiesinger, P. R., Schulze, K. L., Rubin, G. M., et al. (2004). The BDGP gene disruption project: single transposon insertions associated with 40% of Drosophila genes. Genetics 167, 761-781.
Branchereau, P., Morin, D., Bonnot, A., Ballion, B., Chapron, J., and Viala, D. (2000). Development of lumbar rhythmic networks: from embryonic to neonate locomotor-like patterns in the mouse. Brain research bulletin 53, 711-718.
Cheng, L. E., Song, W., Looger, L. L., Jan, L. Y., and Jan, Y. N. (2010). The role of the TRP channel NompC in Drosophila larval and adult locomotion. Neuron 67, 373-380.
Crisp, S., Evers, J. F., Fiala, A., and Bate, M. (2008). The development of motor coordination in Drosophila embryos. Development 135, 3707-3717.
Eijkelkamp, N., Linley, J. E., Baker, M. D., Minett, M. S., Cregg, R., Werdehausen, R., Rugiero, F., and Wood, J. N. (2012). Neurological perspectives on voltage-gated sodium channels. Brain: a journal of neurology 135, 2585-2612.
Fox, L. E., Soll, D. R., and Wu, C. F. (2006). Coordination and modulation of locomotion pattern generators in Drosophila larvae: effects of altered biogenic amine levels by the tyramine beta hydroxlyase mutation. J Neurosci 26, 1486-1498.
Hughes, C. L., and Thomas, J. B. (2007). A sensory feedback circuit coordinates muscle activity in Drosophila. Mol Cell Neurosci 35, 383-396.
Imlach, W. L., Beck, E. S., Choi, B. J., Lotti, F., Pellizzoni, L., and McCabe, B. D. (2012). SMN Is Required for Sensory-Motor Circuit Function in Drosophila. Cell 151, 427-439.
LaJeunesse, D. R., Buckner, S. M., Lake, J., Na, C., Pirt, A., and Fromson, K. (2004). Three new Drosophila markers of intracellular membranes. BioTechniques 36, 784-788, 790.
Lotti, F., Imlach, W. L., Saieva, L., Beck, E.S., Hao le, T., Li, D. K., Jiao, W., Mentis, G. Z., Beattie, C. E., McCabe, B. D., et al. (2012). An SMN-Dependent U12 Splicing Event Essential for Motor Circuit Function. Cell 151, 440-454.
Remy, C., Remy, S., Beck, H., Swandulla, D., and Hans, M. (2004). Modulation of voltage-dependent sodium channels by the delta-agonist SNC80 in acutely isolated rat hippocampal neurons. Neuropharmacology 47, 1102-1112.
Song, W., Onishi, M., Jan, L. Y., and Jan, Y. N. (2007). Peripheral multidendritic sensory neurons are necessary for rhythmic locomotion behavior in Drosophila larvae. Proc Natl Acad Sci USA 104, 5199-5204.
Su, X., Castle, N. A., Antonio, B., Roeloffs, R., Thomas, J. B., Krafte, D. S., and Chapman, M. L. (2009). The effect of kappa-opioid receptor agonists on tetrodotoxin-resistant sodium channels in primary sensory neurons. Anesthesia and analgesia 109, 632-640.
Wang, J. W., Brent, J. R., Tomlinson, A., Shneider, N. A., and McCabe, B. D. (2011). The ALS-associated proteins FUS and TDP-43 function together to affect Drosophila locomotion and life span. The Journal of clinical investigation 121, 4118-4126.

What is claimed is:

1. A pharmaceutical composition comprising a pharmaceutically acceptable vehicle and a therapeutically effective amount of one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, Nucleolar protein 1, and any combination thereof, and any biologically active fragment or variant thereof.

2. A method for treating a motor neuron disease, comprising
a) identifying a subject having a symptom of the disease, and
b) administering to the subject the pharmaceutical composition of claim 1.

3. The pharmaceutical composition of claim 1 which is formulated for uptake by the brain, spinal cord or motor circuit neurons.

4. The method of claim 2 wherein the protein is Stasimon, Rashomon or a combination thereof, or a biologically active fragment or variant thereof.

5. The method of claim 2 where the therapeutically effective amount of the protein is an amount that increases muscle mass in the subject.

6. The method of claim 2 where the therapeutically effective amount of the protein is an amount that increases transmission from a neuromuscular junction.

7. A method for treating a motor neuron disease, comprising
a) identifying a subject having a symptom of the disease, and
b) administering to the subject a gene delivery vehicle comprising one or more genes selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, Nucleolar protein 1 and a biologically active fragment or variant thereof.

8. The method of claim 2 wherein the subject is mammalian.

9. The method of claim 7 wherein the subject is mammalian.

10. The method of claim 2 wherein the subject is human.

11. The method of claim 7 wherein the subject is human.

12. The method of claim 2 wherein the motor neuron disease is a member selected from the group comprising Amyotrophic lateral sclerosis (ALS), Progressive bulbar pals, Pseudobulbar palsy, Primary lateral sclerosis (PLS), Progressive muscular atrophy and spinal muscular atrophy (SMA).

13. The method of claim 7 wherein the motor neuron disease is a member selected from the group consisting of Amyotrophic lateral sclerosis (ALS), Progressive bulbar palsy, Pseudobulbar palsy, Primary lateral sclerosis (PLS), Progressive muscular atrophy and spinal muscular atrophy (SMA).

14. The method of claim 2 wherein the treatment is an SMN restoration therapy comprising administering a gene encoding SMN to the subject.

15. The method of claim 7 wherein the treatment is an SMN restoration therapy comprising administering a gene encoding SMN to the subject.

16. The method of claim 7 wherein the gene delivery vehicle is a viral vector comprising the one or more genes as cDNAs.

17. The method of claim 7 wherein the viral vector is an AAV comprising either a single stranded or a self-complementary genome comprising the one or more genes as cDNAs.

18. The method of claim 7 wherein the gene delivery vehicle is administered systemically.

19. The method of claim 7 wherein the gene delivery vehicle is delivered to brain.

20. The method of claim 7 wherein the gene delivery vehicle is delivered to spinal cord.

21. The method of claim 7, wherein the gene delivery vehicle is delivered to a motor circuit neuron.

22. A method for diagnosing a motor neuron disease comprising
a) identifying a subject with a symptom of a motor neuron disease,
b) obtaining a biological sample from the subject and a control biological sample from a normal subject known not to have a motor neuron disease,
c) determining in the subject sample and the control samples a level of an mRNA comprising an unspliced, abnormally spliced, or aberrantly spliced U12 intron which is transcribed from a U12 intron-containing gene selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or a level of correctly spliced mRNA encoding the one or more proteins,
d) if the level of protein or correctly spliced mRNA is significantly lower in the subject sample than in the control sample, then determining that the subject has the motor neuron disease, and
e) treating the subject the motor neuron disease.

23. A method for diagnosing a motor neuron disease comprising
a) identifying a subject with a symptom of a motor neuron disease,
b) obtaining a biological sample from the subject and a control biological sample from a normal subject known not to have a motor neuron disease,
c) determining in the subject sample and the control samples a level of one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or a level of correctly spliced mRNA encoding the one or more proteins,
d) if the level of protein or correctly spliced mRNA is significantly lower in the subject sample than in the control sample, then determining that the subject has the motor neuron disease, and
e) treating the subject the motor neuron disease.

24. A method for monitoring a response of a subject to treatment for a motor neuron disease, comprising
c) obtaining a pretreatment biological sample and a post treatment biological sample from the subject,
d) determining in the pretreatment and post treatment samples a level of one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or a level of correctly spliced mRNA encoding the one or more proteins,
e) and if the level of the protein or mRNA s significantly higher in the post treatment sample than in the pretreatment sample, then determining that the subject is responding to treatment for the motor neuron disease, and
f) continuing the treatment for the motor neuron disease.

25. A method for monitoring a response of a subject to treatment for a motor neuron disease, comprising
a) obtaining a pretreatment biological sample and a post treatment biological sample from the subject,
b) determining in the pretreatment and post treatment samples a level of an mRNA comprising an unspliced, abnormally spliced or aberrantly spliced U12 intron which mRNA is transcribed from a U12 intron-containing gene selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or a level of correctly spliced mRNA encoding the one or more proteins,
c) and if the level of the protein or mRNA s significantly higher in the post treatment sample than in the pretreatment sample, then determining that the subject is responding to treatment for the motor neuron disease, and
d) continuing the treatment for the motor neuron disease.

26. The method of claim 22 wherein the biological sample is a member selected from the group comprising fibroblasts, blood, serum, muscle and cerebrospinal fluid.

27. The method of claim 23 wherein the biological sample is a member selected from the group comprising fibroblasts, blood, serum, muscle and cerebrospinal fluid.

28. The method of claim 22 wherein the treatment of the subject in step e) includes administering to the subject a therapeutically effective amount of the protein determined in step d) to be present in the subject biological sample at a level that is significantly lower than in the control level of the protein.

29. The method of claim 23 wherein the treatment of the subject in step e) includes administering to the subject a therapeutically effective amount of the protein determined in step d) to be present in the subject biological sample at a level that is significantly lower than in the control level of the protein.

30. An AAV comprising a cDNA of a gene encoding one or more proteins selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1.

31. A microarray comprising two or more oligonucleotides bound to a support that are complementary to and selectively hybridize to one or more genes selected from the group consisting of Transmembrane protein 41B (Stasimon), Chromosome 19 open reading frame 54 (Rashomon), Tetraspanin 31, Poly (ADP-ribose) polymerase family member 1, Histidyl-tRNA synthetase-like, Chloride channel 7, and Nucleolar protein 1, or to one or more correctly spliced mRNAs encoded by the respective genes.
